(19) United States
(12) Patent Application Publication
(10) Pub. No.: US 2012/0100143 A1
(43) Pub. Date: Apr. 26, 2012

(54) IMMUNOGENIC PROTEINS FROM GENOME-DERIVED OUTER MEMBRANE OF LEPTOSPIRA AND COMPOSITIONS AND METHODS BASED THEREON

(76) Inventor: Yung-Fu Chang, Ithaca, NY (US)

(21) Appl. No.: 12/679,643

(22) PCT Filed: Sep. 22, 2008
(86) PCT No.: PCT/US08/77182

§ 371 Date: Jun. 23, 2010

Related U.S. Application Data
(60) Provisional application No. 60/974,818, filed on Sep. 24, 2007.
(60) Provisional application No. 60/976,088, filed on Sep. 28, 2007.

(51) Int. Cl. A61K 39/40; C07K 16/12; A61P 31/04; A61P 37/04; A61P 15/00; C12N 15/63; C12P 21/02; G01N 33/569; A61K 39/02; A61P 1/16

(52) U.S. Cl. 424/139.1; 424/190.1; 530/387.9; 435/320.1; 424/234.1; 435/69.3; 435/7.32

(57) ABSTRACT

Leptospira outer membrane proteins (OMPs) LP1454, LP1118, LP1939, MCEII, CADF-like1, CADF-like2, CADF-like3, Lp0022, Lp1499, Lp4337, Lp328 or L21 are provided. The OMPS can be used as tools for developing effective vaccines or diagnostic methods for leptospirosis. Expression vectors for the OMP genes are further provided. The antigenic properties of the Leptospira OMPs can be used to create, manufacture or improve vaccines. Vaccines, including but not limited to DNA vaccines, recombinant vaccines, and T-cell epitope vaccines based on the foregoing OMPs are also provided. Methods for producing such vaccines are also provided. Also provided are methods for using Leptospira OMP genes, proteins and antibodies for therapeutic treatment and serological diagnosis techniques.

### CROSS-REFERENCE TO RELATED APPLICATIONS

This application claims priority to and the benefit of co-pending U.S. provisional patent application Ser. No. 60/974,818 (filed Sep. 24, 2007) and Ser. No. 60/976,088 (filed Sep. 28, 2007), both of which are incorporated herein by reference in their entireties.

### STATEMENT REGARDING FEDERALLY SPONSORED RESEARCH OR DEVELOPMENT

Not applicable

### REFERENCE TO APPENDIX

Not applicable

### 1. TECHNICAL FIELD

The present invention relates to Leptospira outer membrane proteins (OMPs) and antibodies directed against them. The invention also relates to methods and compositions for the treatment of diseases or disorders caused by a blood-borne immunogenic Leptospira pathogen, comprising administering antibodies that bind to OMPs. The invention further relates to methods for using Leptospira OMPs as vaccines or diagnostic antigens.

### 2. BACKGROUND

Leptospira species are a gram negative spirochete, the causative agent of leptospirosis, a zoonotic disease of mammals characterized by renal and liver failure, abortion, uveitis, reproductive failure, myocarditis, mastitis and pulmonary hemorrhage (Kishimoto, et al. 2004. Leptospirosis misdiagnosed as pulmonary-renal syndrome. Am. J. Med. Sci. 328:116-120; Marotto, et al. 1999. Acute lung injury in leptospirosis: clinical and laboratory features, outcome, and factors associated with mortality. Clin. Infect. Dis, 29:1561-1563; Seijo, et al. 2002. Lethal leptospiral pulmonary hemorrhage: an emerging disease in Buenos Aires, Argentina. Emerg. Infect. Dis. 8:1004-1005; Trevejo, et al. 1998. Epidemic leptospirosis associated with pulmonary hemorrhage-Nicaragua, 1995. J. Infect. Dis. 178:1457-1463; Vinetz, J. M. 2001. Leptospirosis. Curr. Opin. Infect. Dis. 14:527-538).

Although there are 21 genomospecies of Leptospira, the most common cause of leptospirosis among cattle, especially in dairy cows in the United States, is L. borgpetersenii serovar Hardjo, and L. interrogans serovar Pomona (Talpada, M. D., N. Garvey, R. Sprowls, A. K. Eugster, and J. M. Vinetz. 2003. Prevalence of leptospiral infection in Texas cattle: implications for transmission to humans. Vector Borne Zoonotic Dis. 3:141-147). Cattle act as the maintenance host for this infection and in chronically infected cattle, leptospires persist in the kidneys and genital tract, leading to abortion (Ellis, W. A., and S. W. Michna. 1976. Bovine leptospirosis: demonstration of leptospires of the Hebdomadis serogroup in aborted fetuses and a premature calf. Vet. Rec. 99:430-432; Ellis. W. A., J. J. O'Brien, D. G. Bryson, and D. P. Mackie. 1985. Bovine leptospirosis: some clinical features of serovar Hardjo infection. Vet. Rec. 117:101-104) and renal failure (Masri, S. A., P. T. Nguyen, S. P. Gale, C. J. Howard, and S. C. Jung. 1997. A polymerase chain reaction assay for the detection of Leptospira spp. in bovine semen. Can. J. Vet. Res. 61:15-20). Leptospira species also infects pigs, horses, dogs and humans (Palaniappan, R. U. M.; Ramanujam, S.; Chang, Y-F. (2007) Leptospirosis: pathogenesis, immunity, and diagnosis. Curr. Opin. Infect. Dis. 20(3): 284-292). Infected animals may also survive asymptomatically by excreting the spirochetes intermittently in the urine, eventually affecting the other healthy animals.

Diagnosis of leptospirosis depends on a standard serological test, the microscopic agglutination test (MAT). However, MAT is time consuming, laborious and lack sensitivity to detect the infection at an early stage. Serological tests for diagnosis such as dipstick assay, indirect hemagglutination assay and enzyme linked immunosorbent assay (ELISA) are alternative methods for rapid diagnosis, but only few assays have been systematically evaluated based on the MAT titers (Palaniappan, et al. 2004. Expression of leptospiral immunoglobulin-like protein by Leptospira interrogans and evaluation of its diagnostic potential in a kinetic ELISA. J. Med. Microbiol. 53:975-984). This information is crucial for many diagnostic laboratories to choose the suitable assay for diagnosis of leptospirosis.

Commercially available monovalent or pentavalent vaccines provide only short-term immunity and are ineffective against increasing serovars of Leptospira. Recently, a new vaccine containing whole cell proteins of L. borgpetersenii serovar Hardjo has been found to protect even the transmission of infection from mother to fetus in cattle (Ribeiro, M. A., C. C. Souza, and S. H. Almeida. 1995. Dot-ELISA for human leptospirosis employing immunodominant antigen J. Trop. Med. Hyg. 98:452-456; Smith, C. R., P. J. Ketterer, M. R. McCGowan, and B. G. Corney 1994. A review of laboratory techniques and their use in the diagnosis of Leptospira interrogans serovar Hardjo infection in cattle. Aust. Vet. J. 71:290-294). Notably, these vaccines increase the antibody titer against leptospiral antigens in animals, and this increase cannot be differentiated by the standard microscopic agglutination test (MAT). Furthermore, if these vaccinated animals are also subjected to infection from other serovars of Leptospira, distinguishing vaccination from infection using MAT is impossible.

There is therefore a need in the art for a vaccine for leptospirosis. There is also a need for distinguishing leptospirosis vaccination from infection.

Citation or identification of any reference in Section 2, or in any other section of this application, shall not be considered an admission that such reference is available as prior art to the present invention.

### 3. SUMMARY OF THE INVENTION

Leptospira outer membrane proteins (OMPs) rLP1454 (SEQ ID NOS: 1-2), rLP1118 (SEQ ID NOS: 3-4), rLP1939 (SEQ ID NOS: 5-6), rMCEII (Lp0607; SEQ ID NOS: 7-8), CADF-like1 (SEQ ID NOS: 52-53; FIG. 6A), CADF-like2 (SEQ ID NOS: 54-55; FIG. 68), CADF-like3 (SEQ ID NOS: 56-57; FIG. 6B), Lp0022 (SEQ ID NOS: 58-59; FIG. 6C), Lp1499 (SEQ ID NOS: 60-61; FIG. 6D), Lp4337 (SEQ ID NOS: 62-63; FIG. 6E), Lp328 (SEQ ID NOS: 64-65; FIG. 6E) and L21 (SEQ ID NOS: 66-67; FIG. 6E) are provided that can serve as tools for developing effective vaccines or diagnostic methods for leptospirosis. In one embodiment, protein fragments of at least 10%, 20%, 30%, 40%, 50%, 60% 70%, 80%, 90% or 95% of the length of the amino acid sequence of any of the above are provided.

The antigenic properties of the Leptospira LP1454, LP1118, LP1939, MCEII, CADF-like1, CADF-like2, CADF-like3, Lp0022, Lp1499, Lp4337, Lp328 or L21 OMPs can be used to create, manufacture or improve vaccines. Vaccines, including but not limited to DNA vaccines, recombinant vaccines, and T-cell epitope vaccines based on the foregoing OMPs are further provided. Methods for producing such vaccines are also provided.

In one embodiment, the vaccine is based on a Leptospira outer membrane protein (OMP) selected from the group consisting of rLP1454 (SEQ ID NOS: 1-2), rLP1118 (SEQ ID NOS: 3-4), rLP1939 (SEQ ID NOS: 5-6), rMCEII (Lp0607; SEQ ID NOS: 7-8), CADF-like1 (SEQ ID NOS: 52-53; FIG. 6A), CADF-like2 (SEQ ID NOS: 54-55; FIG. 6B), CADF-like3 (SEQ ID NOS: 56-57; FIG. 6B), Lp0022 (SEQ ID NOS: 58-59; FIG. 6C), Lp1499 (SEQ ID NOS: 60-61; FIG. 6D), Lp4337 (SEQ ID NOS: 62-63; FIG. 6E), Lp328 (SEQ ID NOS: 64-65; FIG. 6E) and 11.21 (SEQ ID NOS: 66-67; FIG. 6E) OMP.

Methods are provided for using Leptospira OMP genes, proteins and antibodies for serological diagnosis techniques.

A plasmid is also provided comprising a recombinant DNA encoding a LP1454, LP1118, LP1939, MCEII, CADF-like1, CADF-like2, CADF-like3, Lp0022, Lp1499, Lp4337, Lp328 or L21 OMP or an epitope of a conserved variant thereof, or an immunologically reactive peptide fragment thereof.

An expression vector is also provided.

Immunogenic epitopes of Leptospira LP1454, LP1118, LP1939, MCEII, CADF-like1, CADF-like2, CADF-like3, Lp0022, Lp1499, Lp4337, Lp328 and L21 OMPs, or an epitope of a conserved variant thereof or an immunologically reactive peptide fragment thereof are also provided.

Methods for using the immunogenic epitope (e.g., a T-cell epitope) as a vaccine (or to improve a vaccine) for mammals, including but not limited to cow, pig, dog, sheep and horse, are provided.

Methods for using the immunogenic epitope (e.g., a T-cell epitope) as a serological diagnostic antigen are also provided. In one aspect, the epitope is specifically recognized by an antibody.

Vaccine formulations for pharmacological administration are also provided.

Vaccine formulations can be DNA vaccine formulations, recombinant vaccine formulations, or T-cell epitope vaccine formulations.

Isolated and purified antibodies to Leptospira OMPs LP1454, LP1118, LP1939, MCEII, CADF-like1, CADF-like2, CADF-like3, Lp0022, Lp1499, Lp4337, Lp328 and L21, or to an immunogenic epitope (e.g., a T-cell epitope), epitope of a conserved variant, or an immunologically reactive peptide fragment thereof are also provided. Such antibodies include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies and epitope-binding fragments of any of the above.

In one embodiment, an isolated and purified antibody of the invention can specifically recognize one or more epitopes of Leptospira LP1454, LP1118, LP1939, MCEII, CADF-like1, CADF-like2, CADF-like3, Lp0022, Lp1499, Lp4337, Lp328 and 121, or epitopes of conserved variants or immunologically reactive peptide fragments thereof.

The isolated and purified antibody to the foregoing Leptospira OMPs can be conjugated to moieties including but not limited to a targeting agent, a label, a carrier, a drug, a toxin and a solid support.

A hybridoma or immortalized cell line is also provided that secretes a monoclonal antibody that can specifically recognize one or more epitopes of Leptospira LP1454, LP1118, LP1939, MCEII, CADF-like1, CADF-like2, CADF-like3, Lp0022, Lp1499, Lp4337, Lp328 and L21, or an epitope of a conserved variant or immunologically reactive peptide fragment thereof.

Methods are provided for producing antibodies that specifically recognize one or more epitopes of Leptospira LP1454, LP1118, LP1939, MCEII, CADF-like1, CADF-like2, CADF-like3, Lp0022, Lp1499, Lp4337, Lp328 and L21, or an epitope of a conserved variant or immunologically reactive peptide fragment thereof.

In one embodiment, the method can comprise immunizing a host animal by injection with an OMP protein or peptide, wherein the OMP protein is an Leptospira LP1454, LP1118, LP1939, MCEII, CADF-like1, CADF-like2, CADF-like3, Lp0022, Lp1499, Lp4337, Lp328 and L21 peptide (e.g., one corresponding to a functional domain of the protein), a truncated polypeptide thereof (in which one or more domains has been deleted), a functional equivalent thereof, or a mutant thereof.

In another embodiment, the method can comprise producing a hybridoma or immortalized cell line that produces an antibody that specifically recognize one or more epitopes of Leptospira LP1454, LP1118, LP1939. MCEII, CADF-like1, CADF-like2, CADF-like3, Lp0022, Lp1499, Lp4337, Lp328 and L21 OMPs, or epitopes of conserved variants thereof or immunologically reactive peptide fragments thereof.

Methods are provided for preventing a Leptospira-related disorder in an animal or human subject in need thereof, comprising administering an amount of a vaccine or an antibody of the invention sufficient to confer immunity to the Leptospira-related disorder to the subject.

A method is provided for treating a Leptospira-related disorder in an animal or human subject in need thereof, comprising administering an amount of an antibody of the invention sufficient to inhibit or decrease the activity of a Leptospira pathogen.

In one embodiment, the method comprises administering an amount of a vaccine of the invention sufficient to inhibit or decrease the activity of the Leptospira pathogen.

In another embodiment, the amount of the vaccine of the invention administered is sufficient to confer immunity to Leptospira infection or a Leptospira-related disorder to the subject.

In another embodiment, the method for treating the Leptospira-related disorder may not involve administration of antibodies or a vaccine of the invention to a subject. For example, antibodies of the invention can be used to kill infectious organisms in vitro where eventual intended use is to combat infection in animals or humans.

A pharmaceutical composition is provided comprising a therapeutically effective amount of an antibody that immunospecifically binds to a Leptospira LP1454, LP1118, LP1939, MCEII, CADF-like1, CADF-like2, CADF-like3, Lp0022, Lp1499, Lp4337, Lp328 and L21 OMP; and a pharmaceutically acceptable carrier.

A pharmaceutical composition is also provided comprising a therapeutically effective amount of a fragment or derivative of an antibody that immunospecifically binds to a Leptospira LP1454, LP1118, LP1939, MCEII, CADF-like1, CADF-like2, CADF-like3, Lp0022, Lp1499, Lp4337, Lp328 and L21 OMP; the fragment or derivative containing the binding domain of the antibody; and a pharmaceutically acceptable carrier.

Methods are provided for assaying for the presence or activity of a LP1454, LP1118, LP1939, MCEII, CADF-like1, CADF-like2, CADF-like3, Lp0022. Lp1499, Lp4337, Lp328 or L21 protein using antibodies to Leptospira outer membrane proteins (OMPs) LP1454, LP1118, LP1939, MCEII, CADF-like1, CADF-like2, CADF-like3, Lp0022, Lp1499, Lp4337 Lp328 or L21 or fragments thereof.

Methods are provided for diagnosing a Leptospira-related disorder in a subject comprising:

providing one or more antibodies to a Leptospira LP1454, LP1118, LP1939, MCEII, CADF-like1, CADF-like2, CADF-like3, Lp0022, Lp1499, Lp4337, Lp328 or L21 protein or a fragment thereof;

collecting a cell or tissue sample, e.g., a blood sample, from the subject,

contacting the sample with the antibodies; and

assaying for the presence or activity of a LP1454, LP1118, LP1939, MCEII, CADF-like1, CADF-like2, CADF-like3, Lp0022, Lp1499, Lp4337, Lp328 or L21 protein in the sample using antibodies to Leptospira LP1454, LP1118, LP1939, MCEII, CADF-like1, CADF-like2, CADF-like3, Lp0022, Lp1499, Lp4337, Lp328 or L21 or fragments thereof;

wherein presence or activity of a LP1454, LP1118, LP1939, MCEII, CADF-like1, CADF-like2, CADF-like3, Lp0022, Lp1499, Lp4337, Lp328 or L21 protein is indicative of the presence of the Leptospira-related disorder in the subject.

Methods are provided for diagnosing or screening for the presence of a Leptospira-related disorder in a subject comprising measuring the level of a LP1454, LP1118, LP1939, MCEII, CADF-like1, CADF-like2, CADF-like3, Lp0022, Lp1499, Lp4337, Lp328 or L21 protein functional activity in a sample derived from the subject, in which a decrease in the level of LP1454, LP1118, LP1939, MCEII, CADF-like1, CADF-like2, CADF-like3, Lp0022, Lp1499, Lp4337, Lp328 or L21 protein or LP1454, LP1118, LP1939, MCEII, CADF-like1, CADF-like2, CADF-like3, Lp0022, Lp1499, Lp4337, Lp328 or L21 protein functional activity in the sample, relative to the level of LP1454, LP1118, LP1939, MCEII, CADF-like1, CADF-like2, CADF-like3, Lp0022, Lp1499, Lp4337, Lp328 or L21 protein or LP1454, LP1118, LP1939, MCEII, CADF-like1, CADF-like2, CADF-like3, Lp0022, Lp1499, Lp4337, Lp328 or L21 I protein functional activity found in an analogous sample not having the disorder, indicates the presence of the disease or disorder or a predisposition for developing the disorder.

Methods are also provided for monitoring therapy using antibodies to Leptospira LP1454, LP1118, LP1939, MCEII, CADF-like1, CADF-like2, CADF-like3, Lp0022, Lp1499, Lp4337, Lp328 or L21 OMPs or fragments thereof.

Methods for using recombinant LP1454, LP1118, LP1939, MCEII, CADF-like1, CADF-like2, CADF-like3, Lp0022, Lp1499, Lp4337, Lp328 or L21 proteins as diagnostic agents are also provided. In one embodiment, a LP1454, LP1118, LP1939, MCEII, CADF-like1, CADF-like2, CADF-like3, Lp0022, Lp1499, Lp4337, Lp328 or L21 protein is used in a kinetic enzyme-linked immunosorbent assay (KELA ELISA).

A kit is provided comprising in one or more containers an antibody of the invention that immunospecifically binds to a Leptospira LP1454, LP1118, LP1939, MCEII, CADF-like1, CADF-like2, CADF-like3, Lp0022, Lp1499, Lp4337, Lp328 or L21 protein.

A kit is provided comprising in one or more containers an expression vector comprising a plasmid, wherein the plasmid comprises a recombinant DNA encoding a LP1454, LP1118, LP1939, MCEII, CADF-like1, CADF-like2, CADF-like3, Lp0022, Lp1499, Lp4337, Lp328 or L21 OMP or an epitope of a conserved variant or an immunologically reactive peptide fragment thereof.

### 4. BRIEF DESCRIPTION OF THE DRAWINGS

The present invention is described herein with reference to the accompanying drawings, in which similar reference characters denote similar elements throughout the several views. It is to be understood that in some instances, various aspects of the invention may be shown exaggerated or enlarged to facilitate an understanding of the invention.

FIG. 1. Homology of hypothesized mammalian cell invasion proteins of Leptospira. The top sequence is MceII, a hypothetical MceI-like protein of L. interrogans serovar Pomona. This protein was renamed “MceII” (Lp0607, SEQ ID NOs: 7-8) to distinguish it from the MceI of L. interrogans serovar Lai (middle sequence). The middle sequence is MceI of L. interrogans serovar Lai (GenBank Accession number AAN49254; SEQ ID NO: 12). The bottom sequence is amino acids 31-293 of Rv1968, a Mce of Mycobacterium tuberculosis CDC1551 (GenBank accession number AE000516, SEQ ID NOS: 13-14). The top sequence. MceII, has approximately 30% homology with the middle sequence, MceI of L. interrogans serovar Lai, and approximately 25-30% homology with the bottom sequence, Rv1968, Mce of Mycobacterium tuberculosis. See Section 6.1.3 for details.

FIGS. 2A-2D. SDS-PAGE and immunoblot analysis of 18 purified recombinant outer membrane proteins of L. interrogans serovar Pomona. FIG. 2A represents purified GST fusion proteins of 18 recombinant proteins of Leptospira in SDS-PAGE gels that were stained with Coomassie brilliant blue R-250. FIGS. 2B, C, and D indicate immunoblots of purified recombinant proteins probes with polyclonal antibodies to GST, bovine sera from experimental infection and natural infection, respectively. Size standards (in kilodaltons) are indicated on the left. Lanes: 1, SDS-PAGE protein molecular weight standard marker (Bio-Rad); 2, Lp1118; 3, Lp1228; 4, Lp1404; 5, MCEI; 6, Lp2268; 7, LigBVT; 8, Lp1332; 9, Lp1965; 10, Lp192; 11, Lp1947; 12, LigCon; 13, LIPL32; 14, Lp1495; 15, Lp1939; 16, MCEII; 17, Lp2471; 18, Lp1931; 19, Lp1454. See Section 6.1.3 for details.

FIGS. 3A-B. Immunogenicity of recombinant outer membrane proteins (OMPs) of Leptospira to bovine sera from experimental and natural infection. ^{a}L. interrogans serovar Grippotyphosa; ^{b}L. interrogans serovar Canicola; ^{c}L. interrogans serovar Copenhagenii; ^{d}L. borgpetersenii serovar Hardjo. 0=negative, 1=weak, 2=strong. See Section 6.1 for details.

FIG. 4. Expression of recombinant OMPs of Leptospira. OMPs were cloned, expressed and purified as recombinant proteins. Purified recombinant OMPs (1.5-2 μg) were subjected to SDS-PAGE electrophoresis. Size standards (in kilodaltons) are indicated on the left. Lanes: 1 and 11, SDS-PAGE protein molecular weight standard marker (Bio-Rad); 2, LP1118; 3, LP1228; 4, LP1404; 5, MCEI; 6, LP1965; 7, LP1332; 9, LP1947; 10, 2471; 11, LP1939; 12, MCEII; 13, LP1932; 14, LP1454. See Section 6.2.3 for details.

FIG. 5. Serum IgG antibody response of rMCE II, rLP1454 and rLP1118-immunized hamsters. See Section 6.2.3 for details.

FIGS. 6A-E. Gene sequences for Leptospira CADF-like1, CADF-like2, CADF-like3, Lp0022, Lp1499, Lp4337, Lp328 and L21 OMPs.

### 5. DETAILED DESCRIPTION OF THE INVENTION

Leptospira outer membrane proteins (OMPs) LP1454, LP1118, LP1939, MCEII, CADF-like1, CADF-like2, CADF-like3, Lp0022, Lp1499, Lp4337, Lp328 or L21 are provided that can serve as tools for developing effective vaccines or diagnostic methods for leptospirosis. The antigenic properties of the Leptospira LP1454, LP1118, LP1939, MCEII, CADF-like1, CADF-like2, CADF-like3, Lp0022, Lp1499, Lp4337, Lp328 or L21 OMPs can be used to create, manufacture or improve vaccines. Vaccines, including but not limited to DNA vaccines, recombinant vaccines, and T cell epitope vaccines based on the foregoing OMPs are further provided. Methods for producing such vaccines are also provided. Also provided are methods for using Leptospira OMP genes, proteins and antibodies for serological diagnosis techniques.

For clarity of disclosure, and not by way of limitation, the detailed description of the invention is divided into the subsections set forth below.

### 5.1 Identification of Immunogenic Proteins from Genome-Derived Putative Outer Membrane Proteins (OMPs) of Leptospira

Immunogenic proteins from genome-derived putative OMPs of Leptospira can be identified using standard methods known in the art. For example, experimentally infected animal sera, e.g., bovine or canine sera can be prepared according to standard methods (e.g., Surujballi, et al. 1997. Development and initial evaluation of an indirect enzyme-linked immunosorbent assay for the detection of Leptospira interrogans serovar hardjo antibodies in bovine sera. Can. J. Vet. Res. 61:260-266).

Infected animals can be identified by their exhibition of clinical symptoms or by a Microscopic Agglutination Test (MAT) using art-known methods. MAT can be carried out as previously described (Cole, J. R., Jr., H. C. Ellinghausen, and H. L. Rubin. 1979, Laboratory diagnosis of leptospirosis of domestic animals. Proc. Annu. Meet. U S Anim. Health Assoc: 189-195) with the whole cell antigens of serovars of interest.

Sera can be collected using standard methods, e.g., from the jugular vein, and stored at −20° C. until use.

Genomic DNA of a Leptospira interrogans serovar, e.g., L. interrogans serovar Pomona, can be prepared using standard methods known in the art, e.g. a DNAeasy kit (Qiagen. Valencia, Calif.). Nucleotide sequences encoding a rLP1454, rLP1118, rLP1939 or rMCEII rCADF-like1, rCADF-like2, rCADF-like3, rLp0022, rLp1499, rLp4337, rLp328 or rL21 OMPs gene or portions thereof may be obtained by PCR amplification of Leptospira genomic DNA. Useful DNA sources include DNA preparations and cloned DNA in DNA libraries. Outer membrane DNA preparations or libraries are particularly preferred.

Genomic DNA can be amplified using standard PCR amplification techniques known in the art. Primers for amplifying and cloning putative outer membrane protein genes, for example, L. interrogans serovar specific sequences, can be designed without the signal sequences using standard methods well known in the art. A selection of primers suitable for use in identifying putative OMP genes is set forth in Example 1, Table 1.

PCR amplification can be carried out using standard methods, e.g., by use of a standard automated PCR thermal cycler and a Taq polymerase such as Accuprime Taq polymerase (Invitrogen, CA). One can choose to synthesize appropriate primers for use in the PCR reactions. It also is possible to vary the stringency of hybridization conditions used in priming the PCR reactions, to allow for greater or lesser degrees of nucleotide sequence similarity between the degenerate primers and the corresponding sequences in the cDNA library. One of ordinary skill in the art will know that the appropriate amplification conditions and parameters depend, in part, on the length and base composition of the primers and that such condition may be determined using standard formulae. Protocols for executing all PCR procedures discussed herein are well known to those skilled in the art, and may be found in references such as Gelfand, 1989, PCR Technology, Principles and Applications for DNA Amplification, H. A. Erlich, ed., Stockton Press, New York; and Current Protocols In Molecular Biology, Vol. 2, Ch. 15, Ausubel et al., eds. 1988, New York, Wiley & Sons, Inc.

The amplification may use, as the 5′-primer (i.e., forward primer), a degenerate oligonucleotide that corresponds to a segment of a known LP1454, LP1118, LP1939, MCEII, CADF-like1, CADF-like2, CADF-like3, Lp0022, Lp1499, Lp4337, Lp328 or L21 amino acid sequence (or other known putative or actual OMP amino acid sequence), preferably from the amino-terminal region. The 3′-primer (i.e., reverse primer) may be a degenerate oligonucleotide that corresponds to a distal segment of the same known LP1454, LP1118, LP1939, MCEII, CADF-like1, CADF-like2, CADF-like3, Lp0022, Lp1499, Lp4337, Lp328 or L21 amino acid sequence (i.e., carboxyl to the sequence that corresponds to the 5′-primer).

For example, the amino acid sequence of the LP1454 (SEQ ID NOS: 1-2; (GenBank/NCBI Entrez Protein Database Accession No. AAN48653; National Center for Biotechnology Information, National Library of Medicine, Bethesda, Md. 20894), LP1118 (SEQ ID NOS: 3-4, GenBank/NCBI Accession No. AAN48316), LP1939 (SEQ ID NOS: 5-6; GenBank/NCBI Accession No. AAN49138) MCEII (Lp0607; SEQ ID NOS: 7-8), CADF-like1, CADF-like2, CADF-like3, Lp0022, Lp1499, Lp4337, Lp328 or L21 protein may be used to design useful 5′ and 3′ primers. Preferably, the primers correspond to segments in the N-terminal of the protein (see Section 6.1, Table 1).

The sequence of the optimal degenerate oligonucleotide probe corresponding to a known amino acid sequence may be determined by standard algorithms known in the art. See for example, Sambrook et al., Molecular Cloning: A Laboratory Manual, 2nd ed., Cold Spring Harbor Laboratory Press, Cold Spring Harbor, NY, Vol 2 (1989).

PCR products can be run, using standard methods known in the art, in a 1% agarose gel and visualized by ethidium bromide staining, PCR products can be eluted from the gel using a gel elution kit (e.g., from Qiagen, Valencia, Calif.).

A PCR-amplified sequence may be molecularly cloned and sequenced. For example, the PCR-amplified can be cloned into a vector, e.g., a TOPO TA vector, as described by the manufacturer (Invitrogen, CA).

The amplified sequence can be utilized as a probe to isolate genomic (or cDNA) clones of a LP1454, LP1118, LP1939, MCEII, CADF-like1, CADF-like2, CADF-like3, Lp0022, Lp11499, Lp4337, Lp328 or L21 gene.

DNA sequencing can be performed with a standard automated nucleic acid sequencer, e.g., an ABI model 377 automated nucleic acid sequencer. Homology searches can be performed using standard searching methods e.g., BLAST, in the NCBI databases (Altschul, S. F., W. Gish, W. Miller, E. W. Myers, and D. J. Lipman. 1990. Basic local alignment search tool. J. Mol. Biol. 215:403-410).

The genomic DNA can be inserted into suitable vectors, including, but not limited to, plasmids, cosmids, bacteriophages lambda or T₄, and yeast artificial chromosome (YAC) or attenuated viruses (such as Herpes virus). See, for example, Sambrook et al., Molecular Cloning: A. Laboratory Manual, 2d ed., Cold Spring Harbor Laboratory Press, Cold Spring Harbor, N.Y. (1989); D. Glover (ed.), DNA Cloning: A Practical Approach, IRL Press, Ltd., Oxford, U.K., Vols. I and II (1985).

The identity of a cloned or amplified putative OMP gene sequence can be verified by comparing the amino acid sequences of its open reading frames with the amino acid sequence of the gene (e.g., Leptospira LP1454, LP1118, LP1939, MCEII, CADF-like1, CADF-like2, CADF-like3, Lp0022, Lp1499, Lp4337, Lp328 or L21 polypeptide whose amino acid sequence is substantially similar to that of a LP1454, LP1118, LP1939, MCEII, CADF-like1, CADF-like2, CADF-like3, Lp0022, Lp1499, Lp4337, Lp328 or L21 protein or polypeptide. The identity of the cloned or amplified OMP gene sequence may be further verified by examining its expression pattern, which should show highly localized expression in the outer membrane of the Leptospira species from which the gene sequence (e.g., LP1454, LP1118, LP1939, MCEII, CADF-like1, CADF-like2, CADF-like3, Lp0022, Lp1499, Lp4337, Lp328 or L21 gene sequence) was isolated.

Homology searches can be performed using standard search methods in the NCBI database and BLAST (Altschul, S. F., W. Gish, W. Miller, E. W. Myers, and D. J. Lipman. 1990. Basic local alignment search tool. J. Mol. Biol. 215:403-410).

Although the polynucleotide sequences encoding LP1454, LP1118, LP1939, MCEII, CADF-like1, CADF-like2, CADF-like3, Lp0022, Lp1499, Lp4337, Lp328 or L21 OMP proteins described herein can be derived L. interrogans serovar Pomona, the sequences can be derived from another organism, derived from any recombinant source, synthesized in vitro or by chemical synthesis. The nucleotides in the polynucleotide sequences may be DNA or RNA and may exist in a double-stranded, single-stranded or partially double-stranded form.

Nucleic acids can also be prepared by any conventional means typically used to prepare nucleic acids in large quantity. For example, DNAs and RNAs may be chemically synthesized using commercially available reagents and synthesizers by methods that are well-known in the art (see, e.g., Gait, 1985, Oligonucleotide Synthesis: A Practical Approach, IRL Press, Oxford, England). RNAs may be produce in high yield via in vitro transcription using plasmids such as SP65 (Promega Corporation, Madison, Wis.).

The nucleic acids may be purified by any suitable means, as are well known in the art. For example, the nucleic acids can be purified by reverse phase or ion exchange HPLC, size exclusion chromatography or gel electrophoresis. Of course, the skilled artisan will recognize that the method of purification will depend in part on the size of the DNA to be purified.

### 5.2 Expression Systems

Using standard methods, inserts can be sub-cloned into expression vectors, e.g., pGEX-KG (Stratagene), pRSETA (InVitroGen, CA), attenuated herpes virus vector, etc., using standard methods known in the art, and transformed into E. coli (BL21 DE3 or CQ21).

In one embodiment, a vector capable of expressing a recombinant DNA is provided. The recombinant DNA can be selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above,
   and wherein the recombinant DNA is inserted into the vector such that a recombinant protein is expressed when the vector is provided in an appropriate host.

In bacterial systems a number of expression vectors may be advantageously selected depending upon the use intended for the expressed OMPs, such as LP1454, LP1118, LP1939, MCEII, CADF-like1, CADF-like2, CADF-like3, Lp0022, Lp1499, Lp4337, Lp328 or L21 product.

For example, when large quantities of LP1454, LP1118, LP1939, MCEII, CADF-like1, CADF-like2, CADF-like3, Lp0022, Lp1499, Lp4337, Lp328 or L21 protein are to be produced for the generation of antibodies, screening peptide libraries or formulating pharmaceutical compositions, vectors that direct the expression of high levels of fusion protein products that are readily purified may be desirable. Such vectors include but are not limited to the E. coli expression vector pUR278 (Ruther et al., 1983, EMBO J. 2:1791), in which the LP1454, LP1118, LP1939, MCEII, CADF-like1, CADF-like2, CADF-like3, Lp0022, Lp1499, Lp4337, Lp328 or L21 coding sequence may be ligated into the vector in frame with the lacZ coding region so that a hybrid protein is produced; pIN vectors (Inouye & Inouye, 1985, Nucleic acids Res. 13:3101-3109; Van Heeke & Schuster, 1989, J. Biol. Chem. 264:5503-5509); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned polypeptide of interest can be released from the GST moiety.

In one such embodiment of a bacterial system, full length DNA sequences are appended with in-frame BamHI sites at the amino terminus and EcoRI sites at the carboxyl terminus using standard PCR methodologies (Innis et al. 1990, supra) and ligated into the pGEX-2TK vector (Pharmacia, Uppsala, Sweden). The resulting cDNA construct contains a kinase recognition site at the amino terminus for radioactive labeling and glutathione S-transferase sequences at the carboxyl terminus for affinity purification (Nilsson, et al., 1985, EMBO J. 4: 1075; Zabeau and Stanley, 1982, EMBO J. 1: 1217).

The recombinant constructs of the present invention may include a selectable marker for propagation of the construct. For example, a construct to be propagated in bacteria preferably contains an antibiotic resistance gene, such as one that confers resistance to kanamycin, tetracycline, streptomycin or chloramphenicol. Suitable vectors for propagating the construct include plasmids, cosmids, bacteriophages or viruses, to name but a few.

In addition, the recombinant constructs may include cell-expressible, selectable or screenable marker genes for isolating, identifying or tracking cells transformed by these constructs. Selectable markers include, but are not limited to, genes that encode visible markers such as green fluorescent protein (GFP) or those that confer antibiotic resistance, (e.g. resistance to kanamycin or hygromycin).

In yeast, a number of vectors containing constitutive or inducible promoters may be used (Current Protocols in Molecular Biology, Vol. 2, 1988, Ed. Ausubel et al., Greene Publish. Assoc. & Wiley Interscience, Ch. 13; Grant et al., 1987, Expression and Secretion Vectors for Yeast, in Methods in Enzymology, Eds. Wu & Grossman, 1987, Acad. Press, N.Y., Vol. 153, pp. 516-544; Glover, 1986, DNA Cloning, Vol. II, IRL Press, Wash., D.C., Ch. 3; and Bitter, 1987, Heterologous Gene Expression in Yeast, Methods in Enzymology, Eds. Berger & Kimmel Acad. Press, N.Y., Vol. 152, pp. 673-684; and The Molecular Biology of the Yeast Saccharomyces, 1982, Eds. Strathern et al., Cold Spring Harbor Press, Vols. I and II).

Another alternative expression system that can be used to express LP1454, LP1118, LP1939, MCEII, CADF-like1, CADF-like2, CADF-like3, Lp0022, Lp1499, Lp4337, Lp328 or L21 is an insect system. In one such system, Autographa californica nuclear polyhedrosis virus (AcNPV) is used as a vector to express foreign genes. The virus grows in Spodoptera frugiperda cells. The rLP1454, rLP1118, rLP1939, rMCEII, rCADF-like1, rCADF-like2, rCADF-like3, rLp0022, rLp1499, rLp4337, rLp328 or rL21 coding sequence may be cloned into non-essential regions (for example the polyhedron gene) of the virus and placed under control of an AcNPV promoter (for example the polyhedron promoter). Successful insertion of the rLP1454, rLP1118, rLP1939, rMCEII, rCADF-like1, rCADF-like2, rCADF-like3, rLp0022, rLp1499, rLp4337, rLp328 or rL21 coding sequence will result in inactivation of the polyhedron gene and production of non-occluded recombinant virus (i.e., virus lacking the proteinaceous coat coded for by the polyhedron gene). These recombinant viruses are then used to infect Spodoptera frugiperda cells in which the inserted gene is expressed (e.g., see Smith et al., 1983, J. Viol. 46:584; Smith, U.S. Pat. No. 4,215,051).

In mammalian host cells, a number of viral based expression systems may be utilized, such as an attenuated herpes virus vector or an adenovirus.

In cases where an adenovirus is used as an expression vector, the LP1454, LP1118, LP1939, MCEII, CADF-like1, CADF-like2, CADF-like3, Lp0022, Lp1499, Lp4337, Lp328 or L21 coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing LP1454, LP1118, LP1939, MCEII, CADF-like1, CADF-like2, CADF-like3, Lp0022, Lp1499, Lp4337, Lp328 or L21 in infected hosts (e.g., See Logan & Shenk, 1984, Proc. Natl. Acad. Sci. USA 81:3655-3659). Alternatively, a vector derived from vaccinia virus can be used, which would typically make use of the vaccinia 7.5K promoter (See, e.g., Mackett et al., 1982, Proc Natl. Acad. Sci. USA 79:7415-7419; Mackett et al., 1984, J. Virol. 49:857-864; Panicali et al., 1982, Proc. Natl. Acad. Sci. USA 79:4927-4931). Regulatable expression vectors such as the tetracycline repressible vectors may also be used to express the coding sequences in a controlled fashion.

Specific initiation signals may also be required for efficient translation of inserted LP1454, LP1118, LP1939, MCEII, CADF-like1, CADF-like2, CADF-like3, Lp0022, Lp1499, Lp4337, Lp328 or L21 coding sequences. These signals include the ATG initiation codon and adjacent sequences. In cases where the entire LP14548, LP1118, LP1939, MCEII, CADF-like1, CADF-like2, CADF-like3, Lp0022, Lp1499, Lp4337, Lp328 or L21 gene, including its own initiation codon and adjacent sequences, is inserted into the appropriate expression vector, no additional translational control signals may be needed. However, in cases where only a portion of the LP1454, LP1118, LP1939, MCEII, CADF-like1, CADF-like2, CADF-like3, Lp0022, Lp1499, Lp4337, Lp328 or L21 coding sequence is inserted, exogenous translational control signals, including the ATG initiation codon, must be provided. Furthermore, the initiation codon must be in phase with the reading frame of the LP1454, LP1118, LP1939, MCEII, CADF-like1, CADF-like2, CADF-like3, Lp0022, Lp1499, Lp4337, Lp328 or L21 coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al. 1987, Methods in Enzymol. 153:516-544).

In addition, a host cell strain can be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used.

For long-term, high-yield production of recombinant proteins, stable expression is preferred. For example, cell lines that stably express the LP1454, LP1118, LP1939, MCEII, CADF-like1, CADF-like2, CADF-like3, Lp0022, Lp1499, Lp4337, Lp328 or L21 proteins may be engineered. Rather than using expression vectors that contain viral origins of replication, host cells can be transformed with the LP1454, LP1118, LP1939, MCEII, CADF-like1, CADF-like2, CADF-like3, Lp0022, Lp1499, Lp4337, Lp328 or L21 coding sequence controlled by appropriate expression control elements (e.g., promoter and/or enhancer sequences, transcription terminators, polyadenylation sites, etc.), and a selectable marker. Following the introduction of foreign DNA, genetically engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the LP1454, LP1118, LP1939, MCEII, CADF-like1, CADF-like2, CADF-like3, Lp0022, Lp1499, Lp4337, Lp328 or L21 protein. Such engineered cell lines are particularly useful in screening for molecules or drugs that affect LP1454, LP1118, LP1939, MCEII, CADF-like1, CADF-like2, CADF-like3, Lp0022, Lp1499, Lp4337, Lp328 or L21 function.

A number of selection systems may be used, including but not limited to, the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk⁻, hgprt⁻ or aprt⁻ cells, respectively. Also, antimetabolite resistance can be used as the basis of selection for dhfr, which confers resistance to methotrexate (Wigler, et al., 1980, Proc. Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes. Additional selectable genes include trpB, which allows cells to utilize indole in place of tryptophan; hisD, which allows cells to utilize histinol in place of histidine (Hartman & Mulligan, 1988, Proc. Natl. Acad. Sci. USA 85:8047); ODC (ornithine decarboxylase), which confers resistance to the ornithine decarboxylase inhibitor, 2-(difluoromethyl)-DL-orithine, DFMO) (McConlogue L., 1987, In: Current Communications in Molecular Biology, Cold Spring Harbor Laboratory ed.) and glutamine synthetase (Bebbington et al., 1992, Biotech 10:169).

The expression characteristics of an endogenous LP1454, LP1118, LP1939, MCEII, CADF-like1, CADF-like2, CADF-like3, Lp0022, Lp1499, Lp4337, Lp328 or L21 gene within a cell line, plant or microorganism may be modified by inserting a heterologous DNA regulatory element into the genome of a stable cell line, plant or cloned microorganism such that the inserted regulatory element is operatively linked with the endogenous LP1454, LP1118, LP1939, MCEII, CADF-like1, CADF-like2, CADF-like3, Lp0022, Lp1499, Lp4337, Lp328 or L21 gene. For example, an endogenous gene that is normally “transcriptionally silent”, i.e., an LP1454, LP1118, LP1939, MCEII, CADF-like1, CADF-like2, CADF-like3, Lp0022, Lp1499, Lp4337, Lp328 or L21 gene that is normally not expressed, or is expressed only at very low levels in a cell line, plant or microorganism, may be activated by inserting a regulatory element that is capable of promoting the expression of a normally expressed gene product in that cell line, plant or microorganism. Alternatively, a transcriptionally silent, endogenous gene may be activated by insertion of a promiscuous regulatory element that works across cell types.

A heterologous regulatory element may be inserted into a stable cell line, plant or cloned microorganism, such that it is operatively linked with an endogenous LP11454, LP1118, LP1939, MCEII, CADF-like1, CADF-like2, CADF-like3, Lp0022, Lp1499, Lp4337, Lp328 or L21 gene, using techniques that are well known to those of skill in the art, such as targeted homologous recombination (e.g., in Chappel, U.S. Pat. No. 5,272,071; PCT publication No. WO 91/06667, published May 16, 1991).

### 5.3 Production of Leptospira Outer Membrane Proteins and Polypeptides

While leptospiral OMP polypeptides and peptides, such as LP1454, LP1118, LP1939, MCEII, CADF-like1, CADF-like2, CADF-like3, Lp0022, Lp1499, Lp4337, LP328 or L21 polypeptides and peptides, can be chemically synthesized (e.g., see Creighton, 1983, Proteins: Structures and Molecular Principles, W.H. Freeman & Co., N.Y.) large polypeptides derived from LP1454, LP1118, LP1939, MCEII, CADF-like1, CADF-like2, CADF-like3, Lp0022, Lp1499, Lp4337, Lp328 or L21 and full length proteins may advantageously be produced by recombinant DNA technology using techniques well known to those skilled in the art for expressing nucleic acid sequences.

Methods that are well known to those skilled in the art can be used to construct expression vectors containing the LP1454, LP1118, LP1939, MCEII, CADF-like1, CADF-like2, CADF-like3, Lp0022, Lp1499, Lp4337, Lp328 or L21 coding sequence and appropriate transcriptional/translational control signals. These methods include in vitro recombinant DNA techniques, synthetic techniques and in vivo recombination/genetic recombination. (See, for example, the techniques described in Sambrook et al., 1989, Molecular Cloning A Laboratory Manual, Cold Spring Harbor Laboratory, N.Y. and Ausubel et al., 1989, Current Protocols in Molecular Biology, Greene Publishing Associates and Wiley Interscience, N.Y.). RNA capable of encoding LP1454, LP1118, LP1939, MCEII, CADF-like1, CADF-like2, CADF-like3, Lp0022, Lp1499, Lp4337, Lp328 or L21 polypeptide may also be chemically synthesized (Gait, ed., 1984, Oligonucleotide Synthesis, IRL Press, Oxford).

A variety of host-expression vector systems may be utilized to express the gene products. Such host-expression systems represent vehicles by which the gene products of interest may be produced and subsequently recovered and/or purified from the culture or organism (using purification methods well known to those skilled in the art), but also represent cells that may, when transformed or transfected with the appropriate nucleotide coding sequences, exhibit the LP1454, LP1118, LP1939, MCEII, CADF-like1, CADF-like2, CADF-like3, Lp0022, Lp1499, Lp4337, Lp328 or L21 protein in situ.

These include, but are not limited to, microorganisms such as bacteria (e.g., E. coli, B. subtilis) transformed with recombinant bacteriophage DNA, plasmid DNA or cosmid DNA expression vectors containing LP1454, LP1118, LP1939, MCEII, CADF-like1, CADF-like2, CADF-like3, Lp0022, Lp1499, Lp4337, Lp328 or L21 protein coding sequences; yeast (e.g., Saccharomyces, Pichia) transformed with recombinant yeast expression vectors containing the protein coding sequences; insect cell systems infected with recombinant virus expression vectors (e.g., baculovirus) containing the protein coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing the protein coding sequences; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter; the cytomegalovirus promoter/enhancer; etc.).

Depending on the host/vector system utilized, any of a number of suitable transcription and translation elements, including constitutive and inducible promoters, may be used in the expression vector. For example, when cloning in bacterial systems, inducible promoters such as pL of bacteriophage λ, plac, ptrp, ptac (ptrp-lac hybrid promoter; cytomegalovirus promoter) and the like may be used; when cloning in insect cell systems, promoters such as the baculovirus polyhedron promoter may be used; when cloning in plant cell systems, promoters derived from the genome of plant cells (e.g., heat shock promoters; the promoter for the small subunit of RUBISCO; the promoter for the chlorophyll f/u binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the L1454, LP1118, LP1939 MCEII, CADF-like1, CADF-like2, CADF-like3, Lp0022, Lp1499, Lp4337, Lp328 or L21 coding sequence, SV40-, BPV- and EBV-based vectors may be used with an appropriate selectable marker.

A protein region can be considered “homologous” to a second protein region when the amino acid sequence of the first region is at least 30%, 40%, 50%, 60%, 70% 75%, 75%, 80%, 90%, or 95% identical, when compared to any sequence in the second region of an equal number of amino acids as the number contained in the first region or when compared to an aligned sequence of the second region that has been aligned by a computer homology program known in the art e.g., the BLAST program described above.

### 5.4 In Situ Expression of Outer Membrane Proteins

rLP1454, rLP1118, rLP1939, rMCEII, rCADF-like1, rCADF-like2, rCADF-like3, rLp0022, rLp1499, rLp4337, rLp328 and rL21 genes show highly localized expression in the Leptospira outer membrane. Such expression patterns may be ascertained by using standard art-known methods such as Northern hybridization, in situ hybridizations using antisense probes isolation of the (OMPs from the outer membranes, or by standard methods of immunogold labeling (see, e.g., Matsunaga J, et al. Pathogenic Leptospira species express surface-exposed proteins belonging to the bacterial immunoglobulin superfamily. Mol Microbiol. 2003 August; 49(4):929-45.

### 5.5 Protein Identification and Purification

Once a recombinant protein is expressed, it can be identified by assays based on the physical or functional properties of the product, including radioactive labeling of the product followed by analysis by gel electrophoresis, radioimmunoassay, ELISA, bioassays, etc.

Once the encoded protein is identified, it may be isolated and purified by standard methods including chromatography (e.g., high performance liquid chromatography, ion exchange, affinity, and sizing column chromatography), centrifugation, differential solubility, or by any other standard technique for the purification of proteins. The actual conditions used will depend, in part, on factors such as net charge, hydrophobicity, hydrophilicity, etc., and will be apparent to those having skill in the art. The functional properties may be evaluated using any suitable assay, e.g. an assay for the ability to alter root formation. For the practice of the present invention, it is preferred that the polypeptide is at least 80% purified from other proteins. It is more preferred that they are at least 90% purified. For in vivo administration, it is preferred that it is greater than 95% purified, and more preferably greater than 99%. For example, recombinant proteins can be expressed and purified as follows. Exponentially growing cultures (OD₆₀₀=0.6) of E. coli (BL21 DE3) or CQ21 harboring recombinant plasmids can be induced to synthesize fusion proteins by standard methods using 1 mM isopropyl-β-D-thiogalactopyranoside (IPTG; Sigma, St. Louis, Mo.). For each recombinant protein, a pilot experiment can be performed with 3 mL culture volume

The molecular weight of the expressed recombinant protein can be checked by SDS-PAGE analysis. SDS-PAGE can be performed using standard methods (e.g., Chang, Y. F., D. P. Ma, J, Shi, and M. M. Chengappa. 1993. Molecular characterization of a leukotoxin gene from a Pasteurella haemolytica-like organism, encoding a new member of the RTX toxin family. Infect. Immun. 61:2089-2095). Recombinant proteins can be loaded into SDS-PAGE gel and stained with Coomassie brilliant blue R-250 followed by destaining.

Mass cultivation can be performed according to standard methods, e.g., in 500 mL of LB broth with ampicillin (50 μg/mL). Bacteria can be harvested by standard methods, e.g., by centrifugation at 5,000 RPM for 30 min. The cell pellets can then be washed and suspended in PBS followed by passing through a French pressure cell (e.g., American Instrument).

Lysates can be centrifuged using standard methods at 8,000×g for 30 min and the inclusion bodies washed and purified using standard methods (e.g. Palaniappan, et al. 2004. Expression of leptospiral immunoglobulin-like protein by Leptospira interrogans and evaluation of its diagnostic potential in a kinetic ELISA. J. Med. Microbiol. 53:975-984, Palaniappan, et al. 2002. Cloning and molecular characterization of an immunogenic LigA protein of Leptospira interrogans. Infect. Immun. 70:5924-5930) with model prep cell (BioRad, CA).

Purified protein can be isolated by dialysis at 4° C. with PBS by changing PBS for at least 4 times. The purified protein can be concentrated and lyophilized (Palaniappan, R. U., Y. F. Chang, F. Hassan, S. P. McDonough, M. Pough, S. C. Barr, K. W. Simpson, H. O. Mohammed, S. Shin, P. McDonough. R. L. Zuerner, J. Qu, and B. Roe. 2004. Expression of leptospiral immunoglobulin-like protein by Leptospira interrogans and evaluation of its diagnostic potential in a kinetic ELISA. J. Med. Microbiol. 53:975-984). Lyophilized protein can be suspended with sterile PBS and quantified by the Bradford assay (Bio-Rad).

Recombinant proteins can be separated using standard methods, e.g., by running SDS-PAGE followed by transfer onto nitrocellulose membrane (Schleicher & Schuell, Keene, N.H.) in a Trans Blot Cell (Bio-Rad Laboratories, Hercules, Calif.) and immunoblotting (e.g., western blotting). For example, after transfer, the membrane can be blocked with TBS plus 1% bovine serum albumin (BSA) for 1 h. Antisera can then be diluted in TBS-BSA and incubated with the blot for 1 h. After three washings in TBS plus 0.1% Tween 20, the blot can be incubated for 1 h with 1:2,000 Goat anti-bovine IgG conjugated with alkaline phosphatase (KPL). The blot can be washed three times as described above and developed with NBT/BCIP as described by Chang et al. (1995, Recombinant OspA protects dogs against infection and disease caused by Borrelia burgdorferi. Infect. Immun. 63:3543-3549).

The recombinant protein expression can also be assessed immunologically by immunoassays such as radioimmuno-precipitation, enzyme-linked immunoassays and the like. This can be achieved by using an anti-OMP antibody, e.g., an anti-LP1454, LP1118, LP1939, MCEII, CADF-like1, CADF-like2, CADF-like3, Lp0022, Lp499, Lp4337, Lp328 or L21 protein antibodies.

Expression of the protein product can also be assessed using analytical techniques such as amino acid sequencing, which can be accomplished by means of, for example, Edman degradation or tandem mass spectroscopy, or by analysis of the masses of peptides generated by partial hydrolysis of the protein product using mass spectroscopy. In the identification of a protein of interest (e.g., a LP1454, LP1118, LP1939, MCEII, CADF-like1, CADF-like2, CADF-like3, Lp0022, Lp1499, Lp4337, Lp328 or L21 protein) by mass spectroscopy, it will often be desirable to separate the protein of interest from other protein constituents of the cell by means of two-dimensional gel electrophoresis, partially hydrolyze the isolated protein using an amino acid specific protease (e.g., Lys-C, trypsin), and then determine the mass of the resulting peptide fragments using mass spectroscopy.

Determination of peptide mass can then be used to identify the protein as LP1454, LP18, LP1939, MCEII, CADF-like1, CADF-like2, CADF-like3, Lp0022, Lp1499, Lp4337, Lp328 or L21, or a variant thereof, using a database of the predicted masses of protein proteolysis products and analysis software such as Protein Prospector, which is publicly available on the internet at http://prospector.ucsf.edu.

### 5.6 Antibodies to Outer Membrane Proteins and Polypeptides

Antibodies that specifically recognize one or more epitopes of a Leptospira outer membrane protein, such as LP1454, LP1118, LP1939, MCEII, CADF-like1, CADF-like2, CADF-like3, Lp0022, Lp1499, Lp4337, Lp328 or L21, or epitopes of conserved variants or peptide fragments thereof are also provided. Such antibodies include, but are not limited to, polyclonal antibodies, monoclonal antibodies (mAbs), human, humanized or chimeric antibodies, single chain antibodies, Fab fragments, F(ab′)₂ fragments, fragments produced by a Fab expression library, anti-idiotypic (anti-Id) antibodies and epitope-binding fragments of any of the above.

A method for producing an antibody is also provided wherein the method comprises: (a) providing a microorganism in a culture containing a DNA encoding a fusion polypeptide comprising at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21, linked to a polypeptide that facilitates isolation of the fusion polypeptide; (b) culturing the microorganism in a culture to produce the fusion polypeptide; (c) isolating the fusion polypeptide; (d) producing the antibody from the polypeptide. In a preferred embodiment, the polypeptide is removed from the antigen portion of the fusion polypeptide.

A method for producing a monoclonal antibody is also provided comprising: (a) providing a microorganism in a culture containing a DNA encoding a fusion polypeptide comprising a at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21, linked to a polypeptide that facilitates isolation of the fusion polypeptide; (b) culturing the microorganism in a culture to produce the fusion polypeptide; (c) isolating the fusion polypeptide; and (d) producing the monoclonal antibody from the polypeptide. Preferably, the polypeptide is removed from the antigen portion of the fusion polypeptide.

In one embodiment, an isolated and purified antibody of the invention can specifically recognize one or more epitopes of a Leptospira LP1454, LP1118, LP1939, MCEII, CADF-like1, CADF-like2, CADF-like3, Lp0022, Lp1499. Lp4337, Lp328 or L21 OMP, or epitopes of conserved variants or immunologically reactive peptide fragments thereof.

The isolated and purified antibody to the Leptospira LP1454, LP1118, LP1939, MCEII, CADF-like1, CADF-like2, CADF-like3, Lp0022, Lp1499, Lp4337, Lp328 or L21 OMP can be conjugated to moieties including but not limited to a targeting agent, a label, a carrier, a drug, a toxin and a solid support.

For the production of antibodies, various host animals may be immunized by injection with the OMP protein or peptide of interest, such as an LP1454, LP1118, LP1939, MCEII, CADF-like1, CADF-like2, CADF-like3, Lp0022, Lp1499, Lp4337, Lp328 or L21 peptide (e.g., one corresponding to a functional domain of the protein), a truncated polypeptide (in which one or more domains has been deleted), functional equivalents of the protein or mutants of the protein. Such proteins, polypeptides, peptides or fusion proteins can be prepared and obtained as described above. Host animals may include, but are not limited to, dogs, cows, sheep, horses, rabbits, rats and mice, to name but a few.

Various adjuvants may be used to increase the immunological response, depending on the host species, including, but not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol and potentially useful human adjuvants such as BCC (bacille Calmette-Guerin) and Corynebacterium parvum.

Polyclonal antibodies are heterogeneous populations of antibody molecules derived from the sera of the immunized animals.

A hybridoma or immortalized cell line is also provided that secretes a monoclonal antibody of the invention. A hybridoma or immortalized cell line can be produced using methods known in the art that produces an antibody that specifically recognize one or more epitopes of Leptospira LP1454, LP1118, LP1939, MCEII, CADF-like1, CADF-like2, CADF-like3, Lp0022, Lp1499, Lp4337, Lp328 or L21 OMPs, or immunologically reactive peptide fragments thereof.

Monoclonal antibodies to Leptospira outer membrane proteins or polypeptides, such as LP1454, LP1118, LP1939, MCEII, CADF-like1, CADF-like2, CADF-like3, Lp0022, Lp1499, Lp4337, Lp328 or L21 proteins or polypeptides, may be prepared by using any technique that provides for the production of antibody molecules by continuous cell lines in culture. These include but are not limited to the hybridoma technique originally described by Kohler and Milstein, (Nature, 1975, 256:495-497), the human B-cell hybridoma technique (Kosbor et al., 1983, Immunology Today, 4:72; Cote et al., 1983, Proc. Natl. Acad. Sci., 80:2026-2030) and the EBV-hybridoma technique (Cole et al., 98, 1985, Monoclonal Antibodies and Cancer Therapy, Alan R. Liss, Inc., pp, 77-96). Such antibodies may be of any immunoglobulin class including, but not limited to, IgG, IgM, IgE, IgA, IgD and any subclass thereof. The hybridoma producing the monoclonal antibodies of this invention may be cultivated in vitro or in vivo.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety). Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987). J. Immunol. 139:3521-3526; Sun et al. (1987) Proc. Natl. Acad. Sci. USA 84:214-218; Nishimura et al., (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

With respect to treatment of a particular species, such as a cow (or dog), completely bovine (or canine) antibodies are particularly desirable for therapeutic treatment. Such antibodies can be produced, for example, using transgenic mice that are incapable of expressing endogenous immunoglobulin heavy and light chains genes, but that can express bovine (or canine) heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol, 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix. Inc. (Fremont, Calif.), can be engaged to provide human antibodies directed against a selected antigen using technology similar to that described above.

Such completely species-specific antibodies that recognize a selected epitope can be generated using a technique referred to as “guided selection.” In this approach a selected non-human monoclonal antibody, e.g., a mouse antibody, is used to guide the selection of a completely species-specific, e.g., bovine or canine antibody recognizing the same epitope. (Jespers et al. (1994) Bio/technology 12:899-903).

Alternatively, techniques described for the production of single chain antibodies (U.S. Pat. No. 4,946,778; Bird, 1988, Science 242:423-426; Huston et al., 1988, Proc. Natl. Acad. Sci. USA 85:5879-5883; and Ward et al., 1989, Nature 334:544-546) can be adapted to produce single chain antibodies against LP1454, LP1118, LP1939, MCEII, CADF-like1, CADF-like2, CADF-like3, Lp0022, Lp1499, Lp4337, Lp328 or L21 proteins or polypeptides. Single chain antibodies are formed by linking the heavy and light chain fragments of the Fv region via an amino acid bridge, resulting in a single chain polypeptide.

Antibody fragments that recognize specific epitopes may be generated by known techniques. For example, such fragments include, but are not limited to: the F(ab′)₂ fragments, which can be produced by pepsin digestion of the antibody molecule and the Fab fragments, which can be generated by reducing the disulfide bridges of the F(ab′)₂ fragments. Alternatively, Fab expression libraries may be constructed (Huse et al., 1989, Science, 246:1275-1281) to allow rapid and easy identification of monoclonal Fab fragments with the desired specificity.

Antibodies to a LP1454, LP1118, LP1939, MCEII, CADF-like1, CADF-like2, CADF-like3, Lp0022, Lp1499, Lp4337, Lp328 or L21 protein and/or polypeptide can, in turn, be utilized to generate anti-idiotype antibodies that “mimic” LP1454, LP1118, LP1939, MCEII, CADF-like1, CADF-like2, CADF-like3, Lp0022, Lp1499, Lp4337, Lp328 or L21, using techniques well known to those skilled in the art, (See, e.g., Greenspan & Bona, 1993, FASEB J 7(5):437-444; and Nissinoff, 1991, J. Immunol. 147(8):2429-2438).

### 5.7 Serological Diagnosis Techniques

### KELA ELISA, Western Blotting (Immunoblots) and PCR

Recombinant LP1454, LP1118, LP1939, MCEII, CADF-like1, CADF-like2, CADF-like3, Lp0022, Lp1499, Lp4337, Lp328 or L21 proteins used as diagnostic agents are also provided. One diagnostic technique in which the foregoing OMPs can be used is kinetic enzyme-linked immunosorbent assay (KELA ELISA) (Appel M J, et al. Experimental Lyme disease in dogs produces arthritis and persistent infection, J Infect Dis. 1993 March; 167(3):651-64; Chang Y F, et al., Recombinant OspA protects dogs against infection and disease caused by Borrelia burgdorferi. Infect Immun. 1995 September; 63(9):3543-9). KELA measures the levels of serum antibodies to Leptospira that are present in a serum sample. In this diagnostic technique, diluted serum (1:100 dilution) is added to duplicate wells in microtiter plates that contain antigens of the OMP of interest.

Antigens can be expressed and purified using methods known in the art, for example, the antigens can be expressed in E. coli, as described herein in Section 6.

The bound antibodies are then detected by using secondary antibodies, e.g., goat anti-canine, bovine, equine or porcine antibodies of heavy and light chain specificity conjugated to horseradish peroxidase (HRP). Color development is seen and measured using the chromogen tetramethylbenzidine with H₂O₂ as a substrate, which is measured kinetically and expressed as the slope of the reaction rate between the enzyme and substrate solution. Each unit of slope is designated as a KELA unit. The cutoff point between positive and negative samples is then confirmed by Western blotting against a French-pressed sample, e.g. Leptospira sample.

Western blot analysis can be carried using standard art-known methods (e.g., Appel M. J., et al. Experimental Lyme disease in dogs produces arthritis and persistent infection, J Infect Dis, 1993 March; 167(3):651-64; Chang Y T, et al., Recombinant OspA protects dogs against infection and disease caused by Borrelia burgdorferi, Infect Immun. 1995 September; 63(9):3543-9.). Recombinant LP1454, LP1118, LP1939, MCEII, CADF-like1, CADF-like2, CADF-like3, Lp0022, Lp1499 Lp4337, Lp328 or L21 can be used as antigens and are subjected to sodium dodecyl sulfate-polyacrylamide gel electrophoresis (SDS-PAGE). Western blot analysis is performed in a miniblotter. Test sera from experimental animals are used as a first antibody, followed by, for example, a goat anti-canine, bovine, equine, canine or porcine IgG conjugated to HRP as a second antibody. Bands are developed by using substrates, such as 24 μg of 4-chloro-1-naphthol in 8 ml of methyl alcohol, 40 ml of Tris-buffer solution, and 24 μl of 30% H₂O₂.

Another diagnostic technique recombinant LP1454, LP1118, LP1939, MCEII, CADF-like1, CADF-like2, CADF-like3, Lp0022, Lp1499, Lp4337, Lp328 or L21 proteins can be used for is PCR diagnosis. The DNA from biopsy samples of kidney or from post-mortem tissues or blood are extracted by techniques known in the art (Palaniappan R U, et al. Evaluation of lig-based conventional and real time PCR for the detection of pathogenic leptospires. Mol Cell Probes. 2005 April; 19(2): 111-7. Epub 2004 Dec. 15; Chang Y F, et al. Recombinant OspA protects dogs against infection and disease caused by Borrelia burgdorferi. Infect Immun. 1995 September; 63(9):3543-9). To prevent contamination of the mixtures and samples, DNA extraction, amplification, and detection of PCR products can be performed at different locations, e.g., in different rooms. Amplification of rLP1454, rLP1118, rLP1939, rMCEII, rCADF-like1, rCADF-like2, rCADF-like3, rLp0022, rLp1499, rLp4337, rLp328 or rL21-specific target sequences can be carried out in a 50-μl reaction mixture. As a positive control, Leptospira genomic DNA can be used. As a negative control, distilled water can be used.

The reaction mixture can be put through cycle of DNA amplification, e.g. 40 cycles of amplification using an automated DNA thermal cycler. Each cycle involves heating the reaction mixture to 94° C. from 1 minute, to cause the DNA to denature; cooling of the reaction mixture to 69° C. for 1 minute, to allow the primers to anneal; and then heating the reaction mixture to 72° C. for 2 minutes, to allow primer extension to occur. Gel electrophoresis on a 1.5% agarose gel is done in order to get visualization of the PCR amplification products.

### 5.8 Pharmaceutical Compositions and Vaccines

Pharmaceutical compositions and vaccines are also provided. Such compositions can comprise a therapeutically effective amount of a pharmaceutical composition or vaccine of the invention, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the Therapeutic, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. The composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In one embodiment, a pharmaceutical composition can comprise a therapeutically effective amount of an antibody that immunospecifically binds to a Leptospira LP1454, LP1118, LP1939, MCEII, CADF-like1, CADF-like2, CADF-like3, Lp0022, Lp1499, Lp4337, Lp328 or L21 protein; and a pharmaceutically acceptable carrier.

In another embodiment, a pharmaceutical composition can comprise a therapeutically effective amount of a fragment or derivative of an antibody that immunospecifically binds to a Leptospira LP1454, LP1118, LP1939 MCEII, CADF-like1, CADF-like2, CADF-like3, Lp0022, Lp14997, Lp337 Lp328 or L21 protein; the fragment or derivative containing the binding domain of the antibody; and a pharmaceutically acceptable carrier.

In another embodiment, a pharmaceutical composition can comprise a therapeutically effective amount of a vaccine of the invention; and a pharmaceutically acceptable carrier.

Vaccine formulations for pharmacological administration are also provided. Vaccine formulations can be, for example, DNA vaccine formulations, recombinant vaccine formulations, or T cell epitope vaccine formulations.

Use of isolated and purified antibodies to Leptospira LP1454, LP1118, LP1939, MCEII, CADF-like1, CADF-like2, CADF-like3, Lp0022, Lp1499, Lp4337, Lp328 or L21 OMPS to improve vaccines for mammals, including but not limited to cow, pig, dog, sheep and horse, is also provided.

Vaccines and methods for making the vaccines that protect a mammal against a Leptospira-related disorder are provided. In particular, a vaccine is provided that elicits active immunity against a leptospirosis infection that contains at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21.

A DNA vaccine is provided that elicits active immunity against a leptospirosis infection comprising a DNA encoding at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21.

A vaccine for providing passive immunity to a leptospirosis infection is also provided. The vaccine can comprise antibodies that are directed against at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21. The antibodies can be selected from the group consisting of polyclonal antibodies and monoclonal antibodies against at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21. In a preferred embodiment of the vaccine, the vaccine is provided in a pharmaceutically accepted carrier.

Further, a vaccine for active immunization of a mammal against a leptospirosis infection is provided. The vaccine can comprise an antigen containing at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21. In one embodiment, the antigen is a recombinant polypeptide produced in a plasmid in a microorganism other than Leptospira, preferably, in an E. coli. In a preferred embodiment, the vaccine is provided in a pharmaceutically accepted carrier. In another embodiment, the Leptospira OMP antigen can be provided as a fusion polypeptide wherein an amino end and/or a carboxyl end of the antigen is fused to all or a portion of a polypeptide that facilitates the isolation of the antigen from the microorganism in which the antigen is produced. In a preferred embodiment, the polypeptide is selected from the group consisting of glutathione S-transferase, protein A, maltose binding protein, and polyhistidine.

A vaccine for protecting a mammal from leptospirosis infection is also provided. The vaccine can comprise a DNA that encodes at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21. In a preferred embodiment, the DNA is operably linked to a promoter to enable transcription of the DNA in a mammalian cell. Preferably, the vaccine is provided in a pharmaceutically accepted carrier.

Methods are provided for using Leptospira LP1454, LP1118, LP1939, MCEII, CADF-like1, CADF-like2, CADF-like3, Lp0022, Lp1499, Lp4337, Lp328 or L21 OMPs as vaccines (or to improve vaccines) or as serological diagnostic antigens.

Methods are provided for producing DNA vaccines, recombinant vaccines, and T cell epitope vaccines based on LP1454, LP1118, LP1939, MCEII, CADF-like1, CADF-like2, CADF-like3, Lp0022, Lp1499, LP4337, Lp328 or L21 OMPs.

A method for vaccinating mammal against a leptospirosis infection is also provided. In one embodiment, the method can comprise: (a) providing a recombinant antigen of the Leptospira OMP, wherein the OMP is selected from the group consisting of LP1454, LP1118, LP1939, MCEII, CADF-like1, CADF-like2, CADF-like3, Lp0022, Lp1499, Lp4337, Lp328 and L21 and wherein the OMP is produced from a microorganism culture wherein the microorganism contains a DINA that encodes an antigen of the Leptospira OMP; and (b) vaccinating the mammal. Preferably, the vaccine is in a pharmaceutically accepted carrier.

In a preferred embodiment of the method, the recombinant antigen is a fusion polypeptide that is fused at the amino terminus and/or carboxyl terminus to a polypeptide that facilitates the isolation of the recombinant antigen. In particular, the polypeptide can be all or a portion of the polypeptide selected from the group consisting of glutathione S-transferase, protein A, maltose binding protein, and polyhistidine. Further, the method can comprise producing the antigen from a DNA that is in a plasmid in a microorganism wherein the DNA is operably linked to a promoter which enables transcription of the DNA to produce the recombinant antigen for the vaccine.

A method for vaccinating a mammal against a leptospirosis infection is also provided wherein the method comprises: (a) providing in a carrier solution a DNA in a plasmid that encodes at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21; and (b) vaccinating the mammal with the DNA in the carrier solution. Preferably, the DNA is in a carrier solution that is pharmaceutically accepted for DNA vaccines. In a preferred embodiment, the DNA is operably linked to a promoter to enable transcription of the DNA in a mammalian cell.

A method is further provided for providing passive immunity to a leptospirosis infection in a mammal comprising: (a) providing antibodies selected from the group consisting of polyclonal antibodies and monoclonal antibodies that are directed against at least one epitope of a Leptospira OMP, wherein the Leptospira OMP is selected from the group consisting of LP1454, LP1118, LP1939. MCEII, CADF-like1, CADF-like2, CADF-like3, Lp0022, Lp1499. Lp4337, Lp328 and L21; and (b) inoculating the mammal. Preferably, the antibodies are provided in a pharmaceutically accepted carrier.

Further still, a method for producing an antigen is provided wherein the method comprises: (a) providing a microorganism in a culture containing a DNA encoding a fusion polypeptide comprising at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21, and a polypeptide that facilitates isolation of the fusion polypeptide; (b) culturing the microorganism in a culture to produce the fusion polypeptide; and (c) isolating the fusion polypeptide.

In one embodiment, the fusion polypeptide is isolated by affinity chromatography which can be affinity chromatography that comprises an IgG-linked resin when the polypeptide consists of all or a portion of protein A, a Ni² resin when the polypeptide is polyhistidine, amylose resin when the polypeptide is all or part of the maltose binding protein, or glutathione Sepharose 4B resin when the polypeptide is all or part of glutathione S-transferase.

A vaccine for a mammal is also provided that comprises an isolated recombinant protein encoded by a cDNA produced from RNA of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21 and a vaccine carrier. In another embodiment, the vaccine for a mammal comprises a recombinant virus vector containing DNA encoding an epitope of a Leptospira OMP, wherein the Leptospira OMP is selected from the group consisting of LP1454, LP1118, LP1939, MCEII, CADF-like1, CADF-like2, CADF-like3, Lp0022, Lp499, Lp4337, Lp328 and L21, and a vaccine carrier. In another embodiment, the present invention comprises a DNA vaccine for a mammal comprising a plasmid containing DNA encoding an epitope of a Leptospira OMP.

A method for protecting a mammal against a leptospirosis infection is also provided that comprises providing a vaccine that when injected into the mammal causes the mammal to produce antibodies and cell mediated immunity against a Leptospira OMP antigen wherein the antibodies prevent infection by the Leptospira organism. In particular, the vaccine comprises at least one epitope of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21 in a vaccine carrier. A vaccine is further provided comprising a recombinant virus vector that expresses the Leptospira OMP antigen. The present invention further still provides a vaccine which comprises a DNA plasmid encoding the Leptospira OMP antigen.

The present invention further comprises a monoclonal antibody that selectively binds to at least one epitope of a Leptospira OMP, wherein the Leptospira OMP is selected from the group consisting of LP1454, LP1118, LP1939 MCEII, CADF-like1, CADF-like2, CADF-like3, Lp0022, Lp1499, Lp4337, Lp328 and L21.

Kits are provided comprising in one or more containers an antibody of the invention that immunospecifically binds to a Leptospira LP1454, LP1118, LP1939, MCEII, CADF-like1, CADF-like2, CADF-like3, Lp0022, Lp1499, Lp4337, Lp328 or L21 protein.

Kits are also is provided comprising in one or more containers a vaccine of the invention.

Kits are also is provided comprising in one or more containers a plasmid comprising a LP1454, LP1118, LP1939, MCEII, CADF-like1, CADF-like2, CADF-like3, Lp0022, Lp1499, Lp4337, Lp328 or L21 gene in an expression vector.

5.8.1 DNA Vaccine

A recombinant DNA vaccine is provided. In one embodiment, the DNA vaccine comprises:

a) a recombinant DNA wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above; and
   b) a vector capable of expressing the recombinant DNA when the recombinant DNA is inserted into the vector, wherein the recombinant DNA is inserted into the vector such that a recombinant protein is expressed when the vector is provided in an appropriate host.

A DNA vaccine can be constructed by subcloning the gene of interest into a eukaryotic plasmid vector. Candidate vectors can include, but are not limited to, pcDNA3, pCI, VR1012, and VR1020, all of which are vectors well known in the art. This construct can then be used as a vaccine.

pcDNA3 is well known in the art as a cloning vector and is commercially available from e.g., Invitrogen, Carlsbad, Calif. pCI is well known in the art as a mammalian expression vector and is commercially available from e.g., Promega Corporation, Madison, Wis., Vectors VR1012 and VR1020 are both known in the art and available from Vical, San Diego, Calif.

A LP1454, LP1118, LP1939, MCEII, CADF-like1, CADF-like2, CADF-like3, Lp0022, Lp1499, Lp4337, Lp328 or L21 protein can be used to create a DNA vaccine (reviewed in Robinson, 1997). In addition, any immunologically active portion of these proteins is a potential candidate for the vaccine. A plasmid containing one of these genes in an expression vector is constructed. The gene must be inserted in the correct orientation in order for the genes to be expressed under the control of eukaryotic promoters. Possible promoters include, but are not limited to, the cytomegalovirus (CMV) immediate early promoter, the human tissue plasminogen activator (t-PA) gene (see, e.g., Degen et al., 1986, J. Biol. Chem. 1986 May 25; 261(15):6972-85), and the promoter/enhancer region of the human elongation factor alpha (EF-1α) (see, e.g., Kim et al., Use of the human elongation factor 1 alpha promoter as a versatile and efficient expression system, Gene. 1990 Jul. 16; 91 (2):217-23; Uetsuki et al., Isolation and characterization of the human chromosomal gene for polypeptide chain elongation factor-1 alpha. J. Biol. Chem. 1989 Apr. 5; 264(10):5791-8). Orientation can be identified by restriction endonuclease digestion and DNA sequencing.

Expression of these gene products can be confirmed by indirect immunofluorescent staining of transiently transfected COS cells, CHO cells, or other suitable cells. The same plasmid without these genes is used as a control. Plasmid DNA is transformed into E. coli DH5α. DNA can be purified using art-known methods, e.g., by cesium chloride gradients, and the concentration can be determined by a standard protocol known in the art (Nyika et al., 1998, A DNA vaccine protects mice against the rickettsial agent Cowdria ruminantium, Parasite Immunol. 1998 March; 20(3):111-9).

Once the DNA is purified, the vector containing the insert DNA can be suspended in phosphate buffer saline solution and directly injected into test animals such as a dog or cattle. Inoculation into a muscle can be done using standard methods, with a needle or intravenously. Alternatively, a gene gun can be used to transport DNA-coated gold beads into cells using standard art-known methods (Fynan et al., 1993, DNA vaccines: protective immunizations by parenteral, mucosal, and gene-gun inoculations, Proc. Nat'l Acad. Sci. U.S.A. 1993 Dec. 15; 90(24):11.478-82.).

The inoculated host can express the plasmid DNA in its cells, and can produce a protein that raises an immune response. Each of the newly identified genes can be used to create a vaccine by this technique.

CpG molecules can be used as an adjuvant in the vaccine (Klinman et al., 1997, Contribution of CpG motifs to the immunogenicity of DNA vaccines. J. Immunol. 1997 Apr. 15; 158(8):3635-9). Adjuvants are materials that help antigens or increase the immune response to an antigen. The motifs consist of an unmethylated CpG dinucleotide flanked by two 5′ purines and two 3′ pyrimidines. Oligonucleotides containing CpG motifs have been shown to activate the immune system, thereby boosting an antigen-specific immune response. This effect can be utilized by mixing the CpG oligonucleotides with the DNA vaccine, or physically linking the CpG motifs to the plasmid DNA using standard techniques known in the art.

In a specific embodiment, a method for producing a vaccine against a Leptospira-related disorder is provided comprising: a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
   b) providing a vector capable of expressing the recombinant DNA when the recombinant DNA is inserted into the vector and
   c) inserting the recombinant DNA into the vector, wherein the recombinant DNA is inserted into the vector such that a recombinant protein is expressed when the vector is provided in an appropriate host.

In another embodiment, a method for producing a vaccine against a Leptospira-related disorder is provided comprising:

a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ II NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (LP328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
   b) constructing an expression vector comprising a plasmid, wherein the plasmid comprises the recombinant DNA; and
   c) inoculating a host with the expression vector, wherein the recombinant DNA is expressed in the host to produce a recombinant protein, and wherein the expressed protein raises an immune response in the host.

5.8.2 Recombinant Vaccine

A recombinant vaccine is provided. In one embodiment, the vaccine comprises: a recombinant protein, wherein the recombinant protein is selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 90% or 95% of the length of the amino acid sequence of any of the above.

To develop a recombinant vaccine, an OMP gene, such as LP1454, LP1118, LP1939, MCEII, CADF-like1, CADF-like2, CADF-like3, Lp0022, Lp1499, Lp4337, Lp328 or L21, is individually subcloned into overexpression vectors, and then purified for vaccine development using methods known in the art. The OMP of interest can be expressed in a plasmid with a strong promoter such as the tac, T5, or T7 promoter. Alternatively, immunologically active fragments of the OMP can be used in the development of a vaccine. Each of these genes can be subcloned into a plasmid and transformed into an E. coli strain as described above.

The recombinant protein can be overexpressed, using art-known techniques, in a vector with a strong promoter. Vectors that can be suitable for use in this technique include pREST (Invitrogen Inc., Calif.), pKK233-3 (Pharmacia, Calif), and the pET system (Promega. Wis.), although any vector with a strong promoter can be used. After overexpression, the proteins can be purified and mixed with adjuvant. Potential adjuvants include, but are not limited to, aluminum hydroxide, Quil A or MONTANIDE®, Quil A is well known in the art as a highly refined form of saponin, a plant-derived biochemical that stimulates both cell-mediated and humoral immune responses. MONTANIDE®” is U.S. registered trademark for a specific formulation for an emulsifier produced by the Seppic company (Paris, France). MONTANIDE® is well known in the art for its use as an adjuvant.

The purified protein can be used as immunogen to vaccinate dogs by techniques known in the art (see, e.g., Palaniappan R U, et al. Immunoprotection of recombinant leptospiral immunoglobulin-like protein A against Leptospira interrogans serovar Pomona infection. Infect Immun. 2006 March; 74(3): 1745-50, incorporated herein by reference).

Briefly, the individual protein is expressed and purified from E. coli. Then, test animals, such as dogs or cattle, are injected intramuscularly or subcutaneously with the purified recombinant vaccine and adjuvant. This injection can elicit an immune response.

In one embodiment, a method for producing a vaccine against a Leptospira-related disorder comprises:

a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022)
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
   b) providing a vector capable of expressing the recombinant DNA when the recombinant DNA is inserted into the vector;
   c) inserting the recombinant DNA into the vector;
   d) providing a bacterial strain;
   e) transforming the vector into the bacterial strain such that a recombinant protein is expressed when the vector is transformed into the bacterial strain; and
   f) harvesting the recombinant protein from the bacterial strain.

In another embodiment, a method for producing a recombinant vaccine is provided comprising:

subcloning a LP1454, LP1118, LP1939, MCEII, CADF-like1, CADF-like2, CADF-like3, Lp0022, Lp1499, Lp4337, Lp328 or L21 gene or a DNA sequence encoding an immunologically active portion of a LP1454, LP1118, LP1939, MCEII, CADF-like1, CADF-like2, CADF-like3, Lp0022, Lp1499, Lp4337, LP328 or L21 protein into an overexpression vector,

expressing a LP1454, LP1118, LP1939, MCEII, CADF-like1, CADF-like2, CADF-like3, Lp0022, Lp1499, Lp4337, Lp328 or L21 protein, peptide or immunologically active fragment; and

purifying the expressed LP1454, LP1118, LP1939, MCEII, CADF-like1, CADF-like2, CADF-like3, Lp0022, Lp1499, Lp4337, Lp328 or L21 protein, peptide or immunologically active fragment.

5.8.3 Epitope Mapping and T-Cell Epitope Vaccine

Direct cell cytotoxicity mediated by CD8⁺ T lymphocytes (CTL) is the major mechanism of defense against intracellular pathogens. These effector lymphocytes eliminate infected cells by recognizing short peptides associated with MHC class I molecules on the cell surface. Exogenous antigens enter the endosomal pathway and are presented to CD4⁺ T cells in association with class II molecules whereas endogenously synthesized antigens are presented to CD8⁺ T cells in association with MHC class I molecules.

Leptospira is an intracellular pathogen that resides in monocytes and macrophages. A Leptospira-specific CTL response can be generated that can eliminate the organism from monocytes or macrophages of infected animals.

To increase the protective response of a protein vaccine, a test animal can be immunized with selective epitopes of the protein. The rationale behind this is that an epitope vaccine contains the most relevant immunogenic peptide components without the irrelevant portions. Therefore, a search can be performed for the most highly antigenic portions of the newly identified proteins.

In one embodiment, a T-cell epitope vaccine is provided. The T-cell epitope vaccine can comprise a recombinant protein, wherein the recombinant protein comprises a T-cell epitope, and wherein the T-cell epitope comprises an amino acid peptide fragment of a protein selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939).
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and a protein fragment of at least 10%, 20%, 30%, 40%, 50%, 60% 70%, 80%, 90% or 95% of the length of the amino acid sequence of any of the above.

To identify) T-cell epitopes from a newly discovered. OMP or putative OMP, an initial electronic search for homologous sequences to known T-cell epitopes is performed using standard art-known searching methods such as BLAST searching in the NCBI databases.

In one embodiment, a T-cell epitope consisting of or comprising a fragment of a Leptospira OMP, wherein the Leptospira OMP is selected from the group consisting of LP1454, LP1118, LP1939, MCEII, CADF-like1, CADF-like2, CADF-like3, Lp0022, Lp1499, Lp4337, Lp328 and L21 is provided. In a specific embodiment, the T-cell epitope consists of at least 10 (continuous) amino acids of the Leptospira OMP is provided. In other embodiments, the fragment consists of at least 20 or 50 amino acids of the Leptospira OMP protein. In specific embodiments, such fragments are not larger than 35, 100 or 200 amino acids. Derivatives or analogs of Leptospira OMP T-cell epitopes include but are not limited to those molecules comprising regions that are substantially homologous to Leptospira OMP T-cell epitopes or fragments thereof (e.g., in various embodiments, at least 60% or 70% or 80% or 90% or 95% identity over an amino acid sequence of identical size or when compared to an aligned sequence in which the alignment is done by a computer homology program known in the art) or whose encoding nucleic acid is capable of hybridizing to a coding Leptospira OMP epitope sequence, under stringent, moderately stringent, or nonstringent conditions.

In addition, extensive T-cell epitope mapping can be carried out. Each LP1454, LP1118, LP1939, MCEII, CADF-like1, CADF-like2, CADF-like3, Lp0022, Lp1499, Lp4337, Lp328 or L21 OMP can be tested for immunogenic peptide fragments. Mapping of T-cell epitopes is well known in the art (see, e.g., Launois et al., 1994, T-cell-epitope mapping of the major secreted mycobacterial antigen Ag85A in tuberculosis and leprosy, Infect Immun. 1994 September; 62(9):3679-87; Lee and Horwitz, 1999, T-cell epitope mapping of the three most abundant extracellular proteins of Mycobacterium tuberculosis in outbred guinea pigs, Infect Immun. 1999 May; 67(5):2665-70).

Briefly, short, overlapping peptide sequences (9-20 amino acids) can be synthesized over the entire length of the protein in question. These short peptide fragments can be tested using healthy cows, which can be immunized with the protein of interest. Peripheral blood mononuclear cells from the cows can be tested for T-cell stimulatory and IFN-γ inducing properties. Those fragments that elicit the strongest response may be preferred candidates for a T-cell epitope vaccine.

Once fragments are identified that can make a preferred epitope, a recombinant adenylate cyclase of Bordetella bronchiseptica is constructed carrying a Leptospira CD8⁺ T cell epitope. The adenylate cyclase toxin (CyaA) of Bordetella bronchiseptica elicits an immune response. In addition, CyaA is well suited for intracytoplasmic targeting. Its catalytic domain (AC), corresponding to the N-terminal 400 amino acid residues of the 1,706-residue-long protein, can be delivered to many eukaryotic cells, including cells of the immune system. Also, toxin internalization is independent of receptor-mediated endocytosis, suggesting that the catalytic domain can be delivered directly to the cytosol of target cells through the cytoplasmic membrane.

The Pseudomonas aeruginosa exotoxin A (PE) is another toxin that can be used in this procedure to deliver peptides or proteins into cells (Donnelly et al., 1993, Targeted delivery of peptide epitopes to class I major histocompatibility molecules by a modified Pseudomonas exotoxin. Proc Natl Acad Sci USA. 1993 Apr. 15; 90(8):3530-4).

Foreign peptides (16 residues) have been inserted into various sites of the AC domain of CyaA without altering its stability or catalytic and calmodulin-binding properties. Thus, protein engineering allows the design and delivery of antigens that specifically stimulate CTLs.

The adenylate cyclase (AC) toxin (cya) gene of B. bronchiseptica has been cloned. A synthetic double-stranded oligonucleotide encoding a 9 to 20 amino acid class I T cell epitope of either LP1454, LP1118, LP1939 or MCEII, can be designed using standard methods according to B. bronchiseptica codon usage. The complementary oligonucleotides can be inserted in the hypervariable region of the cloned AC-coding sequence of the cya. This technique is known in the art in other systems (see, e.g., Sebo et al., 1995, Cell-invasive activity of epitope-tagged adenylate cyclase of Bordetella pertussis allows in vitro presentation of a foreign epitope to CD8+ cytotoxic T cells, Infect Immun. 1995 October; 63(10):3851-7; Guermonprez et al., 1999, Direct delivery of the Bordetella pertussis adenylate cyclase toxin to the MHC class I antigen presentation pathway. J. Immunol. 1999 Feb. 15; 162(4): 1910-6).

Recombinant plasmids carrying the chimeric cya gene can be sequenced using standard sequencing methods to determine the copy number and orientation of the inserted epitope. A plasmid with a complete copy of the insert that specifies the T-cell epitope (CD8⁺) in the correct orientation can be chosen from the sequenced plasmids. The ability of the new chimeric protein to enter eukaryotic cells can be used to ensure intracellular targeting of the epitopes (Fayolle et al., 1996, Therapy of murine tumors with recombinant Bordetella pertussis adenylate cyclase carrying a cytotoxic T cell epitope, J. Immunol. 1999 Apr. 1; 162(7):4157-62).

A vaccine can be created in several ways. For example, a recombinant chimeric protein can be purified and used to inoculate test animals, e.g., dogs or cows. Alternatively, an attenuated B. bronchiseptica strain that carries a T-cell epitope gene or a LP1454, LP1118, LP1939, MCEII, CADF-like1, CADF-like2, CADF-like3, Lp0022, Lp1499, Lp4337, Lp328 or L21 gene by in-frame insertion into adenylate cyclase can be created by allelic-exchange as known in the art (Cotter P A and Miller J F. BvgAS-mediated signal transduction: analysis of phase-locked regulatory mutants of Bordetella bronchiseptica in a rabbit model. Infect Immun. 1994 August; 62(8):3381-90).

In a specific embodiment, a method for producing a T-cell epitope vaccine is provided that comprises:

a) providing a recombinant protein that comprises a T-cell epitope, wherein the T-cell epitope comprises an amino acid peptide fragment of a protein selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and a protein fragment of at least 10%, 20%, 30%, 40%, 50%, 60% 70%, 80%, 90% or 95% of the length of the amino acid sequence of any of the above;
   b) identifying the T-cell epitope from the protein;
   c) inserting DNA encoding the T-cell epitope into a construct capable of expressing the T-cell epitope as a protein; and
   d) harvesting the protein.

Finally, protection against leptospirosis infection in test animals, e.g., dogs, cows, pigs, or horses, that are vaccinated with the adenylate cyclase-LP1454, -LP1118, -LP1939, -MCEII, -CADF-like1, -CADF-like2, -CADF-like3, -Lp0022, -Lp1499, -Lp4337, -Lp328 or -L21 chimeric protein can be determined. Using standard methods, wild type and recombinant ACs and CyAs can be diluted to working concentrations in PBS and the chimeric protein can be injected into test animals, e.g., dogs or cows, either intramuscularly or subcutaneously. Alternatively, the T-cell epitope can be inserted into the adenylate cyclase gene of an attenuated B. bronchiseptica strain in frame, and the test animals subsequently vaccinated or inoculated with live bacteria.

Recombinant antigens are promising candidates for human and animal vaccination against various pathogens. However, a serious drawback is the poor immunogenicity of recombinant antigens as compared to native antigens. A major challenge in the development of a new recombinant vaccine is, therefore, to have a new adjuvant system that increases the immunogenicity of antigens. Cytokines are powerful immunoregulatory molecules. Cytokines that can be used as adjuvants include, but are not limited to, IL-12 (interleukin-12), GM-CSF (granulocyte-macrophage colony stimulating factor), IL-1β (interleukin-1β) and γ-IFN (gamma interferon).

These cytokines can have negative side effects including pyrogenic and/or proinflammatory symptoms in the vaccinated host. Therefore, to avoid the side effects of a whole cytokine protein, an alternate approach can be to use synthetic peptide fragments with the desired immunostimulatory properties. The nonapeptide sequence VQGEESNDK (SEQ ID NO: 15) of IL-1β protein is endowed with powerful immuno-enhancing properties, and is discussed here to illustrate the use of a cytokine to increase immunogenicity.

This nonapeptide can be inserted into the OMP, e.g., a LP1454, LP1118, LP1939 or MCEII protein, and its immunogenicity can be compared to that of the native protein. Reportedly, the insertion of this sequence into a poorly immunogenic recombinant antigen increases the chance of a strong protective immune response after vaccination. This peptide can enhance the in vivo immune response against both T-dependent and T-independent antigens. The bovine IL-1β, sequence may mimic many immunomodulatory activities of the entire molecule of IL-1β while apparently lacking many of its undesirable proinflammatory properties. This strategy can be employed to increase the immunogenicity of the OMP antigens.

The efficacy of the recombinant proteins as vaccines can be tested in cattle, pigs, horses or dogs. The purified protein can be injected, e.g., intraperitoneally, into the animal of interest.

For example, when testing the efficacy of the recombinant protein as vaccines in dogs, specific pathogen free (SPF) dogs can be used. The SPF dogs can be divided into five groups: one group can be given recombinant adenylate cyclase of Bordetella bronchiseptica carrying CD8⁺ T cell epitopes derived from LP1454, LP1118, LP1939, MCEII, CADF-like1, CADF-like2, CADF-like3, Lp0022, Lp1499, Lp4337, Lp328 or L21, one group is given recombinant adenylate cyclase of Bordetella bronchiseptica as a control, one group can be given the LP1454, LP1118, L1939, MCEII, CADF-like1, CADF-like2, CADF-like3, Lp0022, Lp1499, Lp4337, Lp328 or L21 protein plus a bovine IL-1β 163-171 insert, one group can be given a T cell epitope derived from LP1454, LP1118, LP1939, MCEII, CADF-like1, CADF-like2, CADF-like3, Lp0022, Lp1499, Lp4337, Lp328 or L21 alone, and the last group can be given PBS as a negative control.

Animals can be vaccinated using standard methods known in the art (see, e.g., Chang. Y.-F. et al. 1995. Recombinant OspA Protects Dogs against Infection and Disease Caused by Borrelia burgdorferi, Infection and Immunity 63 (9): 3543-3549).

For example, dogs can be vaccinated twice (e.g., 30-40 μg for each vaccination). Dogs can be challenged 21 days after the last vaccination with a virulent strain of Leptospira spp. At day five postchallenge, approximately 1 ml blood from each dog can be collected in an EDTA tube. Whether the vaccinated groups eliminate the organisms as compared to that of the control group can be tested by standard culture and PCR methods.

Amplification of the gene product can be performed using standard methods known in the art. For example, two primers derived from the genes cloned can be used to amplify the gene product from the tissues or blood samples from these dogs. The internal primer can also be designed for use as an oligonucleotide probe to hybridize the PCR gene product.

### 5.9 Methods for Prevention and for Treatment of a Leptospira-Related Disorder

A method is provided for preventing a Leptospira-related disorder in an animal or human subject in need thereof, comprising administering an amount of antibody of the invention sufficient to confer immunity to the Leptospira-related disorder to the subject.

A method is also provided for preventing a Leptospira-related disorder in an animal or human subject in need thereof, comprising administering an amount of a vaccine of the invention sufficient to confer immunity to the Leptospira-related disorder to the subject.

A method is also provided for treating Leptospira-related disorder in an animal or human subject in need thereof, comprising administering an amount of an antibody of the invention sufficient to inhibit or decrease the activity of a Leptospira pathogen.

In one embodiment, the method comprises administering an amount of a vaccine of the invention sufficient to inhibit or decrease the activity of the Leptospira pathogen.

In another embodiment, the amount of the vaccine of the invention administered is sufficient to confer immunity to Leptospira infection or a Leptospira-related disorder to the subject.

In another embodiment, the method for treating the Leptospira-related disorder may not involve administration of antibodies or a vaccine of the invention to a subject. For example, antibodies of the invention can be used to kill infectious organisms in vitro where eventual intended use is to combat infection in animals or humans.

### 5.10 Methods for Assaying, Diagnosis and Monitoring Therapy

Methods are provided for assaying for the presence or activity of a LP1454, LP1118, LP1939, MCEII, CADF-like1, CADF-like2, CADF-like3, Lp0022, Lp1499, Lp4337, Lp328 or L21 protein using antibodies to Leptospira LP1454, LP1118, LP1939, MCEII, CADF-like1, CADF-like2, CADF-like3, Lp0022, Lp1499, Lp4337, Lp328 or L21 OMPS or fragments thereof:

Methods are provided for diagnosing a Leptospira-related disorder in a subject comprising:

providing one or more antibodies to a Leptospira LP1454, LP1118, LP1939, MCEII, CADF-like1, CADF-like2, CADF-like3, Lp0022, Lp1499, Lp4337, Lp328 or L21 OMP or fragment thereof;

collecting a cell or tissue sample, e.g., a blood sample, from the subject,

contacting the sample with the antibodies; and

assaying for the presence or activity of a LP1454, LP1118, LP1939, MCEII, CADF-like1, CADF-like2, CADF-like3, Lp0022, Lp1499, Lp4337, Lp328 or L21 protein in the sample using antibodies to the OMP or fragment thereof;

wherein presence or activity of a LP1454, LP1118, LP1939, MCEII, CADF-like1, CADF-like2, CADF-like3, Lp0022, Lp1499, Lp4337, Lp328 or L21 protein is indicative of the presence of the Leptospira-related disorder in the subject.

Methods are provided for diagnosing or screening for the presence of a Leptospira-related disorder in a subject comprising measuring the level of a LP1454, LP1118, LP1939, MCEII, CADF-like1, CADF-like2, CADF-like3, Lp0022, Lp1499, Lp4337, Lp328 or L21 protein or protein functional activity in a sample derived from the subject, in which a decrease in the level of LP1454, LP1118, LP1939, MCEII, CADF-like1, CADF-like2, CADF-like3, Lp0022, Lp1499, Lp4337, Lp328 or L21 protein or protein functional activity in the sample, relative to the level of LP1454, LP1118, LP1939, MCEII, CADF-like1, CADF-like2, CADF-like3, Lp0022, Lp1499, Lp4337, Lp328 or L21 protein or protein functional activity found in an analogous sample not having the disorder, indicates the presence of the disease or disorder or a predisposition for developing the disorder.

Methods are also provided for monitoring therapy using antibodies to Leptospira LP454, LP1118, LP1939, MCEII, CADF-like1, CADF-like2, CADF-like3, Lp0022, Lp1499, Lp4337, Lp328 or L21 OMPs or fragments thereof.

Methods for using recombinant LP1454, LP1118, LP1939, MCE, CADF-like1, CADF-like2, CADF-like3, Lp0022, Lp1499, Lp4337, Lp328 or L21 proteins as diagnostic agents are also provided. In one embodiment, a LP1454, LP1118, LP1939, MCEII, CADF-like1, CADF-like2, CADF-like3, Lp0022, Lp1499, Lp4337, LP328 or L21 protein is used in a kinetic enzyme-linked immunosorbent assay (KELA ELISA).

The following examples are offered by way of illustration and not by way of limitation.

### 6. EXAMPLES

### 6.1 Example 1

### Identification of Immunogenic Proteins from Genome-Derived Putative Outer Membrane of Leptospira

6.1.1 Introduction

Study of Leptospira outer membrane proteins (OMPs) can help in the understanding Leptospira mode of infection, since Leptospira utilize membrane proteins extensively during infection. Since leptospires survive outside as well as inside the host, some OMPs are differentially regulated, for examples lipoprotein LipL36 is downregulated whereas Lig proteins are upregulated during infection (Matsunaga J., M. A. Barocchi, J. Croda, T. A. Young, Y. Sanchez, I. Siqueira, C. A. Bolin, M. G. Reis, L. W. Riley, D. A. Haake, and A. I. Ko. 2003. Pathogenic Leptospira species express surface-exposed proteins belonging to the bacterial immunoglobulin superfarmily Mol. Microbiol. 49:929-945, Palaniappan, R. U., Y. F. Chang, S. S. Jusuf. S. Artiushin, J. F. Timoney, S. P. McDonough, S. C. Barr, T. J. Divers, K. W. Simpson, P. L. McDonough, and H. O. Mohammed. 2002. Cloning and molecular characterization of an immunogenic LigA protein of Leptospira interrogans, Infect. Immun. 70:5924-5930).

Therefore, the identification of outer membrane proteins that are expressed only during infection can help to develop diagnostic reagents for leptospirosis. 15 putative outer membrane proteins of Leptospira that did not show any significant homology with other organisms in the NCBI database were randomly selected from the available genomic sequences of Leptospira and expressed as GST fusion proteins in Escherichia coli. Purified recombinant putative outer membrane proteins, along with recombinant conserved region of LigA and B, variable region of LigB and LipL32, were evaluated by immunoblot analysis with bovine sera from naturally infected, experimentally infected and healthy animals. Except for recombinant (“r”) protein, rLA1947, all the recombinant outer membrane proteins (OMPs) showed reactivity with experimentally infected bovine sera. However, variable reactivity of OMPs was found with naturally infected sera, Nevertheless, rLP1939, and rMCEII showed consistently strong reactivity to all the naturally infected animals irrespective of MAT titer indicating that these immunogenic proteins of Leptospira can serve as effective diagnostic tools or vaccine for leptospirosis.

We investigated the expression and immunogenicity of genome-derived putative outer membrane proteins for the development of effective diagnostic reagents and/or recombinant proteins/DNA vaccine for leptospirosis. We selected 15 outer membrane proteins that did not show any significant homology to other proteins in the NCBI database and expressed them as GST fusion proteins in E. coli. Antigenic analysis of the purified recombinant OMPs, along with conserved region of LigA and B and variable region of LigB and LipL32, were evaluated by immunoblot with sera from experimentally and naturally infected cattle. The data indicate that rLP1939 and rMceII show consistently stronger reactivity with infected serum samples indicating these proteins can serve as effective diagnostic antigens for leptospirosis.

6.1.2 Materials and Methods.

Bacterial strains and growth. L. interrogans serovar Pomona (NVSL 1427-35-093002) was used in this study. Leptospires were grown on EMJH medium at 30° C. Growth was monitored by dark-field microscopy.

Antisera. Experimentally infected bovine sera to serovars Cannicola, Copenhageni, Grippotyphosa and Hardjobovis were previously described (Surujballi, O. P., R. M. Marenger. M. D. Eaglesome, and E. A. Sugden. 1997. Development and initial evaluation of an indirect enzyme-linked immunosorbent assay for the detection of Leptospira interrogans serovar hardjo antibodies in bovine sera. Can. J. Vet. Res. 61:260-266). Infected animals were identified by exhibition of clinical symptoms and MAT. Naturally infected bovine sera were obtained from the New York State Animal Health Diagnostic Center at Cornell University (Ithaca, N.Y.) from animals that tested positive for leptospiral infection by MAT. Negative control bovine sera were obtained from healthy animals using standard methods known in the art. All the sera were collected from the jugular vein and stored at −20° C. until use.

Microscopic Agglutination Test (MAT). MAT was carried out as previously described (Cole, J. R., Jr., H. C. Ellinghausen, and H. L. Rubin. 1979. Laboratory diagnosis of leptospirosis of domestic animals. Proc. Annu. Meet. U S Anim. Health Assoc: 189-195) with the whole cell antigens of the following serovars: Pomona, Grippotyphosa, Icterohaemorrhagiae, Hardjo, Canicola, Autumnalis and Bratislava.

Cloning and expression of leptospiral OMPs. Primers for the putative outer membrane protein genes were designed without the signal sequences using standard methods known in the art and are listed in Table 1.

TABLE 1
Primers used to amplify and clone L. interrogans serovar
specific sequences

| | | | | Calculated | |
|---|---|---|---|---|---|
| | GenBank | SEQ. | | mol | Base |
| Gene | Accession | ID | | Weight | pair |
| no.^{a} | Number | NO: | Primer sequences^{c} | (kDa) | (bp) |
| LP1118 | AAN48316 | 16 | CGCGGATCCACGTTAGGCAACT | 34.8 | 873 |
| | | 17 | CCGCTCGAGTTAGTTTTGTTT | | |
| LP1228 | AAN48427 | 18 | CGCGGATCCAATCCGATAACGA | 23 | 507 |
| | | | AT | | |
| | | 19 | CCGCTCGAGTTAATGATATTCGT | | |
| | | | TTT | | |
| LP1404 | AAN48603 | 20 | CGCGGATCCAGAGTTCCTTTTTT | 43 | ~1100 |
| | | | T | | |
| | | 21 | CCGCTCGAGTTAAGGAACGTTT | | |
| MCEI | AAN49254 | 22 | TTTGGATCCGCGATTACCGTA | 27 | 753 |
| LP2025 | | 23 | CTTCTCGAGTTAAAAGCACTT | | |
| LP2268 | AAN49267 | 24 | CGCGGATCCCCCGACTTAAATG | 50 | 1056 |
| | | | T | | |
| | | 25 | CCGCTCGAGTCAAAGTATCGAA | | |
| | | | T | | |
| LigBVT | AF534640 | 26 | CGCGGATCCGTCAATAACAACA | 66 | 1500 |
| | | | TTGC | | |
| | | 27 | CCGCTCGAGTTGGTTTCCTTTTA | | |
| | | | CGTT | | |
| LP1332 | AAN48531 | 28 | CGCGGATCCATGTTGTTACAC | 36 | 984 |
| | | 29 | CCGCTCGAGCTACTGAGAAATC | | |
| | | | TTT | | |
| LP1965 | AAN49164 | 30 | CGCGGATCCTCTCCCGAATGG | 28 | ~710 |
| | | 31 | CCGCTCGAGTTATCTCGTATCAA | | |
| | | | A | | |
| LP1192 | AAN48391 | 32 | CGCGGATCCATAGTACGTTTAA | 59.5 | ~1500 |
| | | | AA | | |
| | | 33 | CCGCTCGAGTTAAAAACTGTGG | | |
| | | | GA | | |
| LP1947 | AAN49146 | 34 | CGCGGATCCCAATCAAAATCGG | 45 | 1140 |
| | | | C | | |
| | | 35 | CCGCTCGAGTTAATCCTCTAAAT | | |
| | | | CT | | |
| LigCon | AF534640 | 36 | TCCCCCGGGGCTGGCAAAAGA | 66 | 1797 |
| | | 37 | CCCTCGAGAATATCCGTATTAG | | |
| | | | A | | |
| LP2637 | AAN49836 | 38 | AGGGGAATTCTATGAAAAACTT | 32 | 900 |
| | | | TCGAT | | |
| LIPL32 | | 39 | CCCTCGAGCTTAGTCGCGTCAG | | |
| LP1495 | AAN48694 | 40 | CGCGGATCCGATCAGCAACT | 37.5 | 921 |
| | | | T | | |
| | | 41 | CCGCTCGAGTTAATTTTGTGTTT | | |
| | | | TT | | |
| LP1939 | AAN49138 | 42 | CGCGGATCCTGCAAACAAGAT | 17 | 411 |
| | | 43 | CGCCTCGAGTTATTGAGAAGCG | | |
| | | | TAT | | |
| MCEII | AAN47806 | 44 | CGCGGATCCGAAAAAGCCGGAA | 25 | 718 |
| LP0607 | | 45 | CCGCTCGAGTTATAACTTTCCGA | | |
| | | | AAT | | |
| LP2471 | AAN49670 | 46 | CGCGGATCCTTTCCAGAAATTTC | 37.5 | 909 |
| | | | T | | |
| | | 47 | CCGCTCGAGTTAATAATATCTTT | | |
| | | | GT | | |
| LP1931 | AAN49130 | 48 | CGCGGATCCATTGCACAAATC | 42 | ~1100 |
| | | 49 | CCGCTCGAGTCAAAGAGCAAAC | | |
| | | | TT | | |
| LP1454 | AAN48653 | 50 | CGCGGATCCGAAGAAAGATCCT | 42 | 981 |
| | | 51 | CCGCTCGAGTCATCTTTTATCTT | | |
| | | | T | | |

^{a}Gene numbers are from GenBank and HTTP://www.chgc.org.cn/.
^{b}For, forward; Rev, reverse.
^{c}Listed 5′ to 3′
^{d}N indicates no confirmed location in the genome of Leptospira

Genomic DNA of L. interrogans serovar Pomona was prepared using a DNAeasy kit (Qiagen, Valencia, Calif.) and subjected to PCR as described by the manufacturer using Accuprime Taq polymerase (Invitrogen, CA). PCR products were run in a 1% agarose gel and visualized by ethidium bromide staining. PCR products were eluted from the gel using a gel elution kit (Qiagen, Valencia, Calif.) and cloned into TOPO TA vector as described by the manufacturer (Invitrogen, CA). Except for LP1947, which was subcloned in pRSETA (InVitroGen, CA), all inserts were sub-cloned into the expression vector pGEX-KG (Stratagene) and transformed into E. coli (BL21 DE3 or CQ2). DNA sequencing was performed with an ABI model 377 automated nucleic acid sequencer at the Bioresource Center, Cornell University. Homology searches were performed using the NCBI database and BLAST (Altschul, S. F., W. Gish, W. Miller, E. W. Myers, and D. J. Lipman. 1990. Basic local alignment search tool. Mol. Biol. 215:403-410).

Expression and purification of recombinant proteins. Exponentially growing cultures (OD₆₀₀=0.6) of E. coli (BL21 DE3) or CQ21 harboring recombinant plasmids were induced to synthesize the fusion proteins with 1 mM isopropyl-β-D-thiogalactopyranoside (IPTG; Sigma. St. Louis, Mo.). For each recombinant protein, a pilot experiment was performed with 3 mL culture volume and the molecular weight of the expressed recombinant protein was checked by SDS-PAGE analysis. Mass cultivation was performed in 500 mL of LB broth with ampicillin (50 μg/mL). Bacteria were harvested by centrifugation at 5,000 RPM for 30 min and the cell pellets were washed and suspended in PBS followed by passing through a French pressure cell (American Instruments). The lysates were centrifuged at 8,000×g for 30 min and the inclusion bodies were washed and purified (Palaniappan, R. U., Y. F. Chang, F. Hassan, S. P. McDonough, M. Pough, S. C. Barr, K. W. Simpson, H. O. Mohammed, S. Shin, P. McDonough, R. L. Zuerner, J. Qu, and B. Roe. 2004. Expression of leptospiral immunoglobulin-like protein by Leptospira interrogans and evaluation of its diagnostic potential in a kinetic ELISA. J. Med. Microbiol. 53:975-984, Palaniappan, R. U., Y. F. Chang, S. S. Jusuf, S. Artiushin, J. F. Timoney, S. P. McDonough, S. C. Barr, T. J. Divers. K. W. Simpson, P. L. McDonough, and H. O. Mohammed. 2002. Cloning and molecular characterization of an immunogenic LigA protein of Leptospira interrogans. Infect. Immun. 70:5924-5930) with model prep cell (BioRad. CA).

The purified protein was dialyzed at 4° C. with PBS by changing PBS for at least 4 times. The purified protein was concentrated and lyophilized (Palaniappan, R. U., Y. F. Chang, F. Hassan, S. P. McDonough, M. Pough, S. C. Barr, K. W. Simpson, H. O. Mohammed, S. Shin, P. McDonough, R. L. Zuerner, J. Qu, and B. Roe. 2004. Expression of leptospiral immunoglobulin-like protein by Leptospira interrogans and evaluation of its diagnostic potential in a kinetic ELISA. J. Med. Microbiol. 53:975-984). The lyophilized protein was suspended with sterile PBS and quantified by the Bradford assay (Bio-Rad).

SDS-PAGE and western blotting. SDS-PAGE was performed as previously described (Chang, Y. F. D. P. Ma, J. Shi, and M. M. Chengappa. 1993. Molecular characterization of a leukotoxin gene from a Pasteurella haemolytica-like organism, encoding a new member of the RTX toxin family. Infect. Immun. 61:2089-2095). Equal amounts of recombinant proteins (1.5-2 μg) were loaded into an SDS-PAGE gel and the gel was stained with Coomassie brilliant blue R-250 followed by destaining. For immunoblotting, recombinant proteins were separated by running SDS-PAGE followed by transfer onto a nitrocellulose membrane (Schleicher & Schuell, Keene, N.H.) in a Trans Blot Cell (Bio-Rad Laboratories, Hercules, Calif.). After transfer, the membrane was blocked with TBS plus 1% bovine serum albumin (BSA) for 1 h. Antisera were diluted as shown in FIGS. 3A-3B in TBS-BSA and were incubated with the blot for 1 h. After three washings in TBS plus 0.1% Tween 20, the blot was incubated for 1 h with 1:2,000 goat anti-bovine IgG conjugated with alkaline phosphatase (KPL). The blot was washed three times as described above and developed with NBT/BCIP as described by Chang et al. (1995. Recombinant OspA protects dogs against infection and disease caused by Borrelia burgdorferi. Infect. Immun. 63:3543-3549).

6.1.3 Results

Identification, expression and purification of outer membrane proteins. The genomic sequences of L. interrogans serovar Icterohaemorrghiae encode for 131 putative outer membrane proteins are known in the art (see, for example, the listings at website address: www.chgc.org.cn). 15 outer membrane proteins, including MceI and II, that did not show high homology to proteins of other pathogens within the NCBI database were randomly selected from the available genomic sequences of Leptospira. These 15 OMPs show 98-100% homology with the genomes from L. interrogans serovar Pomona and L. interrogans serovar Copenhageni.

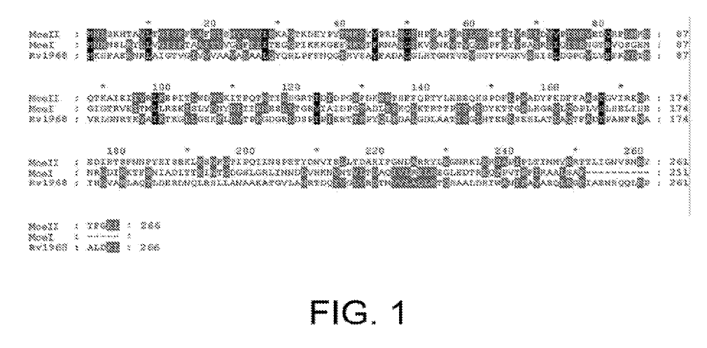

FIG. 1 shows the homology of hypothesized mammalian cell invasion proteins of Leptospira. The top sequence is MceII, a hypothetical MceI-like protein of L. interrogans serovar Pomona. This protein was renamed “MceII” (Lp0607, SEQ ID NOs: 7-8) to distinguish it from the MceI of L. interrogans serovar Lai (middle sequence). The middle sequence is Mce I of L. interrogans serovar Lai (GenBank Accession number AAN49254; SEQ ID NO: 12). The bottom sequence is amino acids 31-293 of Rv1968, an Mce of Mycobacterium tuberculosis CDC1551 (GenBank accession number AE000516, SEQ ID NOS: 13-14). The top sequence, MceII, has approximately 30% homology with the middle sequence, MceI of L. interrogans serovar Lai, and approximately 25-30% homology with the bottom sequence, Rv1968 Mce of Mycobacterium tuberculosis.

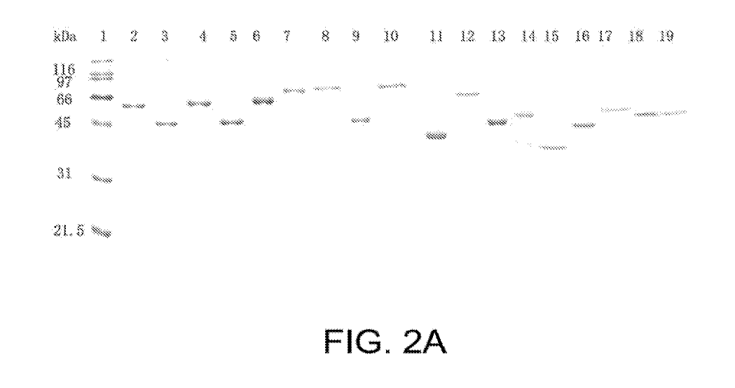

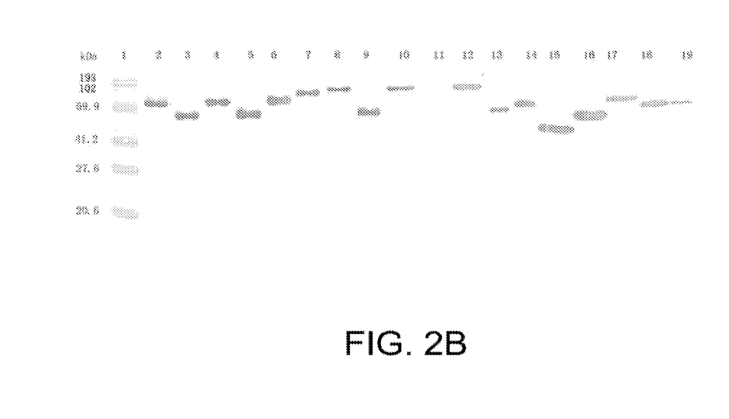

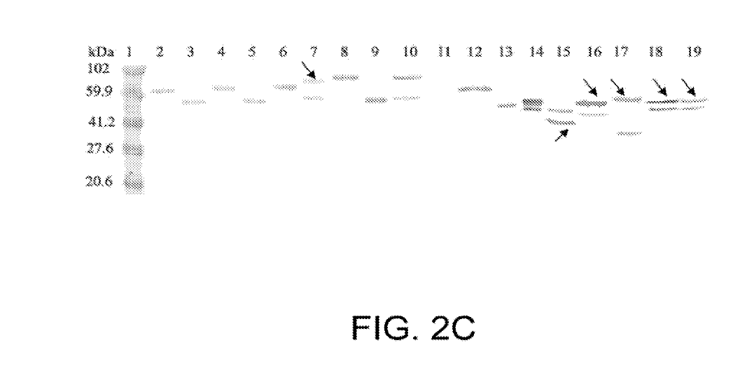

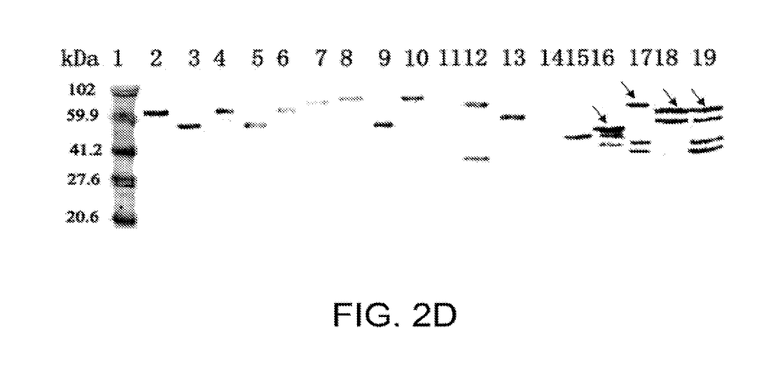

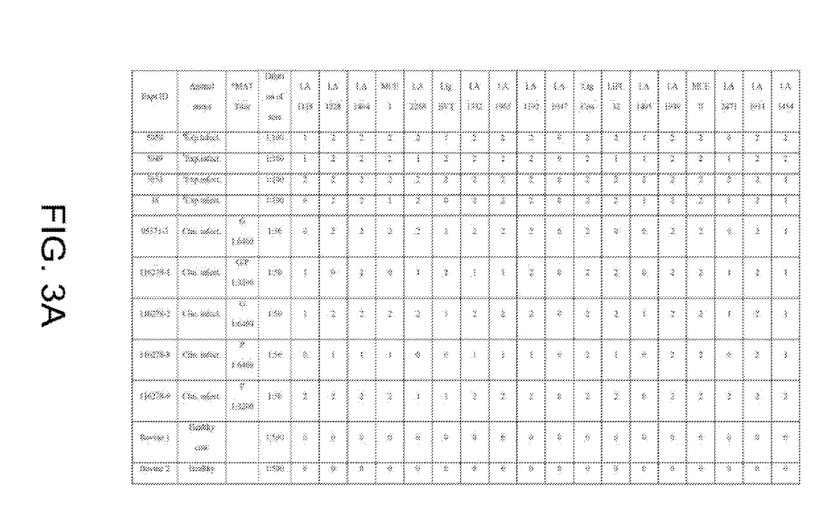

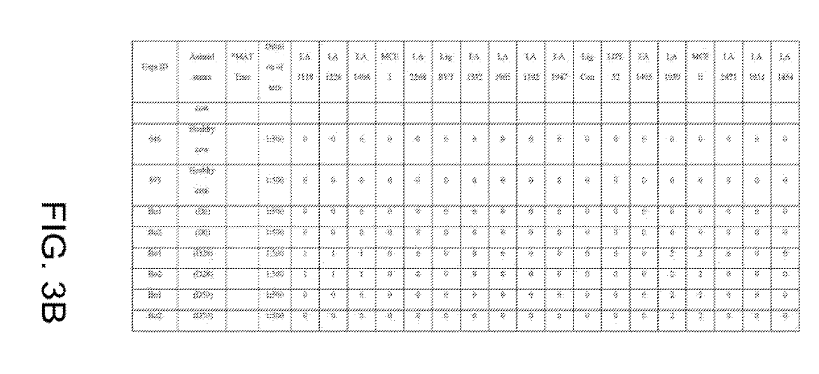

All these genes encoding OMPs and in addition, LigBVT (variable regions of LigB). LigCon (conserved region of LigA and B) and LipL32, were successfully expressed in E. coli as glutathione-S-transferase (GST) fusion proteins. These recombinant proteins migrated approximately to the expected molecular weight (FIG. 2A). The results were further confirmed by immunoblotting using polyclonal antibodies to GST (FIG. 2B). FIGS. 3A-3B list the number of base pairs and calculated molecular size for each OMP protein of Leptospira.

Analysis of immunogenicity of fusion proteins with bovine sera from natural infection, experimental infection and healthy cows. Bovine sera from experimentally infected, naturally infected and healthy cows were evaluated for reactivity with whole cell proteins of serovar Pomona. The experimentally infected and naturally infected sera showed reactivity to whole cell antigens, whereas the sera from healthy cows did not. These sera were also tested for reactivity with GST protein and none of them showed reactivity to GST (data not shown).

To determine the immunogenicity of recombinant proteins specifically to infection with L. borgpetersenii serovar Hardjo, L. interrogans serovar Grippotyphosa. L. interrogans serovar Copenhagenii and L. interrogans serovar Canicola, the immunoblot with purified recombinant outer membrane proteins was probed with sera from experimentally infected cattle. Except for recombinant protein LA1947, all the recombinant OMPs showed reactivity to the experimental infection (FIG. 2C). However, LigCon, LipL32, LA1939 and MceII showed stronger reactivity than other recombinant proteins (FIGS. 3A-3B).

To examine the immunogenicity of recombinant OMPs to natural infection, sera from naturally infected cows with different MAT titers were also evaluated. The recombinant proteins reacted variably to naturally infected sera (For example, see FIG. 2D). However, LigCon, LipL32, LA1939 and MceII showed consistent reactivity to all the naturally infected sera (FIGS. 3A-3B). Control sera from healthy cattle exhibited no antibodies to the recombinant fusion proteins (data not shown).

Sera from cattle experimentally infected with L. borgpetersenii serovar Hardjo during time course infection studies (Day0, Day28, Day50) were also evaluated for reactivity to the recombinant OMPs. As discussed above, LA1939 and MceII showed stronger reactivity than other recombinant proteins (FIGS. 3A-3B). These preliminary data indicate that the proteins encoded by LP1939 and MceII may serve as potential diagnostic antigens or vaccines for leptospiral infection, apart from LigA/B.

6.1.4 Discussion

Leptospirosis has emerged as a globally important infectious disease. It occurs in urban as well as in rural regions of industrialized and developing countries (Levett, P. N. 2001. Leptospirosis. Clin. Microbiol. Rev. 14:296-326). Mortality and infection rates remains significant and are related to delays in diagnosis, lack of infrastructure and adequate clinical suspicion, and also other poorly understood reasons that may include inherent pathogenicity of some leptospiral strains or genetically determined host immunopathological responses. Diagnostic tools are greatly needed that accurately identify infected animals, and thus, play a vital role in preventing the disease. A highly specific test that could distinguish between infected and noninfected animals within herds can have a greater and immediate impact on the dairy industry than any other current management approach, including vaccination (Masuzawa, T., T. Matsumoto, R. Nakamura, R. Suzuki, T. Shimizu, and Y. Yanagihara. 1990. Protective activity of glycolipid antigen against infection by Leptospira interrogans serovar canicola. J. Gen. Microbiol. 136:327-330).

Because of a low index of clinical manifestations, various kinds of diagnostic methods are currently used, such as culture, PCR assays, microscopic agglutination tests (MAT) and other serological assays (Matsuo, K., E. Isogai, and Y. Araki. 2000. Control of immunologically crossreactive leptospiral infection by administration of lipopolysaccharides from a nonpathogenic strain of Leptospira biflexa. Microbiol. Immunol. 44:887-890). The drawbacks of culture and isolation of leptospires from clinical specimens are that they are insensitive and slow. A real-time quantitative PCR assay using TaqMan chemistry to detect leptospires in clinical and environmental samples has been reported (Palaniappan et al. 2005. Evaluation of lig-based conventional and real time PCR for the detection of pathogenic leptospires. Mol. Cell Probes 19:111-117). This PCR assay is sensitive and can differentiate between pathogenic and non-pathogenic species. However, it needs advanced instruments and is difficult to apply widely.

Serology is currently the most frequently used diagnostic approach for leptospirosis. The microscopic agglutination test (MAT) is the standard test for serological diagnosis of leptospires because of its high sensitivity and specificity (Murray, R. D. 1990. A field investigation of causes of abortion in dairy cattle. Vet. Rec. 127:543-547). The MAT detects agglutinating antibodies in serum, but requires significant expertise from its users, and interlaboratory variation in results is high. Whole cell antigen-based diagnostic tests for leptospirosis contain undefined antigens that can exhibit cross-reactivity with other bacteria. Therefore, recombinant protein-based, and especially OMP-based, diagnostic tests may serve as an ideal tool for diagnosis of leptospirosis.

To develop more-reliable antigen-based tests such as ELISAs, identification of leptospiral antigens that are expressed or upregulated only during infection was performed in the present study. With the available genomic sequences for Leptospira, 15 OMPs of Leptospira were chosen and were cloned and expressed in E. coli. These randomly selected OMPs do not show high homology with other proteins from other pathogens in NCBI database. Analyses of antigenicity of OMPs indicate that all the recombinant proteins except LA1947 showed immunoreactivity to experimentally infected cattle. Sera from cattle experimentally infected during a time course infection study reacted strongly to LA1939 and MceII. Further evaluation of recombinant OMPs with naturally infected cattle also confirmed the immunogenicity of recombinant OMPs of LA1939 and MceII. Therefore, LA1939 and MceII can serve as potential diagnostic antigens.

An E. coli expression system with GST fusion proteins for OMPs of Leptospira was also developed in this study. Stable expression of intact antigen was optimal only with GST fusion protein. The GIST affinity tag used in this study has distinct advantages (Deutz, A., K. Fuchs, N. Nowotny, H. Auer, W. Schuller, D. Stunzner, H. Aspock, U. Kerbl, and J. Kofer. 2003. Sero-epidemiological studies of zoonotic infections in hunters—comparative analysis with veterinarians, farmers, and abattoir workers. Wien Kiln Wochenschr 115 Suppl 3:61-67). Generally, expression of recombinant proteins as a fusion protein with GST can overcome problems such as toxicity in E. coli, low expression levels, inclusion body formation, and insolubility. The disadvantage of GST is the size of the affinity tag, which complicates any downstream immunoassays. The lack of reactivity of sera from experimentally and naturally infected cattle, as well as healthy cows, to rGST confirmed the absence of antibodies to GST proteins in these samples. However, the development of immunoassays without GST can be done to evaluate the utilization of these recombinant antigens in the field samples.

Currently available diagnostic test cannot discriminate vaccination and infection. Apparently, the administration of whole cell antigen vaccines would definitely increase the titer of antibodies to OMPs and other proteins. Therefore, targeting the proteins that are upregulated during infection can be used to develop immunoassays to discriminate infection and vaccination. The recombinant OMPs of LP1939 and MceII can serve, therefore, as an ideal diagnostic tool to identify infection, and further evaluation of these antigens with vaccinated and infected animals can be performed.

In a conclusion, 15 putative OMPs of Leptospira were randomly selected, cloned and expressed in E. coli. The recombinant proteins were evaluated for potential use in diagnosis of leptospirosis. Out of 15 recombinant proteins, LP1939 and MceII showed consistently stronger reactivity to sera from experimentally and naturally infected animals.

### 6.2 Example 2

### Immunogenicity of Recombinant Leptospiral Outer Membrane Proteins and their Uses as Vaccine Candidates

6.2.1 Introduction

Leptospiral outer membrane proteins (OMPs) can be components of effective vaccines for leptospirosis. Genomic sequencing of Leptospira has allowed us to target putative OMPs for the development of recombinant vaccines. We focused on 12 OMPs that had no homology with other organisms listed in the NCBI database except MceI and MceII of Leptospira. All putative OMPs were cloned, expressed and purified as glutathione-S-transferase (GST) fusion proteins. Primary screening for immunoprotective potential was performed in hamsters challenged with an LD50 inoculum of low passage serovar Pomona. The fusion proteins rLP1454, rLP1118 and rMCEII were protective when administered to hamsters, as compared to rGST, which was administered as the negative control. All these recombinant proteins were evaluated again for protective efficacy based on lethality, histopathological lesions and antibody responses, rLP1454, rLP1118 and rMCEII showed protection as far as lethality is concerned. Histopathological analyses of rLP1454, rLP1118 and rMCEII immunized animals only showed minor renal lesions, in contrast to rGST-immunized animals (control group), which showed serious lesions. To improve protective efficacy, three recombinant protective antigens were evaluated in combination with rGST and rLigA as negative and positive control respectively. The results indicate that rLP1454, rLP1118, and rMCEII showed protection individually and synergistically against serovar Pomona infection, which can be used to develop a multicomponent vaccine for leptospirosis.

Leptospirosis is a world-wide zoonotic disease caused by gram-negative spirochetes belonging to the genus Leptospira. People who contract leptospirosis, either directly through contact with infected animals or indirectly from a contaminated environment, often develop kidney and liver failure (Bain et al. 1973. Renal failure and transient paraproteinemia due to Leptospira pomona. Case reports and literature review. Arch. Intern. Med. 131:740-745; Divers, et al. 1992. Renal dysfunction associated with infection of Leptospira interrogans in a horse. J. Am. Vet. Med. Assoc. 201:1391-1392; Garcia Garcia, et al. 1979. Icterohemorrhagic leptospirosis with acute renal failure (author's transl). Med. Clin. (Bare) 73:362-366; Kager, et al. 2001. Fever and chills due to leptospirosis after travel to Thailand. Ned. Tijdschr. Geneeskd. 145:184-186; Menzies, et al. 1989. Leptospira icterohaemorrhagiae infection presenting as acute renal failure. Scott Med. J. 34:410; Petros, et al. 2000. Serum procalcitonin and proinflammatory cytokines in a patient with acute severe leptospirosis. Scand. J. Infect. Dis. 32:104-105; San Segundo, et al. 1982. Leptospirosis caused by L. grippotyphosa accompanied by acute renal failure (author's transl). Med. Clin. (Bare) 78:28-31; Schubert, et al. Acute renal failure in leptospirosis (with a report on the differential diagnosis of Leptospira sejroe and Leptospira icterohaemorrhagiae), Munch. Med. Wochenschr. 113:80-86; Winearls, et al. 1984. Acute renal failure due to leptospirosis: clinical features and outcome in six cases. Q. J. Med. 53:487-495). Infection in horses, cattle, dogs and pigs can cause abortion, still birth, renal failure, and uveitis (Akkermans, J. P. 1966. Incidence of abortion and sterility in swine in the Netherlands due to infection with Leptospira hyos. Bull. Off. Int. Epizoot. 66:849-866; Andreani, E., F. Tolari, and R. Farina. 1983. Experimental infection in sheep with Leptospira interrogans serotype hardjo. Br. Vet. J. 139:165-170; Bernard, W. V. 1993. Leptospirosis Vet. Clin. North Am. Equine Pract. 9:435-444; Bolin, C. A., J. A. Cassells, H. T. Hill, J. C. Frantz, and J. N. Nielsen. 1991. Reproductive failure associated with Leptospira interrogans serovar Bratislava infection of swine. J. Vet. Diagn. Invest. 3:152-154; Broll, S., A. S. Waldvogel, M. Rosskopf, L. Corboz, and A. Pospischil. 1993. The infectious causes of abortion and stillbirth in swine in Switzerland. Zentralbl. Veterinarmed. B 40:641-653; Donahue, J. M., B. J. Smith, J. K. Donahoe, C. L. Rigsby, R. R. Tramontin, K. B. Poonacha, and M. A. Wilson, 1992. Prevalence and serovars of leptospira involved in equine abortions in central Kentucky during the 1990 foaling season. J. Vet. Diagn. Invest. 4:279-284; Donahue, J. M., B. J. Smith, K. B. Poonacha, J. K. Donahoe, and C. L. Rigsby, 1995. Prevalence and serovars of leptospira involved in equine abortions in central Kentucky during the 1991-1993 foaling seasons. J. Vet. Diagn. Invest. 7:87-91: Donahue, J. M., B. J. Smith, K. J. Redmon, and J. K. Donahue. 1991 Diagnosis and prevalence of leptospira infection in aborted and stillborn horses. J. Vet. Diagn. Invest. 3:148-151; Donahue, J. M., and N. M. Williams, 2000. Emergent causes of placentitis and abortion. Vet. Clin. North Am. Equine Pract. 16:443-456, viii; Eider, J. K., P. M. Pepper, M. W. Hill, and W. H. Ward. 1985. The significance of leptospiral titres associated with bovine abortion. Aust. Vet. J. 62:258-262; Ellis, T. M., G. M. Robertson, L. Hustas, and M. Kirby, 1983. Detection of leptospires in tissue using an immunoperoxidase staining procedure. Aust. Vet. J, 60:364-367; Ellis, W. A., D. G. Bryson, and J. B. McFerran. 1976. Abortion associated with mixed Leptospira/equid herpesvirus 1 infection. Vet. Rec. 98:218-219; Rocha, T. 1990. Isolation of Leptospira interrogans serovar mozdok from aborted swine fetuses in Portugal. Vet. Rec. 126:602) and can even lead to multi-organ failure.

Currently available whole cell leptospiral vaccines generally stimulate only short-term immunity and do not provide cross protection against the 250 known serovars of pathogenic Leptospira. Furthermore Leptospira infection can trigger autoimmune disease in horses and people (Parma, A. E., S. I. Cerone, and S. A. Sansinanea. 1992. Biochemical analysis by SDS-PAGE and western blotting of the antigenic relationship between Leptospira and equine ocular tissues. Vet. Immunol. Immunopathol. 33:179-185; Parma, A. E., S. I. Cerone, S. A. Sansinanea, and M. Ghezzi. 1992. C3 fixed in vivo to cornea from horses inoculated with Leptospira interrogans. Vet. Immunol. Immunopathol. 34:181-187; Parma, A. E., A. S. Fernandez, C. G. Santisteban, R. A. Bowden, and S. I. Cerone, 1987. Tears and aqueous humor from horses inoculated with Leptospira contain antibodies which bind to cornea. Vet. Immunol. Immunopathol. 14:181-185; Parma, A. E., M. E. Sanz, P. M. Lucchesi, J. Mazzonelli, and M. A. Petruccelli. 1997, Detection of an antigenic protein of Leptospira interrogans which shares epitopes with the equine cornea and lens. Vet. J. 153:75-79; Rathinam. S. R., S. Rathnam, S. Selvaraj, D. Dean, R. A. Nozik, and P. Namperumalsamy. 1997. Uveitis associated with an epidemic outbreak of leptospirosis. Am. J. Ophthalmol. 124:71-79. Thus, the safety and efficacy of leptospiral vaccines may be improved by administering immunoprotective subunits rather than administering whole cell antigens.

The conventional approach to vaccine development depends on the identification of protective antigens for use as subunit vaccines (Rappuoli, R., and G. Del Giudice. 1999. Identification of vaccine targets. CRC, Boca Raton). Study of leptospiral extracts indicates that outer membrane components contain protective antigens. Importantly, some of these protective OMPs are expressed at only low levels or may not be expressed at all by leptospires grown in artificial media (Palaniappan, R. U., Y. F. Chang, S. S. Jusuf, S. Artiushin, J. F. Timoney, S. P. McDonough, S. C. Barr, T. J. Divers, K. W. Simpson, P. L. McDonough, and H. O. Mohammed. 2002. Cloning and molecular characterization of an immunogenic LigA protein of Leptospira interrogans. Infect. Immun. 70:5924-5930). Various leptospiral OMPs such as OmpL1, LipL41, LipL32, LipL36, Lig and LipL21 have been cloned and characterized (Cullen, P. A., S. J. Cordwell, D. M. Bulach, D. A. Haake, and B. Adler. 2002. Global analysis of outer membrane proteins from Leptospira interrogans serovar Lai. Infect. Immun. 70:2311-231.8; Haake, D. A. 2000. Spirochaetal lipoproteins and pathogenesis. Microbiology 146:1491-1504; Haake, D. A., C. Martinich, T. A. Summers, E. S. Shang, J. D. Pruetz, A. M. McCoy, M. K. Mazel, and C. A. Bolin. 1998. Characterization of leptospiral outer membrane lipoprotein LipL36: downregulation associated with late-log-phase growth and mammalian infection. Infect. Immun. 66:1579-1587; Palaniappan, R. U., Y. F. Chang, F. Hassan, S. P. McDonough, M. Pough, S. C. Barr, K. W. Simpson, H. O. Mohammed, S. Shin, P. McDonough, R. L. Zuerner, J. Qu, and B. Roe. 2004. Expression of leptospiral immunoglobulin-like protein by Leptospira interrogans and evaluation of its diagnostic potential in a kinetic ELISA. Med. Microbiol. 53:975-984; Palaniappan, R. U., Y. F. Chang, S. S. Jusuf, S. Artiushin. J. F. Timoney, S. P. McDonough, S. C. Barr, T. J. Divers, K. W. Simpson, P. L. McDonough, and H. O. Mohammed. 2002. Cloning and molecular characterization of an immunogenic LigA protein of Leptospira interrogans. Infect. Immun. 70:5924-5930: Palaniappan, R. U.S. P. McDonough. T. J. Divers, C. S. Chen, M. J. Pan, M. Matsumoto, and Y. F. Chang. 2006. Immunoprotection of recombinant leptospiral immunoglobulin-like protein A against Leptospira interrogans serovar Pomona infection. Infect. Immun. 74:1745-1750; Shang, E. S., M. M. Exner, T. J. Summers, C. S. Martinich, C. I. Champion, R. E. Hancock, and D. A. Haake. 1995. The rare outer membrane protein, OmpL1, of pathogenic Leptospira species is a heat-modifiable porin. Infect. Immun. 63:3174-3181). LipL32 protects animals when used in a DNA vaccine formula, but not as a recombinant protein (Branger, C., B. Chatrenet. A. Gauvrit, F. Aviat, A. Aubert, J. M. Bach, and G. Andre-Fontaine. 2005. Protection against Leptospira interrogans sensulato challenge by DNA immunization with the gene encoding hemolysin-associated protein 1. Infect. Immun. 73:4062-4069). In contrast, the recombinant protein LigA is able to induce immunoprotection against leptospirosis (Koizumi, N., and H. Watanabe. 2003. Identification of a novel antigen of pathogenic Leptospira spp. that reacted with convalescent mice sera. J. Med. Microbiol. 52:55-589; Palaniappan, R. U., S. P. McDonough, T. J. Divers, C. S. Chen, M. J. Pan, M. Matsumoto, and Y. F. Chang, 2006. Immunoprotection of recombinant leptospiral immunoglobulin-like protein. A against Leptospira interrogans serovar Pomona infection. Infect. Immun. 74:1745-1750).

Although results of protection experiments in hamsters and mice with Lig proteins are promising, a combination of key antigens and/or epitopes is likely to be more efficacious against multiple leptospira serovars, especially in outbred animals. Such a multicomponent vaccine circumvents the problems of host genetic restriction and antigenic variability associated with single antigen-based vaccines.

Recently, vaccine development has taken advantage of the genome sequence of pathogenic bacteria (Kelly, D. F., and R. Rappuoli. 2005. Reverse vaccinology and vaccines for serogroup B Neisseria meningitidis. Adv. Exp. Med. Biol. 568:217-223; Rappuoli, R. 2000. Reverse vaccinology. Curr. Opin. Microbiol, 3:445-450; Rappuoli, R. 2001. Reverse vaccinology, a genome-based approach to vaccine development. Vaccine 19:2688-2691; Rappuoli, R., and A. Covacci. 2003. Reverse vaccinology and genomics. Science 302:602).

This approach not only identifies all the antigens described by conventional methods, but also enables the discovery of novel antigens. Since OMPs are the primary bacterial components that interact with host cells, targeting the OMPs for development of recombinant vaccine is advantageous.

In this study, we focused on 13 OMPs that had no homology with other bacterial proteins listed in NCBI database as well as Leptospira MceI and II. All these OMPs were cloned, expressed and purified as GST fusion proteins. Screening for protective efficacy of recombinant OMPs was performed in a hamster model. Of these, rMceII, rLP1118 and rLP1454 serve as immunoprotective antigens against L. interrogans serovar Pomona infection. All these recombinant antigens were evaluated separately as well as in combination along with rLigA (positive control) and rGST (negative control) suggesting that rMceII, rLP1118 and rLP1454 could serve as protective antigens individually and synergistically paving the way for the development of a multicomponent vaccine against leptospirosis.

6.2.2 Materials and Methods

Bacterial strains, media and plasmids. L. interrogans serovar Pomona was used as previously described (Palaniappan, et al. 2002. Cloning and molecular characterization of an immunogenic LigA protein of Leptospira interrogans. Infect. Immun. 70:5924-5930). Leptospires were maintained on EMJH medium at 30° C. (Palaniappan, et al. 2002. Cloning and molecular characterization of an immunogenic LigA protein of Leptospira interrogans, Infect. Immun. 70:5924-5930). To isolate low passage cultures of leptospires, hamsters were experimentally infected with a sublethal dose of L. interrogans serovar Pomona (NVSL 1427-35-093002). Infected hamster tissues were harvested aseptically, homogenized with sterile PBS, and the lysates were inoculated into EMJH medium. Growth was monitored using dark field microscopy.

LD₅₀ value of L. interrogans in hamster. Twenty-four 8-9 week-old Golden Syrian hamsters (Harlan Sprague Dawley) were divided into 4 equal groups. Each group was infected intraperitoneally with 10⁹ to 10⁷ low passage (3-4 passages) L. interrogans serovar Pomona (NVSL 1427-35-093002). One group of hamsters received only PBS (control). The animals were monitored daily. Tissues were collected aseptically from animals that died due to infection and subjected to culture and histopathological analysis.

OMP cloning and expression. Primers for the putative outer membrane were designed without the signal sequences and are listed in Table 1 (Section 6.1). Genomic DNA of L. interrogans serovar Pomona was prepared using a DNAeasy kit (Qiagen, Valencia, Calif.) and subjected to PCR as described by the manufacturer using Accuprime Taq polymerase (Invitrogen, CA). PCR products were run in 1% agarose gel and visualized by ethidium bromide staining, PCR products were eluted from the gel using gel elution kit (Qiagen, Valencia, Calif.) and cloned into TOPO TA Vector as described by the manufacturer (Invitrogen. CA). The insert was then sub-cloned into the expression vector pGEX-KGC (Stratagene). LP1454, 1118 and 0607(MCEII) were also inserted into pRSETA (InVitroGen, CA) to express recombinant proteins for serologic test. DNA sequencing was analyzed with an ABI model 377 automated nucleic acid sequencer at the Bioresource Center, Cornell University. Homology searches were performed with the NCBI database and BLAST (Altschul, S. F., W. Gish, W. Miller, E. W. Myers, and D. J. Lipman. 1990. Basic local alignment search tool. J. Mol. Biol. 215:403-410).

Expression and purification of recombinant proteins. Exponentially growing cultures (OD₆₀₀=0.6) of E. coli (BL21 DE3) or CQ21 harboring recombinant plasmids were induced with or without 1 mM isopropyl-β-D-thiogalactopyranoside (IPTG; Sigma, St. Louis, Mo.). For each recombinant protein, a pilot experiment was performed with 3 mL culture volume and the molecular weight of the expressed recombinant proteins was checked by SDS-PAGE analysis. Mass cultivation was performed in 1,000 mL of LB broth with ampicillin (50 μg/mL). Bacteria were harvested by centrifugation at 5000 RPM for 30 min. The cell pellets was washed and suspended in PBS and subjected to a French pressure cell (American Instruments). The lysates were centrifuged at 8,000×g for 30 min and the inclusion bodies were washed and purified as mentioned previously (Palaniappan, et al. 2004. Expression of leptospiral immunoglobulin-like protein by Leptospira interrogans and evaluation of its diagnostic potential in a kinetic ELISA. J. Med. Microbiol. 53:975-984; Palaniappan, et al. 2002, Cloning and molecular characterization of an immunogenic LigA protein of Leptospira interrogans. Infect. Immun. 70:5924-5930) with model prep cell (BioRad, CA). The purified protein was dialyzed at 4° C. with PBS. The purified protein was concentrated and lyophilized (Palaniappan, et al. 2004. Expression of leptospiral immunoglobulin-like protein by Leptospira interrogans and evaluation of its diagnostic potential in a kinetic ELISA. J. Med. Microbiol, 53:975-984). The lyophilized protein was suspended with sterile PBS and quantified using Bio-Rad protein, assay.

SDS-PAGE. SDS-PAGE was performed as previously described (Chang, et al. 1993. Molecular characterization of a leukotoxin gene from a Pasteurella haemolytica-like organism, encoding a new member of the RTX toxin family. Infect. Immun, 61:2089-2095). The equal amount of recombinant proteins (1.5 μg) was subjected to SDS-PAGE and the gel was stained with Coomassie brilliant blue R-250 followed by de-staining (Chang, et al. 1993. Molecular characterization of a leukotoxin gene from a Pasteurella haemolytica-like organism, encoding a new member of the RTX toxin family. Infect. Immun. 61:2089-2095).

Immunization and challenge experiments. Preliminary screening of recombinant putative outer membrane proteins was performed in a group of 4 week-old Golden Syrian hamsters (Harlan Sprague Dawley; 3 hamsters per group). Hamsters were immunized twice by subcutaneous injection with recombinant proteins (50 μg) at an interval of three weeks (Day 0 and Day 21). Hamsters receiving rGST were considered a control group. Prior to immunization, the recombinant proteins or rGST were mixed with an equal volume of aluminum hydroxide (Alhydrogel, Accurate Chemical & Scientific Corp, Westbury, N.Y.). Blood for serum separation was collected on days 0 (pre-vaccination), 21 and 42 directly from the saphenous vein.

Immunized hamsters were challenged intraperitoneally with the estimated LD₅₀ value of 10⁸ leptospires on D42. The animals were monitored twice daily, sacrificed on day 71 and blood was collected by cardiac puncture. The tissues from infected animals were collected aseptically for histopathological analysis and culture.

Recombinant protective antigens (rLP1454, rLP1118 and rMCEII) were analyzed again as discussed above in a group of hamster (8 animals per group). Animals immunized with rGST and rLigA were considered as negative and positive controls, respectively. The protective efficacy of the three recombinant antigens in combination (50 μg of each antigen) was assessed in a group of hamsters (8 hamsters per group) with rGST and rLigA considered as negative and positive controls, respectively.

Culture. Hamster tissues such as liver, kidney, spleen, lungs, urinary bladder and blood were collected aseptically from each animal, homogenized in 0.5 ml EMJH medium, transferred into 20 ml EMJH medium and maintained at 30° C. for 4 weeks (Palaniappan, et al. 2002. Cloning and molecular characterization of an immunogenic LigA protein of Leptospira interrogans. Infect. Immun. 70:5924-5930: et al. 2006. Immunoprotection of recombinant leptospiral immunoglobulin-like protein A against Leptospira interrogans serovar Pomona infection. Infect. Immun. 74:1745-1750). Growth was monitored using dark field microscopy.

Histopathology. Hamster tissues were fixed by immersion in 10% neutral buffered formalin, embedded in paraffin, sectioned at 5 μm, stained with hematoxylin and eosin (H&E) using standard histological techniques, and examined by light microscopy. The severity of Leptospira induced tubulointerstitial nephritis was graded by a veterinary pathologist (SPMcD) who was blind to the identity of the treatment groups and who graded on the following scale: 0=normal, 1=mild, 2=moderate, and 3=severe.

Mild renal lesions had interstitial infiltrates of small numbers of lymphocytes occasionally mixed with a few heterophils and macrophages. Occasional tubules were mildly dilated and distended with hyaline material that contained rare sloughed epithelial cells. The lining epithelium was also moderately attenuated. Rarely, desquamated epithelial cells and heterophils formed mixed casts with hyaline material within the lumina of proximal convoluted. Moderate renal lesions had prominent perivascular accumulations of lymphocytes mixed with lesser number of plasma cells, macrophages and occasional heterophils. Tubular changes were more common than in mildly affected kidneys and hyaline casts were noted occasionally. Severely affected kidneys were irregularly shrunken with widespread interstitial fibrosis accompanied by extensive degeneration and loss of proximal convoluted tubules. Hyaline casts were numerous but glomerular changes were generally mild. Bowman's capsules of glomeruli in regions of severe tubular atrophy and fibrosis were dilated and the uriniferous spaces were filled with a faintly eosinophilic lacy material. Some glomeruli had shrunken glomerular tufts while others had patchy increases in the amount of mesangial matrix. Mild hypertrophy of the parietal epithelium lining Bowman's capsules was also present is a subset of affected glomeruli.

Kinetic Enzyme-Linked Immunosorbent Assay (KELA) with Recombinant Antigens

Sera from hamsters given recombinant antigens and rGST were evaluated for the presence of specific IgG using a KELA assay with recombinant antigens (Palaniappan, et al. 2004. Expression of leptospiral immunoglobulin-like protein by Leptospira interrogans and evaluation of its diagnostic potential in a kinetic ELISA. J. Med. Microbiol. 53:975-984). A checkerboard titration was followed to optimize the concentrations of reagents and the KELA assay was performed as previously described (Koizumi, N., and H. Watanabe. 2003. Identification of a novel antigen of pathogenic Leptospira spp. that reacted with convalescent mice sera. J. Med. Microbiol. 52:585-589; Naiman, et al. 2002. Evaluation of type I immune response in naive and vaccinated animals following challenge with Leptospira borgpetersenii serovar Hardjo: involvement of WC1(+) gammadelta and CD4 T cells, Infect. Immun. 70:6147-6157; et al. 2005. Evaluation of lig-based conventional and real time PCR for the detection of pathogenic leptospires. Mol. Cell. Probes 19:111-117).

Briefly, the optimum concentration of recombinant Histag-fusion proteins (rMceII, rLP1454, and rLP1118) were diluted in 0.1M bicarbonate buffer, coated onto a 96 well microtiter plate (Nunc, Denmark), rocked for 1 hour, and then incubated overnight at 4° C. The plates were washed three times with 0.1M PBS containing 0.05% Tween 20 (PBST). Next, 100 μl of hamster serum (primary antibody) diluted 1:100 in PBST was added to each well and incubated for 1 hour at 37° C. in a humid chamber. The plates were washed three times as described above with PBST and incubated with 100 μl of a 1:1000 dilution of goat anti-hamster IgG conjugated to horseradish peroxidase (Cappel, Durham, N.C.) for 30 nm in at room temperature. The plates were washed again three times with PBST and 100 μl of TMB (Kirkegaard, MD) was added to each well. Each plate was read three times at 650 nm OD with an interval of 1-minute (Biotek EL-312, Winoski, Vt.).

The results were calculated by the KELA computer program (publicly available for use at the Animal Health Diagnostic Center, College of Veterinary Medicine, Cornell University, Ithaca N.Y.) and expressed as the slope of the reaction between the enzyme and the substrate to the amount of antibody bound (Chang et al. 1999. Vaccination against lyme disease with recombinant Borrelia burgdorferi outer-surface protein A (rOspA) in horses. Vaccine 18:540-548; Chang, et al. 1995. Recombinant OspA protects dogs against infection and disease caused by Borrelia burgdorferi. Infect Immun 63:3543-3549).

6.2.3 Results

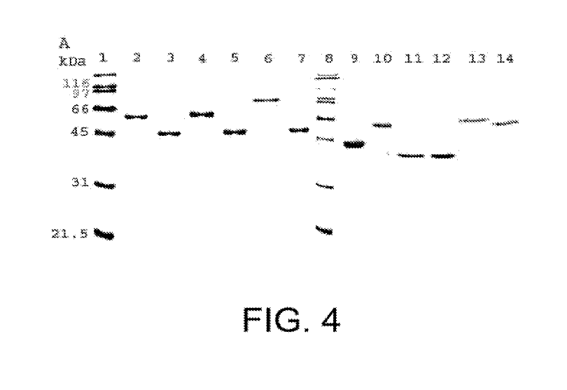

Expression and purification of recombinant putative outer membrane proteins as GST fusion proteins: The genomic sequences of Leptospira predicted several putative OMPs. Of these, 13 putative outer membrane proteins and also MCEI and II were targeted for vaccine development. All these OMPs were cloned and expressed as GST fusion proteins. Purified recombinant GST fusion proteins appeared as a single band by SDS-PAGE, analysis (FIG. 4).

LD50 value for L. interrogans serovar Pomona in a hamster model: Hamsters (8 animals per group) that received 10⁹ suffered 100% mortality. However, 5 of 8 hamsters survived after receiving a dose of 10⁸ leptospires. All the hamsters that received a dose of 10⁷ leptospires survived, indicating the LD₅₀ for L. interrogans serovar Pomona infection was approximately 10⁸.

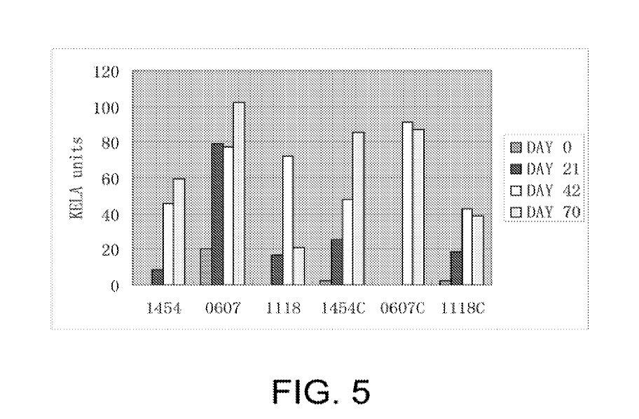

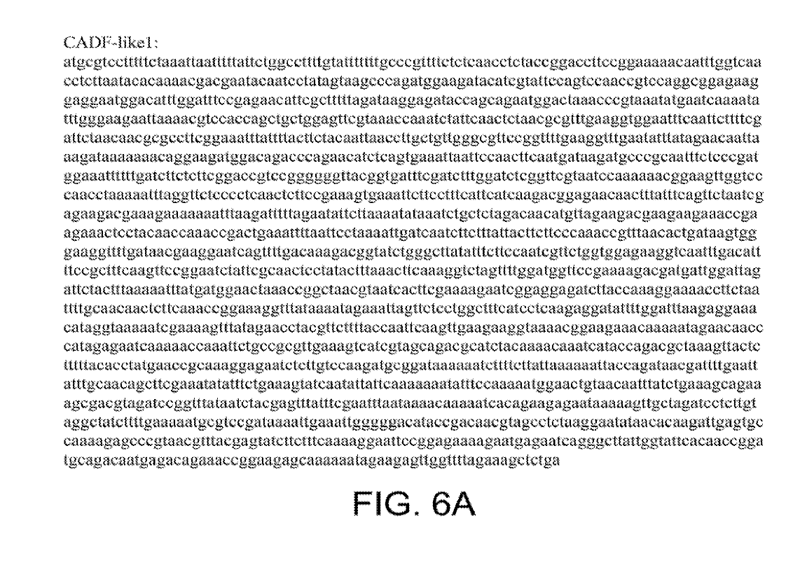

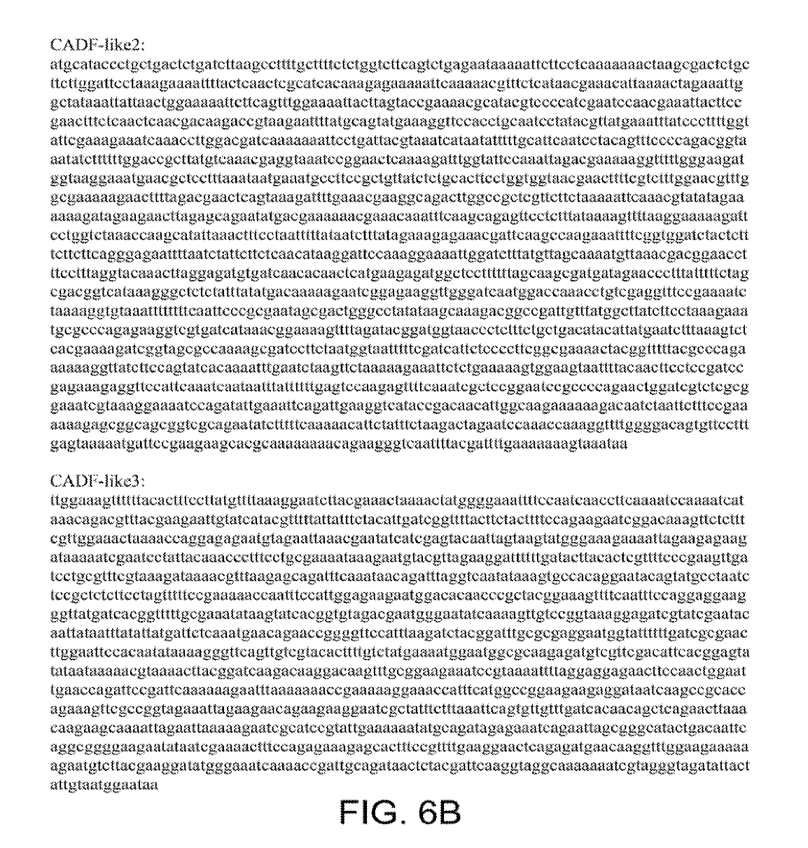

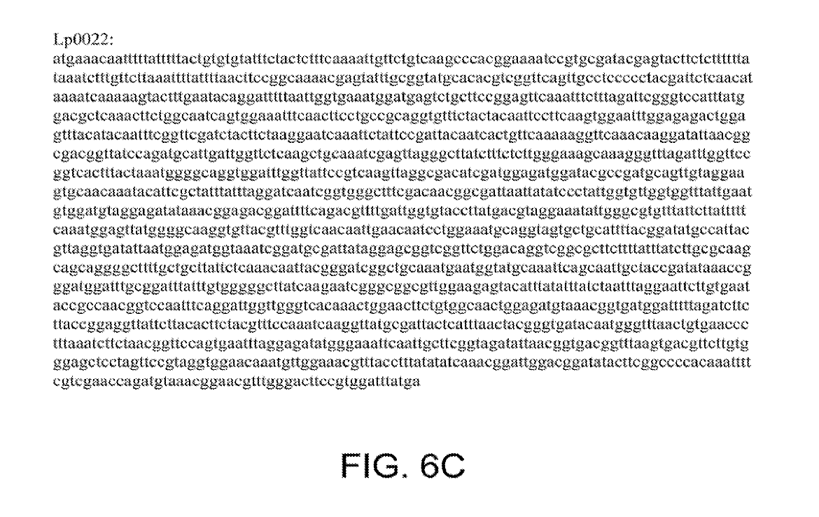

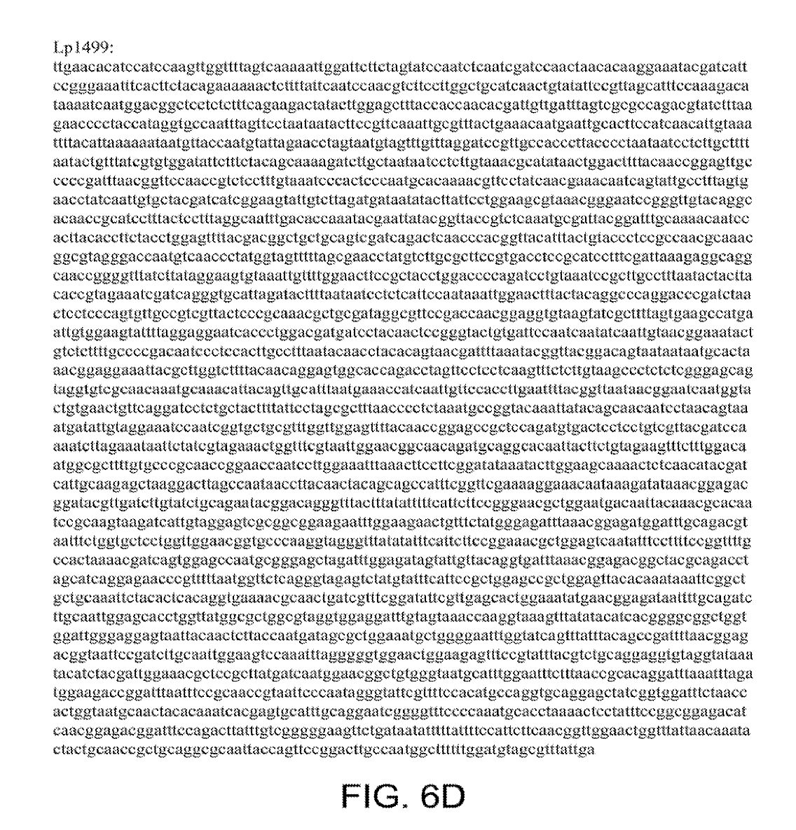

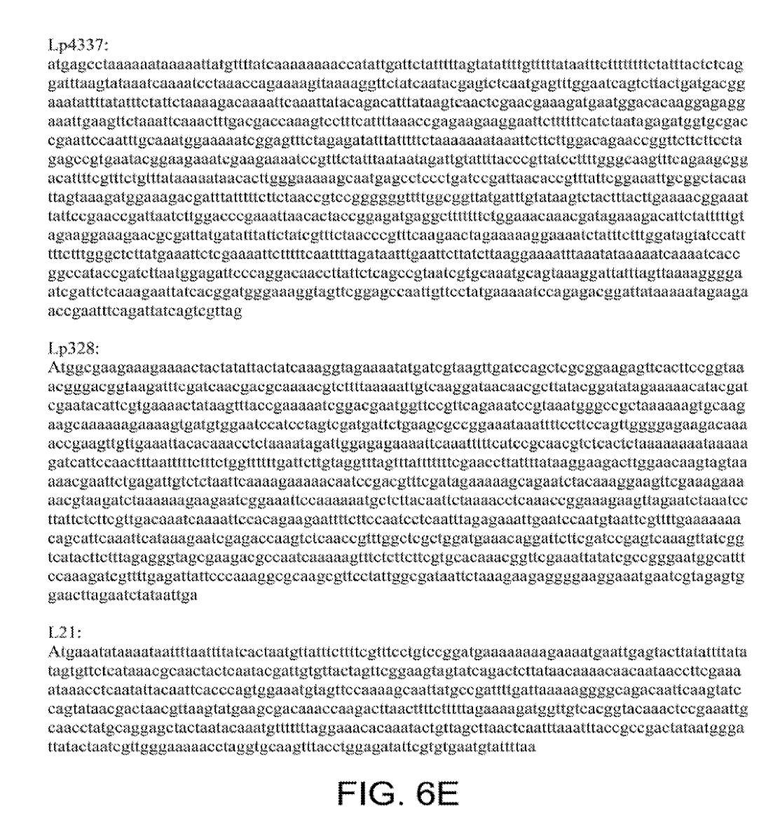

Antibody responses to recombinant antigens: To examine whether hamsters given recombinant antigens developed IgG antibody response against the antigens, hamsters immunized with 50 μg of recombinant proteins (rMCEII, rLP1454 and rLP1118) individually and in combination were evaluated for serum IgG antibody level. Sera from the animals collected on days 0, 21, 42 and 71 were analyzed by KELA using rMceII, r1454, rLP1118, and rLigA. Hamsters showed IgG antibodies to rMceII, r1454, rLP1118 and rLigA, indicating the immune response against recombinant protein (FIG. 5). FIG. 5 shows that serum from immunized animals was collected on D0 (pre-immune) D21 (serum samples after first immunization), D42 (serum samples after second immunization) and D72 (29 days after challenge). FIG. 5 represents hamsters immunized individually with recombinant proteins (rLP1454, rLP0607, and rLP111b) or immunized with combination of recombinant proteins (rLP1454C, rLP0607C, and rLP111bC).

Recombinant antigens rMceII, rLP1118 and r1454 confer protective immunity in a hamster model: Preliminary screening was evaluated in hamsters (3 animals per group) given either recombinant OMP or rGST as a negative control. All the animals given rGST succumbed (Table 2).

TABLE 2
Preliminary screening of protective efficacy of recombinant proteins of
OMPs in a hamster model

| | | No. of surviving animals/total |
|---|---|---|
| No: | Group | no: of animals |
| 1 | rGST (control) | 0/3 (0) |
| 2 | rLA2471 | 0/3 (0) |
| 3 | rLA1454 | 2/3 (66) |
| 4 | rLA1495 | 0/3 (0) |
| 5 | rLA1939 | 0/3 (0) |
| 6 | rLA1965 | 0/3 (0) |
| 7 | rLA1332 | 0/3 (0) |
| 8 | rMCE I | 1/2 (50) |
| 9 | rLA1404 | 1/2 (50) |
| 10 | rLA1228 | 1/3 (33) |
| 11 | rLA1118 | 2/3 (66) |
| 12 | rLA1947 | 1/3 (33) |
| 13 | rMCEII | 2/3 (66) |

However, ⅔ of the hamsters that received rMceII, rLP1118 and r1454 survived after challenge, indicating that these recombinant proteins may serve as protective antigens.

To confirm the protective efficacy of rMceII, rLP1118 and r1454, these recombinant antigens were analyzed again in hamsters (8 hamsters per group) with rLigA and rGST as positive and negative controls, respectively. Compared to the rGST immunized animals, where only 3/7 hamsters survived, 5/7, 6/8, 7/7 and 8/8 hamsters given rLP1454, rLP1118, rMCEII and rLigA, respectively, survived (Table 3).

TABLE 3
Confirmation of protective effect of rLP1454, rLP1118 and rMCEII

| | | No. of surviving animals/total |
|---|---|---|
| No: | Group | no: of animals |
| 1 | rLA1454 | 5/7 (71%) |
| 2 | rLA1118 | 6/8 (75%) |
| 3 | rMCEII | 7/7 (100%) |
| 4 | rGST (control)] | 3/7 (43%) |

Thus, rMceII, rLP1454 and LP1118 provided protection from the lethal effects of L. interrogans serovar Pomona infection.

To evaluate whether these recombinant proteins can enhance protection synergistically, hamsters were given a combination of antigens (rLP1454, rLP1118 and rMCEII) and evaluated against an LD50 inoculum of L. interrogans serovar Pomona. Almost all hamsters that were given the recombinant OMP combination survived (7/8) whereas only 4/8 hamsters survived in the control group (rGST immunized animals). These results confirm the synergistic immunoprotective effect of these recombinant antigens (Table 4).

TABLE 4
Synergistic protective effect of rLP1454, rLP1118 and rMCEII

| | | No. of surviving |
|---|---|---|
| | | animals/total no: of |
| No: | Group | animal |
| 1 | rLP1454-LP1118-MCEII | 7/8 (87.5) |
| | (combination) | |
| 2 | rLigA | 8/8 (100) |
| 3 | rGST (control) | 4/8 (50) |

Histopathological analysis. All but two hamsters that received rGST developed severe tubulointerstitial nephritis (Table 6) while those given rLig were completely protected from severe disease.

TABLE 5
Effect of recombinant outer membrane protein vaccination on the
severity of tubulointerstitial nephritis in hamsters challenged with
L. interrogans serovar Pomona

| | Proportion with Severe (grade 3) |
|---|---|
| Recombinant Antigen | Tubulointerstitial Nephritis* |
| rGST | 85% |
| rLigA | 0% |
| r1454 | 77% p = 0.6188 |
| r1118 | 36% p = 0.0098 |
| rMCEII | 71% p = 0.4102 |
| (r1454 + r1118 + rMCEII) | 25% p = 0.0261 |

*A Chi-Square test for heterogeneity or independence was used to determine the difference in histopathologic lesion of kidney to the control group (GST). Statistical significance was claimed if p ≦ 0.05.

The recombinant OMPs LP1454 and MCE II had only modest protective effects but rLP1118 markedly decreased the incidence of severe tubulointerstitial nephritis. Interestingly, when rLP1454, rLP1118 and rMCE II were given in combination, the protective effects appear to be synergistic, with only 25% of hamsters developing severe renal lesions. A Chi-Square test for heterogeneity or independence was used to determine the difference in histopathologic lesion of kidney to the control group (GST). Statistical significance was claimed if p≦0.05.

Leptospira culture from tissues. Kidney, liver, urinary bladder, lungs and spleen from all hamsters including immunized and control hamsters were evaluated for the presence of leptospires by culture. Except for the control groups that cultured positive in kidney, urinary bladder and/or spleen in each animal, all other vaccinated groups were cultured positive only in two or three animals per group in kidney and/or urinary bladder only.

6.2.4 Discussion

In this study, we focused on putative OMPs of the Leptospira genome to develop a protein-based vaccine for the prevention and/or treatment of leptospirosis.

We have demonstrated the feasibility of developing recombinant vaccines based on OMPs. Considering the increase in serovars of Leptospira and also host restriction of some of the serovars (for example, serovar Hardjo in cattle), it is advantageous to screen more protective antigens to develop a multi-component vaccine. Conventional approaches to vaccine development are time consuming and identify only abundant antigens that may or may not provide immunity.

Reverse vaccinology (i.e. genomic-based approaches to vaccine development) can overcome these problems and enable the identification of novel vaccine candidates. All the OMPs targeted in the present study for vaccine development showed 98-100% homology in amino acid sequences among L. interrogans serovar Icterohaemmorhagiae, L. interrogans serovar Copenhageni and L. interrogans serovar Pomona. Interestingly, 13 putative OMPs did not show homology to other organisms in the NCBI database, except MCEI and II showed 25-30% similarities to MCE of M. tuberculosis (data not shown). The antibody response to MceII immunized animals showed lesser level compare to other recombinant proteins. Evaluation of immunogenicity of recombinant proteins with bovine sera from experimental and natural infection indicates that MCEII is highly immunogenic compare to other recombinant proteins of Leptospira (our unpublished data). Immunization of hamsters with denatured rMceII may cause lower level of antibody responses (FIG. 5). Although MCE did not provide immunoprotection in intracellular mycobacteria, we found the protective efficacy of rMCE II in a hamster model. MCE is involved in invasion of the Mycobacterium (Chitale et al. 2001. Recombinant Mycobacterium tuberculosis protein associated with mammalian cell entry. Cell. Microbiol. 3:247-254).

We have evaluated the protective potential of recombinant proteins using a hamster intraperitoneal challenge model. Hamster is a preferred model of leptospirosis disease and also allows us to study the protective efficacy against heterologous challenge. The study described here, in which we screened to assess the immunoprotection potential against Leptospira in a hamster model, suggests that rLP1454, rLP1118 and MCEII are protective antigens based on lethality. We have previously shown protection conferred by recombinant LigA protein (Palaniappan et al. 2006. Immunoprotection of recombinant leptospiral immunoglobulin-like protein A against Leptospira interrogans serovar Pomona infection. Infect. Immun. 74:1745-1750). To improve the protective efficacy of the recombinant antigens identified in this study, synergistic application of rLP1454, rLP1118 and rMCEII was attempted with rLigA and rGST as positive and negative control, respectively. The results imply a synergistic effect with better protection was obtained with all three antigens than alone.

In general terms, the predominant immunological effector response for an extracellular bacterium such as Leptospira is antibody-mediated whereby the bacteria are either killed directly via complement activation or opsonized for phagocytosis. Other mechanisms besides complement-mediated bactericidal activity may be responsible for the protection demonstrated in the present study and the contribution of other aspects of the immune system to the defense against leptospiral infection can also be assessed. A monovalent Leptospiral vaccine has been reported to induce type I protective immune response (Naiman, et al. 2002. Evaluation of type 1 immune response in naive and vaccinated animals following challenge with Leptospira borgpetersenii serovar Hardjo: involvement of WC1 (+) gammadelta and CD4 T cells. Infect. Immun. 70:6147-6157).

Recently, passive immunization of polyclonal antibodies of rLigA failed to provide protection against infection (Palaniappan, et al. 2005. Evaluation of lig-based conventional and real time PCR for the detection of pathogenic leptospires. Mol. Cell. Probes 19:111-117). It therefore appears that both humoral and cell mediated immunity can play a role in immunoprotection. The generation of the protective antigens identified in this study has the potential to enhance the efficacy of a recombinant based vaccine and can also lead to the development of multi-component vaccine to generate cross-protection against a wide range of increasing serovars of Leptospira.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes.

The citation of any publication is for its disclosure prior to the filing date and should not be construed as an admission that the present invention is not entitled to antedate such publication by virtue of prior invention.

What is claimed is:

1. A vector capable of expressing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above,
and wherein the recombinant DNA is inserted into the vector such that a recombinant protein is expressed when the vector is provided in an appropriate host.

2. A recombinant DNA vaccine comprising:
a) a recombinant DNA wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above; and
b) a vector capable of expressing the recombinant DNA when the recombinant DNA is inserted into the vector, wherein the recombinant DNA is inserted into the vector such that a recombinant protein is expressed when the vector is provided in an appropriate host.

3. A method for producing a vaccine against a Leptospira-related disorder comprising:
a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
b) providing a vector capable of expressing the recombinant DNA when the recombinant DNA is inserted into the vector; and
c) inserting the recombinant DNA into the vector, wherein the recombinant DNA is inserted into the vector such that a recombinant protein is expressed when the vector is provided in an appropriate host.

4. A method for producing a vaccine against a Leptospira-related disorder comprising:
a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (Lp328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
b) constructing an expression vector comprising a plasmid, wherein the plasmid comprises the recombinant DNA; and
c) inoculating a host with the expression vector, wherein the recombinant DNA is expressed in the host to produce a recombinant protein, and wherein the expressed protein raises an immune response in the host.

5. A vaccine comprising a recombinant protein, wherein the recombinant protein is selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 90% or 95% of the length of the amino acid sequence of any of the above.

6. A method for producing a vaccine against a Leptospira-related disorder comprising:
a) providing a recombinant DNA, wherein the recombinant DNA is selected from the group consisting of:
a recombinant DNA that encodes a protein having the amino acid sequence as shown in SEQ ID NO: 2 (LP1454),
a recombinant DNA that encodes a protein having the amino acid sequence as shown in SEQ ID NO: 4 (LP1118),
a recombinant DNA that encodes a protein having the amino acid sequence as shown in SEQ ID NO: 6 (LP1939),
a recombinant DNA that encodes a protein having the amino acid sequence as shown in SEQ ID NO: 8 (MCEII),
a recombinant DNA that encodes a protein having the amino acid sequence as shown in SEQ ID NO: 53 (CADF-like1),
a recombinant DNA that encodes a protein having the amino acid sequence as shown in SEQ ID NO: 55 (CADF-like2),
a recombinant DNA that encodes a protein having the amino acid sequence as shown in SEQ ID NO: 57 (CADF-like3),
a recombinant DNA that encodes a protein having the amino acid sequence as shown in SEQ ID NO: 59 (Lp0022),
a recombinant DNA that encodes a protein having the amino acid sequence as shown in SEQ ID NO: 61 (Lp1499),
a recombinant DNA that encodes a protein having the amino acid sequence as shown in SEQ ID NO: 63 (Lp4337),
a recombinant DNA that encodes a protein having the amino acid sequence as shown in SEQ ID NO: 65 (LP328),
a recombinant DNA that encodes a protein having an amino acid sequence as shown in SEQ ID NO: 67 (L21),
a recombinant DNA that encodes an immunogenic epitope or immunologically active fragment of any of the above, and
a recombinant DNA that encodes a protein fragment of at least 10%, 20%, 30%, 40%, 50%, 60% 70%, 80%, 90% or 95% of the length of the amino acid sequence of any of the above;
b) providing a vector capable of expressing the recombinant DNA when the recombinant DNA is inserted into the vector;
c) inserting the recombinant DNA into the vector;
d) providing a bacterial strain;
e) transforming the vector into the bacterial strain such that a recombinant protein is expressed when the vector is transformed into the bacterial strain; and
f) harvesting the recombinant protein from the bacterial strain.

7. A T-cell epitope vaccine comprising a recombinant protein, wherein the recombinant protein comprises a T-cell epitope, and wherein the T-cell epitope comprises an amino acid peptide fragment of a protein selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 90% or 95% of the length of the amino acid sequence of any of the above.

8. The vaccine of claim 6 wherein the amino acid peptide fragment comprises nine to twenty amino acids.

9. A method for producing a T-cell epitope vaccine comprising:
a) providing a recombinant protein that comprises a T-cell epitope, wherein the T-cell epitope comprises an amino acid peptide fragment of a protein selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and a protein fragment of at least 10%, 20%, 30%, 40%, 50%, 60% 70%, 80%, 90% or 95% of the length of the amino acid sequence of any of the above;
b) identifying the T-cell epitope from the protein;
c) inserting DNA encoding the T-cell epitope into a construct capable of expressing the T-cell epitope as a protein; and
d) harvesting the protein.

10. The method of claim 9 wherein the amino acid peptide fragment comprises nine to twenty amino acids.

11. A method for preventing a Leptospira-related disorder in an animal or human subject in need thereof, comprising administering to the subject an amount of the vaccine of claim 2, 5 or 7 wherein the amount is sufficient to confer immunity to the Leptospira-related disorder to the subject.

12. A pharmaceutical composition comprising:
a) a therapeutically effective amount of the vaccine of claim 2, 5 or 7; and
b) a pharmaceutically acceptable carrier.

13. An isolated and purified antibody directed against a Leptospira outer membrane protein, wherein the Leptospira outer membrane protein is selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 8% 90% or 95% of the length of the amino acid sequence of any of the above.

14. The antibody of claim 13 wherein the antibody is directed against at least one T-cell epitope of the Leptospira outer membrane protein.

15. A method for producing an antibody directed against a Leptospira outer membrane protein the method comprising:
a) providing a host animal; and
b) immunizing the host animal by injection with the Leptospira outer membrane protein, wherein the Leptospira outer membrane protein is selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 90% or 95% of the length of the amino acid sequence of any of the above,
and wherein the antibody specifically recognizes at least one T-cell epitope of the Leptospira outer membrane protein.

16. A method for diagnosing a Leptospira-related disorder in a subject comprising:
a) providing one or more antibodies directed against a Leptospira outer membrane protein, wherein the Leptospira outer membrane protein is selected from the group consisting of:
a protein having the amino acid sequence as shown in SEQ ID NO: 2 (LP1454),
a protein having the amino acid sequence as shown in SEQ ID NO: 4 (LP1118),
a protein having the amino acid sequence as shown in SEQ ID NO: 6 (LP1939),
a protein having the amino acid sequence as shown in SEQ ID NO: 8 (MCEII),
a protein having the amino acid sequence as shown in SEQ ID NO: 53 (CADF-like1),
a protein having the amino acid sequence as shown in SEQ ID NO: 55 (CADF-like2),
a protein having the amino acid sequence as shown in SEQ ID NO: 57 (CADF-like3),
a protein having the amino acid sequence as shown in SEQ ID NO: 59 (Lp0022),
a protein having the amino acid sequence as shown in SEQ ID NO: 61 (Lp1499),
a protein having the amino acid sequence as shown in SEQ ID NO: 63 (Lp4337),
a protein having the amino acid sequence as shown in SEQ ID NO: 65 (Lp328),
a protein having an amino acid sequence as shown in SEQ ID NO: 67 (L21),
an immunogenic epitope or immunologically active fragment of any of the above, and
a protein fragment of at least 10%, 20%, 30%, 40%, 50%, 60% 70%, 80%, 90% or 95% of the length of the amino acid sequence of any of the above;
b) collecting a cell or tissue sample from the subject;
c) contacting the sample with the antibodies; and
d) assaying for the presence or activity of the Leptospira outer membrane protein in the sample using the antibody to the Leptospira outer membrane protein,
wherein presence or activity of the Leptospira a outer membrane protein is indicative of the presence of the Leptospira-related disorder in the subject.


SEQUENCE LISTING (67 sequences)

SEQ ID NO: 1 - DNA, 1077 bp, Leptospira interrogans
  misc_feature (1)..(1077): serovar Pomona
  ref <308>: DATABASE ACCESSION NUMBER: GenBank / AAN48653
  ref <309>: DATABASE ENTRY DATE: 2006-02-01
  ref <313>: RELEVANT RESIDUES IN SEQ ID NO: (1)..(1077)
gtggggaaaaacaattcgttcagaccgaaaaattctttcttaaagttccaagttgtattcggtttttttcttttgagttttatttctttaaattcagaagaaagatcctcaattttagatcctacgttttttttaattcataagaatctgaatccttctttaggaggtttattcgaaaccagaaaaaaatttacttcttatggagcctggtttgaacttccagtttggaaacgagaacgtaaacatttttggaaaacccattttctttatgagaatcacaagactttttcagtcgttcatcaaacttacataggacttgaatattcttttatttcagaaaagaaggatctgcacccttccgtttttgtaggttgggaaagaggagaaaaggatttaggaattttcggaatccaccttacaattccggacaaacaaacgatccaagtttttggaaaaactggaaaagattttaaaagtggttctatctttcttcattctaactttgatactggacttcaattatttttaggattttcaaggacatgggacaaatcctataccgaagatcaattcactcttggaatcagggtttcttgggaaaaaatttactcttcttttttctggaatcaaacgcaggaagaagaatcatttctaaccgggaaatttggaatcagtaatttccaacaagatctcaaatctaaaacactttttgaatccacagacgaatttcagtcgaattcactttttaaatctaaaaatttaagttctaaaaacacaaaatcaaaatcgttcgaaacttctcttttagaaaaagataaaacaaattattcctacataagttcatttattcgtagttatacgaatatttcgatttccgttcaggaattattgtccgccggttttactctttcctccgctttggaaatttctaaggcgagctataactctaaagaagaatttctaaagttatttcattctctttcagtaaaagaacaaaccaaaattttcgttcttttgaaaaagaaaaatccgaagcacattcttaaaaattcaattcttcccaaagataaaagatga

SEQ ID NO: 2 - protein, 358 aa, Leptospira interrogans
MGKNNSFRPKNSFLKFQVVFGFFLLSFISLNSEERSSILDPTFFLIHKNLNPSLGGLFETRKKFTSYGAWFELPVWKRERKHFWKTHFLYENHKTFSVVHQTYIGLEYSFISEKKDLHPSVFVGWERGEKDLGIFGIHLTIPDKQTIQVFGKTGKDFKSGSIFLHSNFDTGLQLFLGFSRTWDKSYTEDQFTLGIRVSWEKIYSSFFWNQTQEEESFLTGKFGISNFQQDLKSKTLFESTDEFQSNSLFKSKNLSSKNTKSKSFETSLLEKDKTNYSYISSFIRSYTNISISVQELLSAGFTLSSALEISKASYNSKEEFLKLFHSLSVKEQTKIFVLLKKKNPKHILKNSILPKDKR

SEQ ID NO: 3 - DNA, 951 bp, Leptospira interrogans
  misc_feature (1)..(951): serovar Pomona
  ref <308>: DATABASE ACCESSION NUMBER: GenBank / AAN48316
  ref <309>: DATABASE ENTRY DATE: 2006-02-01
  ref <313>: RELEVANT RESIDUES IN SEQ ID NO: (1)..(951)
gtgaaacatccaaaaatgaacctcttgggagcgcttttacttagctccttattcttaaatcctttgaattccgaccctacgttaggcaactgtgaagtatttccaacaaataatatttggaatactcccgtagatacacttcccctgcatcctttttcagaatcttatgttcgaagtattggagcacaaaaaaaattaaaagcggatttcggctctgggctttgggagggaatgccaattggaattccatttatactaacgtctggtgccaatcctgtcccagtatcttttgaatatactgatgaaagtgaacctggaccgtatccaattccacataacgcacctatagaaggtggagaaacaagcgacggagacaggcacgttctagttgtagagcaaaaaacatgcaagttatatgaattgtattccgcaaggaaaaaaggaaaatcttggactgctgtgtctggtgcggtctttgatttgaagtccaatcaacttcgtccggctaactggacttctgcggacgcagccggactaccaattttacctggtttagtgagatacgaagaaatagcttctggagaaatcaaacacgcaattcgtttcaccgctaaaaaaacacaaaaagcgtatctatggcctgctcgtcattacgcttctaaaatcacagacaaaaacgtcccaccgatgggaactcgtttccgtttaaaagcaagttttaacatagacggatttagtaaagaaaaccaagtaattctacgtgcattaaaaaaatatggaatgatacttgccgacaacggatcagattggtttttatccggagctccaaacgaaaaatggaacaatgatcaacttcataaactaggtaaggttttaggagatcagttcgaagcagtagattcagaaagtttaatgatttcgacagattccggagaagccaaacaaaactaa

SEQ ID NO: 4 - protein, 316 aa, Leptospira interrogans
MKHPKMNLLGALLLSSLFLNPLNSDPTLGNCEVFPTNNIWNTPVDTLPLHPFSESYVRSIGAQKKLKADFGSGLWEGMPIGIPFILTSGANPVPVSFEYTDESEPGPYPIPHNAPIEGGETSDGDRHVLVVEQKTCKLYELYSARKKGKSWTAVSGAVFDLKSNQLRPANWTSADAAGLPILPGLVRYEEIASGEIKHAIRFTAKKTQKAYLWPARHYASKITDKNVPPMGTRFRLKASFNIDGFSKENQVILRALKKYGMILADNGSDWFLSGAPNEKWNNDQLHKLGKVLGDQFEAVDSESLMISTDSGEAKQN

SEQ ID NO: 5 - DNA, 480 bp, Leptospira interrogans
  misc_feature (1)..(480): serovar Pomona
  CDS (1)..(480)
  ref <308>: DATABASE ACCESSION NUMBER: GenBank /  AAN49138
  ref <309>: DATABASE ENTRY DATE: 2006-02-01
  ref <313>: RELEVANT RESIDUES IN SEQ ID NO: (1)..(480)
atgaaaaaacattctatcagtaaaatcattataactgcttgttgcatttttcttttaacatacggatgcaaacaagatccagtagattacaataataaaatcatggaaatcatgaacgcttctacaaacgatttagatgcgttaaacgcagccatggaaaaggaagaccttacaaacgcagaaaatgttagaaaagcttgggaaacaaagctagtttcttcactcgataagcttaaaggaatcagtgattttaaaggagattccagttttaaaaatgcaagcgtccaagctctcgaaacttatttgaacatagtaagtaaagactacaaacgtttgatcgaattacgaggattaggtgacaaagcagactcaaatgaaatcaaccaagttctcaatcgtattaatcaggattttgaaaaagctgcaaatactctcaatgctgcttctgataaatttgcgaaagaatacgcttctcaataa

SEQ ID NO: 6 - protein, 159 aa, Leptospira interrogans
MKKHSISKIIITACCIFLLTYGCKQDPVDYNNKIMEIMNASTNDLDALNAAMEKEDLTNAENVRKAWETKLVSSLDKLKGISDFKGDSSFKNASVQALETYLNIVSKDYKRLIELRGLGDKADSNEINQVLNRINQDFEKAANTLNAASDKFAKEYASQ

SEQ ID NO: 7 - DNA, 801 bp, Leptospira interrogans
  misc_feature (1)..(801): serovar Pomona
  CDS (1)..(801)
atgaatctttccaaacacacagccgttctcacagggattgttttcttcttagcattttccttaagtatgtatgtctccgttattgaaaaagccggaaccaaagacgaatatccatataccatgaaaatttattatccccgtttggaaggaattcatccaggtgcacccgttcgaattttaggagtagaaaaaggaattgtacgtagtttagacgtggttccgattgacgaagtagaagatcaaagattcctcaataaggatcaaacgaaggcaatcgaaattattgttagactgaaagagccaatcacactctgggacaattataaaattacattccaaaccaacacgattctctctggaagaaccatcgatatagaccctggatctttcgataaagaagagacgtccttttttcaacctacttacttagaagaagaacaaaaatctccagacttcttaccttctgcagattactttgaagattttttcgcggcttccacgggagtcatccgagaaaatcgtgaagacatccgaacttcttttaataatttttatgaaatttcagaaaaattgaaatcaaatcgaggaacaattcctcagatcattaactctccggaaacgtacgataacgtgatagaattacttacagatgctagaattttcggtaacgatgctcgtcgttatctggaagggaaccgtaagttggaacgttctgctccgattccactcacgattaatatgtatcgtagaactactttgatcggaaacgtgagtaatcggtattatttcggaaagttataa

SEQ ID NO: 8 - protein, 266 aa, Leptospira interrogans
MNLSKHTAVLTGIVFFLAFSLSMYVSVIEKAGTKDEYPYTMKIYYPRLEGIHPGAPVRILGVEKGIVRSLDVVPIDEVEDQRFLNKDQTKAIEIIVRLKEPITLWDNYKITFQTNTILSGRTIDIDPGSFDKEETSFFQPTYLEEEQKSPDFLPSADYFEDFFAASTGVIRENREDIRTSFNNFYEISEKLKSNRGTIPQIINSPETYDNVIELLTDARIFGNDARRYLEGNRKLERSAPIPLTINMYRRTTLIGNVSNRYYFGKL

SEQ ID NO: 9 - DNA, 1263 bp, Leptospira interrogans
  misc_feature (1)..(1263): serovar Pomona
  CDS (1)..(1263)
atgctcaacacaaaaataaagaaaaaattatttctctcagttattttttttctatttctaccatcggtcgcctgtgcggaaaaaagttttcgtaaacatatcgcggactcaaaactcattccttccgaaatcgaattttattctaacattcttcccggactttccggaaaaaatgtcattctaattacaaacccatctggaatcggaagaagccccgaaaggattttacgagaatttaaaaaacacgacgtaaaaatcaaacatctcatcggattggaacacggatttttaggactcgaggaggacttcagtaaatctcccgttacggtggatgaattttttaatctcccaatctatcatatctatcgagtcaagaacgcagaacttcctacgattttgaaaggagccgacgcgattctttttgatgtgcaagatatggggatgagatgttatacttatctaaccgttttaaaaagaattatggatggaattccagatcctacaaatacgagactgatcgttttggatcacgtaaaccccgctctttatttaaaaggaagaggagaaatgatcgataaacgtttcttaaattttgcaggagaattcccttctcttttttttggaggtttgaccttgggagaatcagcggtttattataactctgaatatttagataaaaaggttcgtttagaagtggtatctcctaaaaacgcaaaacgatcttttgattgggatagagaaggaattccttggactacaccttctcctaatttaccaactatagattctgcgatcaattatctggggcttgttttgttagaaggagtgaacgtttctgtgggaaggggtacaaccgcaccttttgtatatttcggagcgccttggatgacagagcccgaaaagttagcggaagaattaaatcaaaattcaggtggagaatattattatcagactgtgttttttaaacctgtatttggtccttacaagaatgagatttgtagaggattgcgcctaacggtagtaaatcgaaagtatgatcctttaaaaatggcattccagttgatctccgctttaaaatcgaattataaagaatttaaatggagatcgtatccggatggaacctacaatatagatttcctatggggaacggaatcgtttcgaaaaacgatcgacgccggaaaaaaatacgatcaatatgcggaatacttaagttccattgagaaagaatataatgaaaaaattaagaaatattatctctactga

SEQ ID NO: 10 - protein, 420 aa, Leptospira interrogans
MLNTKIKKKLFLSVIFFLFLPSVACAEKSFRKHIADSKLIPSEIEFYSNILPGLSGKNVILITNPSGIGRSPERILREFKKHDVKIKHLIGLEHGFLGLEEDFSKSPVTVDEFFNLPIYHIYRVKNAELPTILKGADAILFDVQDMGMRCYTYLTVLKRIMDGIPDPTNTRLIVLDHVNPALYLKGRGEMIDKRFLNFAGEFPSLFFGGLTLGESAVYYNSEYLDKKVRLEVVSPKNAKRSFDWDREGIPWTTPSPNLPTIDSAINYLGLVLLEGVNVSVGRGTTAPFVYFGAPWMTEPEKLAEELNQNSGGEYYYQTVFFKPVFGPYKNEICRGLRLTVVNRKYDPLKMAFQLISALKSNYKEFKWRSYPDGTYNIDFLWGTESFRKTIDAGKKYDQYAEYLSSIEKEYNEKIKKYYLY

SEQ ID NO: 11 - protein, 266 aa, Leptospira interrogans; identical to SEQ ID NO: 8
  ref <308>: DATABASE ACCESSION NUMBER: GenBank / AAN47806
  ref <309>: DATABASE ENTRY DATE: 2006-02-01
  ref <313>: RELEVANT RESIDUES IN SEQ ID NO: (1)..(266)

SEQ ID NO: 12 - protein, 251 aa, Leptospira interrogans
  ref <308>: DATABASE ACCESSION NUMBER: GenBank / AAN49254
  ref <309>: DATABASE ENTRY DATE: 2006-02-01
  ref <313>: RELEVANT RESIDUES IN SEQ ID NO: (1)..(251)
MNMNSLRYLLVGIIFTAAITVVGYFTIITEGGPIKKKGEFMKVTFRNAEGIKVGNKVTVQGVPFGYVSAIRLIQIDENGTEVQSGEMGIGTRVEITMLLREKISLYDNYDIIIKNESLLTGRVIAIDPGTADLEPKQLKTRTTPITMIDYKTTGSLKGRVLQDPLVSLSELISENRGDIRKTFSNIADITTKINTGDGSLGRLINNDDVHKNVNTVLTDAQIVLRELREGLEDTREQTPVTSFIRAALSAF

SEQ ID NO: 13 - DNA, 1323 bp, Mycobacterium tuberculosis
  misc_feature (1)..(1323): CDC1551
  ref <308>: DATABASE ACCESSION NUMBER: GenBank / AE000516
  ref <309>: DATABASE ENTRY DATE: 2004-08-04
  ref <313>: RELEVANT RESIDUES IN SEQ ID NO: (91)..(879)
gtgcgacgtcgtgctcaaggtcaacggcaagggcggccagccggtgtacatcaagctggccggtcaggacagcgggcggtgcgcgccgaaatgaaatccttcgccgaacgcaaccgtctggccatcggcacagtcggcatcgtcgtcgtcgccgccgttgcgctggccgcgctgcaataccagcggctgccgtttttcaaccagggcaccagggtctccgcctatttcgccgacgccggcgggctgcgcaccggcaacaccgtcgaggtctccggctatccggtgggaaaggtgtccagcatctcgctcgacggaccgggcgtgctggtggagttcaaggtcgacaccgacgtccgactcggaaaccgcaccgaagtggcaatcaaaaccaagggcttgttgggcagcaagttcctcgacgtcaccccccgcggggacggccgactcgattctccgatcccgatcgagcggaccacgtcgccctaccaactgcccgacgcccttggcgatttggccgccacgatcagcgggttgcacaccgagcggctgtccgaatcgctggccaccctggcgcagacctttgccgatacgccggcgcacttccgcaacgccatacacggggtggcccggctcgcccaaaccctcgatgagcgcgacaaccaactgcgcagcctgctggccaacgcggccaaagccaccggggtgctggccaaccgcaccgaccagatcgtcggcctggtgcgcgacacgaatgtggtcttggcgcagctgcgcacccaaagcgccgccctggaccggatctgggcgaacatctcggcggtggccgaacaactgcggggcttcatcgctgagaaccgccagcagctgcgcccggcgctggacaagctcaacggggtgctggctatcgtcgaaaaccgcaaagagcgtgtgcggcaggccatcccgctgatcaacacctatgtcatgtcgctgggtgagtcgctgtcgtcgggcccgttcttcaaggcatacgtggtgaacctgctgccgggtcagttcgtgcaaccgttcatcagcgccgcgttctccgacctggggctcgacccggccacgttgctgccgtcgcagctgaccgacccaccgaccggtcaacccggaaccccgccgttgccgatgccctacccgcgcacgggccagggcggtgagccgcggctgacgctgcccgacgcgatcaccggcaatcccggcgatccgcgctatccgtaccggccggagccgcccgcgccgccgcccggcgggccgccgcccggcccgcccgcgcagcagccgggagaccaaccgtga

SEQ ID NO: 14 - protein, 440 aa, Mycobacterium tuberculosis
MRRRAQGQRQGRPAGVHQAGRSGQRAVRAEMKSFAERNRLAIGTVGIVVVAAVALAALQYQRLPFFNQGTRVSAYFADAGGLRTGNTVEVSGYPVGKVSSISLDGPGVLVEFKVDTDVRLGNRTEVAIKTKGLLGSKFLDVTPRGDGRLDSPIPIERTTSPYQLPDALGDLAATISGLHTERLSESLATLAQTFADTPAHFRNAIHGVARLAQTLDERDNQLRSLLANAAKATGVLANRTDQIVGLVRDTNVVLAQLRTQSAALDRIWANISAVAEQLRGFIAENRQQLRPALDKLNGVLAIVENRKERVRQAIPLINTYVMSLGESLSSGPFFKAYVVNLLPGQFVQPFISAAFSDLGLDPATLLPSQLTDPPTGQPGTPPLPMPYPRTGQGGEPRLTLPDAITGNPGDPRYPYRPEPPAPPPGGPPPGPPAQQPGDQP

SEQ ID NO: 15 - protein, 9 aa, Artificial Sequence
  Synthesized sequence
VQGEESNDK

SEQ ID NO: 16 - DNA, 22 bp, Artificial Sequence
  Synthesized sequence
cgcggatccacgttaggcaact

SEQ ID NO: 17 - DNA, 21 bp, Artificial Sequence
  Synthesized sequence
ccgctcgagttagttttgttt

SEQ ID NO: 18 - DNA, 24 bp, Artificial Sequence
  Synthesized sequence
cgcggatccaatccgataacgaat

SEQ ID NO: 19 - DNA, 26 bp, Artificial Sequence
  Synthesized sequence
ccgctcgagttaatgatattcgtttt

SEQ ID NO: 20 - DNA, 24 bp, Artificial Sequence
  Synthesized sequence
cgcggatccagagttccttttttt

SEQ ID NO: 21 - DNA, 22 bp, Artificial Sequence
  Synthesized sequence
ccgctcgagttaaggaacgttt

SEQ ID NO: 22 - DNA, 21 bp, Artificial Sequence
  Synthesized sequence
tttggatccgcgattaccgta

SEQ ID NO: 23 - DNA, 21 bp, Artificial Sequence
  Synthesized sequence
cttctcgagttaaaagcactt

SEQ ID NO: 24 - DNA, 23 bp, Artificial Sequence
  Synthesized sequence
cgcggatcccccgacttaaatgt

SEQ ID NO: 25 - DNA, 23 bp, Artificial Sequence
  Synthesized sequence
ccgctcgagtcaaagtatcgaat

SEQ ID NO: 26 - DNA, 26 bp, Artificial Sequence
  Synthesized sequence
cgcggatccgtcaataacaacattgc

SEQ ID NO: 27 - DNA, 27 bp, Artificial Sequence
  Synthesized sequence
ccgctcgagttggtttccttttacgtt

SEQ ID NO: 28 - DNA, 21 bp, Artificial Sequence
  Synthesized sequence
cgcggatccatgttgttacac

SEQ ID NO: 29 - DNA, 25 bp, Artificial Sequence
  Synthesized sequence
ccgctcgagctactgagaaatcttt

SEQ ID NO: 30 - DNA, 21 bp, Artificial Sequence
  Synthesized sequence
cgcggatcctctcccgaatgg

SEQ ID NO: 31 - DNA, 24 bp, Artificial Sequence
  Synthesized sequence
ccgctcgagttatctcgtatcaaa

SEQ ID NO: 32 - DNA, 24 bp, Artificial Sequence
  Synthesized sequence
cgcggatccatagtacgtttaaaa

SEQ ID NO: 33 - DNA, 24 bp, Artificial Sequence
  Synthesized sequence
ccgctcgagttaaaaactgtggga

SEQ ID NO: 34 - DNA, 23 bp, Artificial Sequence
  Synthesized sequence
cgcggatcccaatcaaaatcggc

SEQ ID NO: 35 - DNA, 25 bp, Artificial Sequence
  Synthesized sequence
ccgctcgagttaatcctctaaatct

SEQ ID NO: 36 - DNA, 21 bp, Artificial Sequence
  Synthesized sequence
tcccccggggctggcaaaaga

SEQ ID NO: 37 - DNA, 23 bp, Artificial Sequence
  Synthesized sequence
ccctcgagaatatccgtattaga

SEQ ID NO: 38 - DNA, 27 bp, Artificial Sequence
  Synthesized sequence
aggggaattctatgaaaaactttcgat

SEQ ID NO: 39 - DNA, 22 bp, Artificial Sequence
  Synthesized sequence
ccctcgagcttagtcgcgtcag

SEQ ID NO: 40 - DNA, 23 bp, Artificial Sequence
  Synthesized sequence
cgcggatccgatcagatcaactt

SEQ ID NO: 41 - DNA, 25 bp, Artificial Sequence
  Synthesized sequence
ccgctcgagttaattttgtgttttt

SEQ ID NO: 42 - DNA, 21 bp, Artificial Sequence
  Synthesized sequence
cgcggatcctgcaaacaagat

SEQ ID NO: 43 - DNA, 25 bp, Artificial Sequence
  Synthesized sequence
cgcctcgagttattgagaagcgtat

SEQ ID NO: 44 - DNA, 22 bp, Artificial Sequence
  Synthesized sequence
cgcggatccgaaaaagccggaa

SEQ ID NO: 45 - DNA, 26 bp, Artificial Sequence
  Synthesized sequence
ccgctcgagttataactttccgaaat

SEQ ID NO: 46 - DNA, 24 bp, Artificial Sequence
  Synthesized sequence
cgcggatcctttccagaaatttct

SEQ ID NO: 47 - DNA, 25 bp, Artificial Sequence
  Synthesized sequence
ccgctcgagttaataatatctttgt

SEQ ID NO: 48 - DNA, 21 bp, Artificial Sequence
  Synthesized sequence
cgcggatccattgcacaaatc

SEQ ID NO: 49 - DNA, 24 bp, Artificial Sequence
  Synthesized sequence
ccgctcgagtcaaagagcaaactt

SEQ ID NO: 50 - DNA, 22 bp, Artificial Sequence
  Synthesized sequence
cgcggatccgaagaaagatcct

SEQ ID NO: 51 - DNA, 24 bp, Artificial Sequence
  Synthesized sequence
ccgctcgagtcatcttttatcttt

SEQ ID NO: 52 - DNA, 2064 bp, Leptospira interrogans
  misc_feature (1)..(2064): serovar Pomona
  CDS (1)..(2064)
atgcgtcctttttctaaattaatttttattctggccttttgtatttttttgcccgttttctctcaacctctaccggaccttccggaaaaacaatttggtcaacctcttaatacacaaaacgacgaatacaatcctatagtaagcccagatggaagatacatcgtattccagtccaaccgtccaggcggagaaggaggaatggacatttggatttccgagaacattcgctttttagataaggagataccagcagaatggactaaacccgtaaatatgaatcaaaatatttgggaagaattaaaacgtccaccagctgctggagttcgtaaaccaaatctattcaactctaacgcgtttgaaggtggaatttcaattcttttcgattctaacaacgcgccttcggaaatttattttacttctacaattaaccttgctgttgggcgttccggttttgaaggtttgaatatttatagaacaattaaagataaaaaaacaggaagatggacagacccagaacatctcagtgaaattaattccaacttcaatgataagatgcccgcaatttctcccgatggaaattttttgatcttctcttcggaccgtccggggggttacggtgatttcgatctttggatctcggttcgtaatccaaaaaacggaagttggtcccaacctaaaaatttaggttctcccctcaactcttccgaaagtgaaattcttcctttcattcatcaagacggagaacaactttatttcagttctaatcgagaagacgaaagaaaaaaatttaagatttttagaatattcttaaaatataaatctgctctagacaacatgttagaagacgaagaagaaaccgaagaaactcctacaaccaaaccgactgaaattttaattcctaaaattgatcaatcttctttattacttcttcccaaaccgtttaacactgataagtgggaaggttttgataacgaaggaatcagttttgacaaagacggtatctgggcttatatttcttccaatcgttctggtggagaaggtcaatttgacattttccgctttcaagttccggaatctattcgcaactcctatactttaaacttcaaaggtctagttttggatggttccgaaaagacgatgattggattagattctactttaaaaatttatgatggaactaaaccggctaacgtaatcacttcgaaaagaatcggaggagatcttaccaaaggaaaaccttctaattttgcaacaactcttcaaaccggaaaggtttataaaatagaaattagttctcctggctttcatcctcaagaggatattttggatttaagaggaaacataggtaaaaatcgaaaagtttatagaacctacgttcttttaccaattcaagttgaagaaggtaaaacggaagaaacaaaaatagaacaacccatagagaatcaaaaaccaaattctgccgcgttgaaagtcatcgtagcagacgcatctacaaaacaaatcataccagacgctaaagttactctttttacacctatgaaccgcaaaggagaatctcttgtccaagatgcggataaaaaatcttttcttattaaaaaattaccagataacgattttgaattatttgcaacagcttcgaaatatatttctgaaagtatcaatattattcaaaaaaatatttccaaaaatggaactgtaacaatttatctgaaagcagaaagcgacgtagatccggtttataatctacgagtttatttcgaatttaataaaacaaaaatcacagaagagaataaaaagttgctagatcctcttgtaggctatcttttgaaaaatgcgtccgataaaattgaaattgggggacataccgacaacgtagcctctaaggaatataacacaagattgagtgccaaaagagcccgtaacgtttacgagtatcttctttcaaaaggaattccggagaaaagaatgagaatcagggcttattggtattcacaaccggatgcagacaatgagacagaaaccggaagagcaaaaaatagaagagttggttttagaaagctctga

SEQ ID NO: 53 - protein, 687 aa, Leptospira interrogans
MRPFSKLIFILAFCIFLPVFSQPLPDLPEKQFGQPLNTQNDEYNPIVSPDGRYIVFQSNRPGGEGGMDIWISENIRFLDKEIPAEWTKPVNMNQNIWEELKRPPAAGVRKPNLFNSNAFEGGISILFDSNNAPSEIYFTSTINLAVGRSGFEGLNIYRTIKDKKTGRWTDPEHLSEINSNFNDKMPAISPDGNFLIFSSDRPGGYGDFDLWISVRNPKNGSWSQPKNLGSPLNSSESEILPFIHQDGEQLYFSSNREDERKKFKIFRIFLKYKSALDNMLEDEEETEETPTTKPTEILIPKIDQSSLLLLPKPFNTDKWEGFDNEGISFDKDGIWAYISSNRSGGEGQFDIFRFQVPESIRNSYTLNFKGLVLDGSEKTMIGLDSTLKIYDGTKPANVITSKRIGGDLTKGKPSNFATTLQTGKVYKIEISSPGFHPQEDILDLRGNIGKNRKVYRTYVLLPIQVEEGKTEETKIEQPIENQKPNSAALKVIVADASTKQIIPDAKVTLFTPMNRKGESLVQDADKKSFLIKKLPDNDFELFATASKYISESINIIQKNISKNGTVTIYLKAESDVDPVYNLRVYFEFNKTKITEENKKLLDPLVGYLLKNASDKIEIGGHTDNVASKEYNTRLSAKRARNVYEYLLSKGIPEKRMRIRAYWYSQPDADNETETGRAKNRRVGFRKL

SEQ ID NO: 54 - DNA, 1983 bp, Leptospira interrogans
  misc_feature (1)..(1983): serovar Pomona
  CDS (1)..(1983)
atgcataccctgctgactctgatcttaagccttttgcttttctctggtcttcagtctgagaataaaaattcttcctcaaaaaaactaagcgactctgcttcttggattcctaaagaaaattttactcaactcgcatcacaaagagaaaaattcaaaaacgtttctcataacgaaacattaaaactagaaattggctataaattattaactggaaaaattcttcagtttggaaaattacttagtaccgaaaacgcatacgtccccatcgaatccaacgaaattacttccgaactttctcaactcaacgacaagaccgtaagaattttatgcagtatgaaaggttccacctgcaatcctatacgttatgaaatttatcccttttggtattcgaaagaaatcaaaccttggacgatcaaaaaaattcctgattacgtaaatcataatatttttgcattcaatcctacagtttccccagacggtaaatatcttttttggaccgcttatgtcaaacgaggtaaatccggaactcaaaagatttggtattccaaattagacgaaaaaggtttttgggaagatggtaaggaaatgaacgctcctttaaataatgaaatgccttccgctgttatctctgcacttcctggtggtaacgaacttttcgtctttggaacgtttggcgaaaaagaacttttagacgaactcagtaaagattttgaaacgaaggcagacttggccgctcgttcttctaaaaattcaaacgtatatagaaaaaagatagaagaacttagagcagaatatgacgaaaaaacgaaacaaatttcaagcagagttcctctttataaaagttttaaggaaaaagattcctggtctaaaccaagcatattaaactttcctaatttttataatctttatagaaagagaaacgattcaagccaagaaattttcggtggatctactctttcttcttcagggagaattttaatctattcttctcaacataaggattccaaaggaaaattggatctttatgttagcaaaatgttaaacgacggaacctttcctttaggtacaaacttaggagatgtgatcaacacaactcatgaagagatggctccttttttagcaagcgatgatagaaccctttatttttctagcgacggtcataaagggctctctatttatatgacaaaaagaatcggagaaggttgggatcaatggaccaaacctgtcgaggtttccgaaaatctaaaaggtgtaaattttttttcaattcccgcgaatagcgactgggcctatataagcaaagacggccgattgtttatggcttatcttcctaaagaaatgcgcccagagaaggtcgtgatcataaacggaaaagttttagatacggatggtaaccctctttctgctgacatacattatgaatctttaaagtctcacgaaaagatcggtagcgccaaaagcgatccttctaatggtaatttttcgatcattctccccttcggcgaaaactacggtttttacgcccagaaaaaaggttatcttccagtatcacaaaatttgaatctaagttctaaaaagaaattctctgaaaaagtggaagtaattttacaacttcctccgatccgagaaagaggttccattcaaatcaataatttattttttgagtccaagagttttcaaatcgctccggaatccgccccagaactggatcgtctcgcggaaatcgtaaaggaaaatccagatattgaaattcagattgaaggtcataccgacaacattggcaagaaaaaagacaatctaattctttccgaaaaaagagcggcagcggtcgcagaatatctttttcaaaaacattctatttctaagactagaatccaaaccaaaggttttggggacagtgttcctttgagtaaaaatgattccgaagaagcacgcaaaaaaaacagaagggtcaattttacgattttgaaaaaaagtaaataa

SEQ ID NO: 55 - protein, 660 aa, Leptospira interrogans
MHTLLTLILSLLLFSGLQSENKNSSSKKLSDSASWIPKENFTQLASQREKFKNVSHNETLKLEIGYKLLTGKILQFGKLLSTENAYVPIESNEITSELSQLNDKTVRILCSMKGSTCNPIRYEIYPFWYSKEIKPWTIKKIPDYVNHNIFAFNPTVSPDGKYLFWTAYVKRGKSGTQKIWYSKLDEKGFWEDGKEMNAPLNNEMPSAVISALPGGNELFVFGTFGEKELLDELSKDFETKADLAARSSKNSNVYRKKIEELRAEYDEKTKQISSRVPLYKSFKEKDSWSKPSILNFPNFYNLYRKRNDSSQEIFGGSTLSSSGRILIYSSQHKDSKGKLDLYVSKMLNDGTFPLGTNLGDVINTTHEEMAPFLASDDRTLYFSSDGHKGLSIYMTKRIGEGWDQWTKPVEVSENLKGVNFFSIPANSDWAYISKDGRLFMAYLPKEMRPEKVVIINGKVLDTDGNPLSADIHYESLKSHEKIGSAKSDPSNGNFSIILPFGENYGFYAQKKGYLPVSQNLNLSSKKKFSEKVEVILQLPPIRERGSIQINNLFFESKSFQIAPESAPELDRLAEIVKENPDIEIQIEGHTDNIGKKKDNLILSEKRAAAVAEYLFQKHSISKTRIQTKGFGDSVPLSKNDSEEARKKNRRVNFTILKKSK

SEQ ID NO: 56 - DNA, 1428 bp, Leptospira interrogans
  misc_feature (1)..(1428): serovar Pomona
ttggaaagttttttacactttccttatgttttaaaggaatcttacgaaactaaaactatggggaaattttccaatcaaccttcaaaatccaaaatcataaacagacgtttacgaagaattgtatcatacgtttttattatttctacattgatcggttttacttctacttttccagaagaatcggacaaagttctctttcgttggaaactaaaaccaggagagaatgtagaattaaacgaatatcatcgagtacaattagtaagtatgggaaagaaaattagaagagaagataaaaatcgaatcctattacaaaccctttcctgcgaaaataaagaatgtacgttagaaggattttttgatacttacactcgttttcccgaagttgatcctgcgtttcgtaaagataaaacgtttaagagcagatttcaaataacagatttaggtcaatataaagtgccacaggaatacagtatgcctaatctccgctctcttcctagtttttccgaaaaaccaatttccattggagaagaatggacacaacccgctacggaaagttttcaatttccaggaggaagggttatgatcacggtttttgcgaaatataagtatcacggtgtagacgaatgggaatatcaaaagttgtccggtaaaggagatcgtatcgaatacaattataatttatattatgattctcaaatgaacagaaccggggttccatttaagatctacggatttgcgcgaggaatggtattttttgatcgcgaacttggaattccacaatataaaagggttcagttgtcgtacacttttgtctatgaaaatggaatggcgcaagagatgtcgttcgacattcacggagtatataataaaaacgtaaaacttacggatcaagacaaggacaagtttgcggaagaaatccgtaaaattttaggaggagaacttccaactggaattgaaccagattccgattcaaaaaagaatttaaaaaaaccgaaaaaggaaaccatttcatggccggaagaagaggataatcaagccgcaccagaaagttcgccggtagaaattagaagaacagaagaaggaatcgctatttctttaaattcagtgttgtttgatcacaacagctcagaacttaaacaagaagcaaaattagaattaaaaagaatcgcatccgtattgaaaaaatatgcagatagagaaatcagaattagcgggcatactgacaattcaggcggggaagaatataatcgaaaactttccagagaaagagcactttccgttttgaaggaactcagagatgaacaaggtttggaagaaaaaagaatgtcttacgaaggatatgggaaatcaaaaccgattgcagataactctacgattcaaggtaggcaaaaaaatcgtagggtagatattactattgtaatggaataa

SEQ ID NO: 57 - protein, 475 aa, Leptospira interrogans
MESFLHFPYVLKESYETKTMGKFSNQPSKSKIINRRLRRIVSYVFIISTLIGFTSTFPEESDKVLFRWKLKPGENVELNEYHRVQLVSMGKKIRREDKNRILLQTLSCENKECTLEGFFDTYTRFPEVDPAFRKDKTFKSRFQITDLGQYKVPQEYSMPNLRSLPSFSEKPISIGEEWTQPATESFQFPGGRVMITVFAKYKYHGVDEWEYQKLSGKGDRIEYNYNLYYDSQMNRTGVPFKIYGFARGMVFFDRELGIPQYKRVQLSYTFVYENGMAQEMSFDIHGVYNKNVKLTDQDKDKFAEEIRKILGGELPTGIEPDSDSKKNLKKPKKETISWPEEEDNQAAPESSPVEIRRTEEGIAISLNSVLFDHNSSELKQEAKLELKRIASVLKKYADREIRISGHTDNSGGEEYNRKLSRERALSVLKELRDEQGLEEKRMSYEGYGKSKPIADNSTIQGRQKNRRVDITIVME

SEQ ID NO: 58 - DNA, 1683 bp, Leptospira interrogans
  misc_feature (1)..(1683): serovar Pomona
  CDS (1)..(1683)
atgaaacaatttttatttttactgtgtgtatttctactctttcaaaattgttctgtcaagcccacggaaaatccgtgcgatacgagtacttctcttttttataaatctttgttcttaaattttattttaacttccggcaaaacgagtatttgcggtatgcacacgtcggttcagttgcctccccctacgattctcaacataaaatcaaaaagtactttgaatacaggatttttaattggtgaaatggatgagtctgcttccggagttcaaatttctttagattcgggtccatttatggacgctcaaacttctggcaatcagtggaaatttcaacttcctgccgcaggtgtttctactacaattccttcaagtggaatttggagagactggagtttacatacaatttcggttcgatctacttctaaggaatcaaattctattccgattacaatcactgttcaaaaaggttcaaacaaggatattaacggcgacggttatccagatgcattgattggttctcaagctgcaaatcgagttagggcttatctttctcttgggaaagcaaagggtttagatttggttccggtcactttactaaatggggcaggtggatttggttattccgtcaagttaggcgacatcgatggagatggatacgccgatgcagttgtaggaagtgcaacaaatacattcgctatttatttaggatcaatcggtgggctttcgacaacggcgattaattatatccctattggtgttggtggtttattgaatgtggatgtaggagatataaacggagacggattttcagacgttttgattggtgtaccttatgacgtaggaaatattgggcgtgtttattcttatttttcaaatggagttatggggcaaggtgttacgtttggtcaacaattgaacaatcctggaaatgcaggtagtgctgcattttacggatatgccattacgttaggtgatattaatggagatggtaaatcggatgcgattataggagcggtcggttctggacaggtcggcgcttcttttatttatcttgcgcaagcagcaggggcttttgctgcttattctcaaacaattacgggatcggctgcaaatgaatggtatgcaaattcagcaattgctaccgatataaaccgggatggatttgcggatttatttgtgggggcttatcaagaatcgggcggcgttggaagagtacatttatatttatctaatttaggaattcttgtgaataccgccaacggtccaatttcaggattggttgggtcacaaactggaacttctgtggcaactggagatgtaaacggtgatggatttttagatcttcttaccggaggttattcttacacttctacgtttccaaatcaaggttatgcgattactcatttaactacgggtgatacaatgggtttaactgtgaaccctttaaatcttctaacggttccagtgaatttaggagatatgggaaattcaattgcttcggtagatattaacggtgacggtttaagtgacgttcttgtgggagctcctagttccgtaggtggaacaaatgttggaaacgtttacctttatatatcaaacggattggacggatatacttcggccccacaaattttcgtcgaaccagatgtaaacggaacgtttgggacttccgtggatttatga

SEQ ID NO: 59 - protein, 560 aa, Leptospira interrogans
MKQFLFLLCVFLLFQNCSVKPTENPCDTSTSLFYKSLFLNFILTSGKTSICGMHTSVQLPPPTILNIKSKSTLNTGFLIGEMDESASGVQISLDSGPFMDAQTSGNQWKFQLPAAGVSTTIPSSGIWRDWSLHTISVRSTSKESNSIPITITVQKGSNKDINGDGYPDALIGSQAANRVRAYLSLGKAKGLDLVPVTLLNGAGGFGYSVKLGDIDGDGYADAVVGSATNTFAIYLGSIGGLSTTAINYIPIGVGGLLNVDVGDINGDGFSDVLIGVPYDVGNIGRVYSYFSNGVMGQGVTFGQQLNNPGNAGSAAFYGYAITLGDINGDGKSDAIIGAVGSGQVGASFIYLAQAAGAFAAYSQTITGSAANEWYANSAIATDINRDGFADLFVGAYQESGGVGRVHLYLSNLGILVNTANGPISGLVGSQTGTSVATGDVNGDGFLDLLTGGYSYTSTFPNQGYAITHLTTGDTMGLTVNPLNLLTVPVNLGDMGNSIASVDINGDGLSDVLVGAPSSVGGTNVGNVYLYISNGLDGYTSAPQIFVEPDVNGTFGTSVDL

SEQ ID NO: 60 - DNA, 3492 bp, Leptospira interrogans
  misc_feature (1)..(3492): serovar Pomona
ttgaacacatccatccaagttggttttagtcaaaaattggattcttctagtatccaatctcaatcgatccaactaacacaaggaaatacgatcattccgggaaatttcacttctacagaaaaaactcttttattcaatccaacgtcttccttggctgcatcaactgtatattccgttagcatttccaaagacataaaatcaatggacggctcctctctttcagaagactatacttggagctttaccaccaacacgattgttgatttagtcgcgccagacgtatctttaagaacccctaccataggtgccaatttagttcctaataatacttccgttcaaattgcgtttactgaaacaatgaattgcacttccatcaacattgtaaattttacattaaaaaataatgttaccaatgtattagaacctagtaatgtagtttgtttaggatccgttgccacccttacccctaataatcctcttgcttttaatactgtttatcgtgtggatattctttctacagcaaaagatcttgctaataatcctcttgtaaacgcatataactggacttttacaaccggagttgcccccgatttaacggttccaaccgtctcctttgtaaatcccactcccaatgcacaaaacgttcctatcaacgaaacaatcagtattgcctttagtgaacctatcaattgtgctacgatcatcggaagtattgtcttagatgataatatacttattcctggaagcgtaaacgggaatccgggttgtacaggcacaaccgcatcctttactcctttaggcaatttgacaccaaatacgaattatacggttaccgtctcaaatgcgattacggatttgcaaaacaatccacttacaccttctacctggagttttacgacggctgctgcagtcgatcagactcaacccacggttacatttactgtaccctccgccaacgcaaacggcgtagggaccaatgtcaaccctatggtagtttttagcgaacctatgtcttgcgcttccgtgacctccgcatcctttcgattaaagaggcaggcaaccggggtttatcttataggaagtgtaaattgttttggaacttccgctacctggaccccagatcctgtaaatccgcttgcctttaatactacttacaccgtagaaatcgatcagggtgcattagatacttttaataatcctctcattccaataaattggaactttactacaggcccaggacccgatctaactcctcccagtgttgccgtcgttactcccgcaaacgctgcgataggcgttccgaccaacggaggtgtaagtatcgcttttagtgaagccatgaattgtggaagtattttaggaggaatcaccctggacgatgatcctacaactccgggtactgtgattccaatcaatatcaattgtaacggaaatactgtctcttttgccccgacaatccctccacttgcctttaatacaacctacacagtaacgattttaaatacggttacggacagtaataataatgcactaaacggaggaaattacgcttggtcttttacaacaggagtggcaccagacctagttcctcctcaagtttctcttgtaagccctctctcgggagcagtaggtgtcgcaacaaatgcaaacattacagttgcatttaatgaaaccatcaattgttccaccttgaattttacggttaataacggaatcaatggtactgtgaactgttcaggatcctctgctacttttattcctagcgctttaacccctctaaatgccggtacaaattatacagcaacaatcctaacagtaaatgatattgtaggaaatccaatcggtgctgcgtttggttggagttttacaaccggagccgctccagatgtgactcctcctgtcgttacgatccaaaatcttagaaataattctatcgtagaaactggtttcgtaattggaacggcaacagatgcaggcacaattacttctgtagaagtttctttggacaatggcgcttttgtgcccgcaaccggaaccaatccttggaaatttaaacttccttcggatataaatacttggaagcaaaactctcaacatacgatcattgcaagagctaaggacttagccaataaccttacaactacagcagccatttcggttcgaaaaggaaacaataaagatataaacggagacggatacgttgatcttgtatctgcagaatacggacagggtttactttatatttttcattcttccgggaacgctggaatgacaattacaaacgcacaatccgcaagtaagatcattgtaggagtcgcggcggaagaatttggaagaactgtttctatgggagatttaaacggagatggatttgcagacgtaatttctggtgctcctggttggaacggtgcccaaggtagggtttatatatttcattcttccggaaacgctggagtcaatatttccttttccggttttgccactaaaacgatcagtggagccaatgcgggagctagatttggagatagtattgttacaggtgatttaaacggagacggctacgcagacctagcatcaggagaacccgtttttaatggttctcagggtagagtctatgtatttcattccgctggagccgctggagttacacaaataaattcggctgctgcaaattctacactcacaggtgaaaacgcaactgatcgtttcggatattcgttgagcactggaaatatgaacggagataattttgcagatcttgcaattggagcacctggttatggcgctggcgtaggtggaggatttgtagtaaaccaaggtaaagtttatatacatcacggggcggctggtggattgggaggagtaattacaactcttaccaatgatagcgctggaaatgctggggaatttggtatcagtttatttacagccgattttaacggagacggtaattccgatcttgcaattggaagtccaaatttagggggtggaactggaagagtttccgtatttacgtctgcaggaggtgtaggtataaatacatctacgattggaaacgctccgcttatgatcaatggaacggctgtgggtaatgcatttggaatttctttaaccgcacaggatttaaatttagatggaagaccggatttaatttccgcaaccgtaattcccaatagggtattcgttttccacatgccaggtgcaggagctatcggtggatttctaaccactggtaatgcaactacacaaatcacgagtgcatttgcaggaatcggggtttccccaaatgcacctaaaactcctatttccggcggagacatcaacggagacggatttccagacttatttgtcgggggaagttctgataatatttttattttccattcttcaacggttggaactggtttattaacaaatactactgcaaccgctgcaggcgcaattaccagttccggacttgccaatggcttttttggatgtagcgtttattga

SEQ ID NO: 61 - protein, 1163 aa, Leptospira interrogans
MNTSIQVGFSQKLDSSSIQSQSIQLTQGNTIIPGNFTSTEKTLLFNPTSSLAASTVYSVSISKDIKSMDGSSLSEDYTWSFTTNTIVDLVAPDVSLRTPTIGANLVPNNTSVQIAFTETMNCTSINIVNFTLKNNVTNVLEPSNVVCLGSVATLTPNNPLAFNTVYRVDILSTAKDLANNPLVNAYNWTFTTGVAPDLTVPTVSFVNPTPNAQNVPINETISIAFSEPINCATIIGSIVLDDNILIPGSVNGNPGCTGTTASFTPLGNLTPNTNYTVTVSNAITDLQNNPLTPSTWSFTTAAAVDQTQPTVTFTVPSANANGVGTNVNPMVVFSEPMSCASVTSASFRLKRQATGVYLIGSVNCFGTSATWTPDPVNPLAFNTTYTVEIDQGALDTFNNPLIPINWNFTTGPGPDLTPPSVAVVTPANAAIGVPTNGGVSIAFSEAMNCGSILGGITLDDDPTTPGTVIPININCNGNTVSFAPTIPPLAFNTTYTVTILNTVTDSNNNALNGGNYAWSFTTGVAPDLVPPQVSLVSPLSGAVGVATNANITVAFNETINCSTLNFTVNNGINGTVNCSGSSATFIPSALTPLNAGTNYTATILTVNDIVGNPIGAAFGWSFTTGAAPDVTPPVVTIQNLRNNSIVETGFVIGTATDAGTITSVEVSLDNGAFVPATGTNPWKFKLPSDINTWKQNSQHTIIARAKDLANNLTTTAAISVRKGNNKDINGDGYVDLVSAEYGQGLLYIFHSSGNAGMTITNAQSASKIIVGVAAEEFGRTVSMGDLNGDGFADVISGAPGWNGAQGRVYIFHSSGNAGVNISFSGFATKTISGANAGARFGDSIVTGDLNGDGYADLASGEPVFNGSQGRVYVFHSAGAAGVTQINSAAANSTLTGENATDRFGYSLSTGNMNGDNFADLAIGAPGYGAGVGGGFVVNQGKVYIHHGAAGGLGGVITTLTNDSAGNAGEFGISLFTADFNGDGNSDLAIGSPNLGGGTGRVSVFTSAGGVGINTSTIGNAPLMINGTAVGNAFGISLTAQDLNLDGRPDLISATVIPNRVFVFHMPGAGAIGGFLTTGNATTQITSAFAGIGVSPNAPKTPISGGDINGDGFPDLFVGGSSDNIFIFHSSTVGTGLLTNTTATAAGAITSSGLANGFFGCSVY

SEQ ID NO: 62 - DNA, 1287 bp, Leptospira interrogans
  misc_feature (1)..(1287): serovar Pomona
  CDS (1)..(1287)
atgagcctaaaaaataaaaattatgttttatcaaaaaaaaccatattgattctatttttagtatattttgtttttataatttcttttttttctatttactctcaggatttaagtataaatcaaaatcctaaaccagaaaagttaaaaggttctatcaatacgagtctcaatgagtttggaatcagtcttactgatgacggaaatattttatatttctattctaaaagacaaaattcaaattatacagacatttataagtcaactcgaacgaaagatgaatggacacaaggagaggaaattgaagttctaaattcaaactttgacgaccaaagtcctttcattttaaaccgagaagaaggaattcttttttcatctaatagagatggtgcgaccgaattccaatttgcaaatggaaaaatcggagtttctagagatatttatttttctaaaaaaataaattcttcttggacagaaccggttcttcttcctagagccgtgaatacggaagaaatcgaagaaaatccgtttctatttaataatagattgtattttacccgttatccttttgggcaagtttcagaagcggacattttcgtttctgtttataaaaataacacttgggaaaaagcaatgagcctccctgatccgattaacaccgtttattcggaaattgcggctacaattagtaaagatggaaagacgatttatttttcttctaaccgtccggggggttttggcggttatgatttgtataagtctactttacttgaaaacggaaattattccgaaccgattaatcttggacccgaaattaacactaccggagatgaggctttttttctggaaacaaacgatagaaagacattctatttttgtagaaggaaagaacgcgattatgatatttattctatcgtttctaacccgtttcaagaactagaaaaaggaaaatctatttctttggatagtatccatttttctttgggctcttatgaaattctcgaaaattctttttcaattttagataatttgaattcttatcttaaggaaaatttaaatataaaaatcaaaatcaccggccataccgatcttaatggagattcccaggacaaccttattctcagccgtaatcgtgcaaatgcagtaaaggattatttagttaaaaggggaatcgattctcaaagaattatcacggatgggaaaggtagttcggagccaattgttcctatgaaaaatccagagacggattataaaaatagaagaaccgaatttcagattatcagtcgttag

SEQ ID NO: 63 - protein, 428 aa, Leptospira interrogans
MSLKNKNYVLSKKTILILFLVYFVFIISFFSIYSQDLSINQNPKPEKLKGSINTSLNEFGISLTDDGNILYFYSKRQNSNYTDIYKSTRTKDEWTQGEEIEVLNSNFDDQSPFILNREEGILFSSNRDGATEFQFANGKIGVSRDIYFSKKINSSWTEPVLLPRAVNTEEIEENPFLFNNRLYFTRYPFGQVSEADIFVSVYKNNTWEKAMSLPDPINTVYSEIAATISKDGKTIYFSSNRPGGFGGYDLYKSTLLENGNYSEPINLGPEINTTGDEAFFLETNDRKTFYFCRRKERDYDIYSIVSNPFQELEKGKSISLDSIHFSLGSYEILENSFSILDNLNSYLKENLNIKIKITGHTDLNGDSQDNLILSRNRANAVKDYLVKRGIDSQRIITDGKGSSEPIVPMKNPETDYKNRRTEFQIISR

SEQ ID NO: 64 - DNA, 1152 bp, Leptospira interrogans
  misc_feature (1)..(1152): serovar Pomona
  CDS (1)..(1152)
atggcgaagaaagaaaactactatattactatcaaaggtagaaaatatgatcgtaagttgatccagctcgcggaagagttcacttccggtaaacgggacggtaagatttcgatcaacgacgcaaaacgtcttttaaaaattgtcaaggataacaacgcttatacggatatagaaaaacatacgatcgaatacattcgtgaaaactataagtttaccgaaaaatcggacgaatggttccgttcagaaatccgtaaatgggccgctaaaaaagtgcaagaagcaaaaaagaaaagtgatgtggaatccatcctagtcgatgattctgaagcgccggaaataaattttccttccagttggggagaagacaaaaccgaagttgttgaaattacacaaacctctaaaatagattggagagaaaattcaaatttttcatccgcaacgtctcactctaaaaaaaataaaaagatcattccaactttaatttttctttctggttttttgattcttgtaggtttagtttatttttttcgaaccttattttataaggaagacttggaacaagtagtaaaaacgaattctgagattgtctctaattcaaaagaaaaacaatccgacgtttcgatagaaaaagcagaatctacaaaggaagttcgaaagaaaaacgtaagatctaaaaaagaagaatcggaaattccaaaaaatgctcttacaattctaaaacctcaaaccggaaagaagttagaatctaaatccttattctcttcgttgacaaatcaaaattccacagaagaattttcttccaatcctcaatttagagaaattgaatccaatgtaattcgttttgaaaaaaacagcattcaaattcataaagaatcgagaccaagtctcaaccgtttggctcgctggatgaaacaggattcttcgatccgagtcaaagttatcggtcatacttctttagagggtagcgaagacgccaatcaaaaagtttctcttcttcgtgcacaaacggttcgaaattatatcgccgggaatggcatttccaaagatcgttttgagattattcccaaaggcgcaagcgttcctattggcgataattctaaagaagaggggaaggaaatgaatcgtagagtggaacttagaatctataattga

SEQ ID NO: 65 - protein, 383 aa, Leptospira interrogans
MAKKENYYITIKGRKYDRKLIQLAEEFTSGKRDGKISINDAKRLLKIVKDNNAYTDIEKHTIEYIRENYKFTEKSDEWFRSEIRKWAAKKVQEAKKKSDVESILVDDSEAPEINFPSSWGEDKTEVVEITQTSKIDWRENSNFSSATSHSKKNKKIIPTLIFLSGFLILVGLVYFFRTLFYKEDLEQVVKTNSEIVSNSKEKQSDVSIEKAESTKEVRKKNVRSKKEESEIPKNALTILKPQTGKKLESKSLFSSLTNQNSTEEFSSNPQFREIESNVIRFEKNSIQIHKESRPSLNRLARWMKQDSSIRVKVIGHTSLEGSEDANQKVSLLRAQTVRNYIAGNGISKDRFEIIPKGASVPIGDNSKEEGKEMNRRVELRIYN

SEQ ID NO: 66 - DNA, 549 bp, Leptospira interrogans
  misc_feature (1)..(549): serovar Pomona
  CDS (1)..(549)
atgaaatataaaataattttaattttatcactaatgttatttcttttcgtttcctgtccggatgaaaaaaaagaaaatgaattgagtacttatattttatatagtgttctcataaacgcaactactcaatacgattgtgttactagttcggaagtagtatcagactcttataacaaaacaacaataaccttcgaaaataaacctcaatattacaattcacccagtggaaatgtagttccaaaagcaattatgccgattttgattaaaaaggggcagacaattcaagtatccagtataacgactaacgttaagtatgaagcgacaaaccaagacttaacttttctttttagaaaagatggttgtcacggtacaaactccgaaattgcaacctatgcaggagctactaatacaaatgtttttttaggaaacacaaatactgttagcttaactcaatttaaatttaccgccgactataatgggattatactaatcgttgggaaaaacctaggtgcaagtttacctggagatattcgtgtgaatgtattttaa

SEQ ID NO: 67 - protein, 182 aa, Leptospira interrogans
MKYKIILILSLMLFLFVSCPDEKKENELSTYILYSVLINATTQYDCVTSSEVVSDSYNKTTITFENKPQYYNSPSGNVVPKAIMPILIKKGQTIQVSSITTNVKYEATNQDLTFLFRKDGCHGTNSEIATYAGATNTNVFLGNTNTVSLTQFKFTADYNGIILIVGKNLGASLPGDIRVNVF